(19) United States
(12) Patent Application Publication
(10) Pub. No.: US 2019/0367564 A1
(43) Pub. Date: Dec. 5, 2019

(54) COMPOSITIONS AND METHODS FOR USE OF ANION CHANNEL RHODOPSINS

(71) Applicants: THE BOARD OF REGENTS OF THE UNIVERSITY OF TEXAS SYSTEM, Austin, TX (US); UNIVERSITÄT ZU KÖLN (UNIVERSITY OF COLOGNE), Köln (DE)

(72) Inventors: John Lee SPUDICH, Houston, TX (US); Elena G. GOVORUNOVA, Houston, TX (US); Oleg A. SINESHCHEKOV, Houston, TX (US); Michael MELKONIAN, Köln (DE)

(21) Appl. No.: 16/461,788

(22) PCT Filed: Nov. 17, 2017
(86) PCT No.: PCT/US2017/062254

§ 371 Date: May 16, 2019

Related U.S. Application Data
(60) Provisional application No. 62/423,485, filed on Nov. 17, 2016.
(60) Provisional application No. 62/423,503, filed on Nov. 17, 2016.

(51) Int. Cl. C07K 14/405; C12N 15/86

(52) U.S. Cl. CPC C07K 14/405; A61K 38/00; C12N 2750/14143; C12N 15/86

(57) ABSTRACT

Methods and compositions used to identify and characterize novel rhodopsin domains, which are anion-conducting channelrhodopsins. The rhodopsin domain of these anion-conducting channelrhodopsins have been cloned, optimized and expressed in mammalian systems and thus may be used in, among others, optogenetic applications and as therapeutic agents for electrically active cell mediated disorders.

This application claims the benefit of U.S. Provisional Patent Application No. 62/423,485, filed Nov. 17, 2016, and U.S. Provisional Patent Application No. 62/423,503, filed Nov. 17, 2016, the entirety of which are incorporated herein by reference.

This invention was made with U.S. Government support under Grant Nos. U01MH109146 and R01GM027750, awarded by the National Institutes of Health. The government has certain rights in the invention.

### BACKGROUND OF THE INVENTION

### 1. Field of the Invention

This disclosure generally relates to the fields of molecular biology and medicine. Methods and compositions that utilize channelrhodopsins, such anion-conducting channelrhodopsins, are provided. In some aspects, channelrhodopsins can be used for optogenetic applications or as therapeutic agents.

### 2. Description of Related Art

Optogenetics (Deisseroth. Nat Methods 8 (1): 26-9, 2011, Deisseroth, K. Optogenetics: 10 years of microbial opsins in neuroscience. Nat. Neurosci. 18, 1213-1225, 2015), refers to using optical methods for probing and controlling genetically targeted neurons within intact neural circuits. Optogenetics involves the introduction of light-activated channels and enzymes that allow manipulation of neural activity with millisecond precision while maintaining cell-type resolution through the use of specific targeting mechanisms. Because the brain is a high-speed system, millisecond-scale temporal precision is central to the concept of optogenetics, which allows probing the causal role of specific action potential patterns in defined cells.

Light control of motility behavior (phototaxis and photophobic responses) in green flagellate algae is mediated by sensory rhodopsins homologous to phototaxis receptors and light-driven ion transporters in prokaryotic organisms. In the phototaxis process, excitation of the algal sensory rhodopsins leads to generation of transmembrane photoreceptor currents. When expressed in animal cells, the algal phototaxis receptors function as light-gated cation channels, which has earned them the name “channelrhodopsins”. Channelrhodopsins have become useful molecular tools for light control of cellular activity.

Originally, the source of these light-activated channels and enzymes were several microbial opsins, including Channelrhodopsin-2 (ChR2) a single-component light-activated cation channel from algae, which allowed millisecond-scale temporal control in mammals, required only one gene to be expressed in order to work, and responded to visible-spectrum light with a chromophore (retinal) that was already present and supplied to ChR2 by the mammalian brain tissue. The experimental utility of ChR2 was quickly proven in a variety of animal models ranging from behaving mammals to classical model organisms such as flies, worms, and zebrafish, and hundreds of groups have employed ChR2 and related microbial proteins to study neural circuits. Currently, several members of this family have been recruited as molecular tools for optogenetics, i.e. regulation of cellular activity with light. Phototaxis receptors from green (chlorophyte) flagellate algae (Sineshchekov et al., 2002), best known as channelrhodopsins (ChRs) owing to their function as light-gated cation channels are widely used to depolarize genetically targeted populations of excitable cells.

Hyperpolarizing rhodopsin ion pumps have been employed to suppress neuron firing, but they transport only a single charge per captured photon and therefore have limited capacity. Recently, ChRs were engineered to conduct Cl−, but these optogenetic tools still retain some cation conductance and could be made highly light-sensitive only at the expense of greatly slowing the channel kinetics with additional mutations (J. Wietek et al., Science 344, 409 (2014) and A. Berndt, S. Y. Lee, C. Ramakrishnan, K. Deisseroth, Science 344, 420 (2014)). Ideal for optogenetic hyperpolarization would be natural light-gated anion channels optimized by evolution to be highly conductive and anion-selective.

Described herein are modified and optimized rhodopsin domains derived from a newly identified class of channelrhodopsins, Anion Channel Rhodopsins (ACRs), which are light-gated anion channels that provide membrane hyperpolarization and neuronal silencing through light-gated chloride conduction, and depolarization in cells with high cytoplasmic chloride concentration.

### SUMMARY OF THE INVENTION

The presently disclosed methods and compositions are based, in part, on the discovery and identification of a novel class of channelrhodopsins, Anion Channel Rhodopsins (ACRs). Light-gated rhodopsin cation channels from chlorophyte algae have transformed neuroscience research through their use as membrane-depolarizing optogenetic tools for targeted photoactivation of neuron firing. Photosuppression of neuronal action potentials has been limited by the lack of equally efficient tools for membrane hyperpolarization. Described herein are modified and optimized rhodopsin domains derived from a newly identified class of channelrhodopsins, Anion Channel Rhodopsins (ACRs), light-gated anion channels that provide efficient membrane hyperpolarization and neuronal silencing through light-gated chloride conduction or in the case of neurons with high cytoplasmic chloride concentration, wherein the expressed rhodopsin depolarizes the plasma membrane.

Sequences encoding 7TM domains of Geminigera cryophila CCMP2564, Gene Number CAMMT_002120714; Chroomonas sp., CCMP2293, Gene Number CAMNT_0022112887; Chroomonas sp., CCMP2293, Gene Number CAMNT CAMNT_0022176023; Rhodomonas sp. CCMP768, Gene Number CAMNT_0049533799; Rhodomonas sp. CCMP768, Gene Number CAMNT_0042060367; Rhodomonas sp., Gene Number IAYV-2004853; Rhodomonas sp., Gene Number IAYV-2007142; Rhodomonas sp. CCAC1630), Gene Number KX879689; Rhodomonas sp. CCAC1630, Gene Number KX879690; Proteomonas sulcata CCMP704, Gene Number KX879681; Rhodomonas sp. CCAC1630, Gene Numbers MULTIPLE; Geminigera cryophila CCMP2564, Gene Number CAMNT 0021184197; Geminigera cryophila CCMP2564 Gene Number CAMNT 021188201; Hanusia phi CCMP325, Gene Number CAMNT 0009606213; Hemiselmis andersenii CCMP1180, Gene Number CAMNT 0009587359; Hemiselmis rufescens PCC563, Gene Number CAMNT 0014430495; Proteomonas sulcate CCMP704, Gene Number CAMNT 0026606353; Rhodomonas abbreviate, Gene Number CAMNT 0023412687; Rhodomonas lens RHODO, Gene Number CAMNT 0019250497; Rhodomonas sauna CCMP1319, Gene Number CAMNT 0012765665; Rhodomonas sp. CCMP768, Gene Number CAMNT 0049478653; Rhodomonas sp. CCMP768, Gene Number CAMNT 0049496367; Rhodomonas sp. CCMP768, Gene Number CAMNT 0049531591; Rhodomonas sp. CCMP768, Gene Number CAMNT 0049513053; Rhodomonas sp. CCAC1630, Gene Number IAYV-2051041; and non described CCMP2293, Gene Number CAMNT 0022335561 were optimized for human codon usage and were synthesized. Illumination of neurons expressing such ACRs inhibited their action potential spikes. These ACRs provide new membrane-hyperpolarizing tools for use in establishing a high level of membrane potential for use as optogenetic tools for neuronal silencing of excited cells for among others, neuronal or neurologic disorders, such as but not limited to Parkinson's disease and epilepsy, as well as for cardiac disorders (as described for example by the inventors in Govorunova, E. G., Cunha, C. R., Sineshchekov, 0. A. & Spudich, J. L. Anion channelrhodopsins for inhibitory cardiac optogenetics. Sci. Rep. 6, 33530, 2016, incorporated herein by reference).

In some embodiments herein is disclosed a recombinant nucleic acid operatively linked to a heterologous promoter sequence, said recombinant nucleic acid comprising: a sequence that encodes a polypeptide with at least 85%, 90%, 91%, 92%, 93%, 94%, 95%, 96%, 97%, 98% or 99% identical to a sequence selected from the group consisting of SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118; or a sequence that encodes a polypeptide comprising at least 225, 230, 235, 240, 255, 260, 265, 270, 275, 280, 285, 290, 295, 300, 305, 310, 315 or 320 contiguous amino acids from SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118, or a sequence that hybridizes to the nucleotide sequence of SEQ ID NO: 2, SEQ ID NO: 4, SEQ ID NO: 6, SEQ ID NO:8, SEQ ID NO: 10-46, SEQ ID NO: 48, SEQ ID NO: 50-55 or SEQ ID NO: 70-97 or the complement thereof. In another embodiment, the recombinant nucleic acid comprises an expression vector. In further embodiments, the recombinant nucleic acid that hybridizes to the nucleotide sequence of SEQ ID NO: 2, SEQ ID NO: 4, SEQ ID NO: 6, SEQ ID NO:8, SEQ ID NO: 10-46, SEQ ID NO: 48, SEQ ID NO: 50-55 or SEQ ID NO: 70-97 further comprises hybridization to filter-bound DNA in 0.5 M NaHPO4, 7% sodium dodecyl sulfate, 1 mM EDTA at 65° C., and washing in 0.2×SSC/0.1% SDS at 42° C. In a further embodiment, there is provided a recombinant host cell comprising a recombinant nucleic acid operatively linked to a heterologous promoter sequence, said recombinant nucleic acid comprising: a sequence that encodes a polypeptide with at least 85%, 90%, 91%, 92%, 93%, 94%, 95%, 96%, 97%, 98% or 99% identity to an amino acid sequence selected from SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118 or a sequence that encodes a polypeptide comprising at least 225, 230, 235, 240, 255, 260, 265, 270, 275, 280, 285, 290, 295, 300, 305, 310, 315 or 320 contiguous amino acids from SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118 or a sequence that hybridizes to the nucleotide sequence of SEQ ID NO: 2, SEQ ID NO: 4, SEQ ID NO: 6, SEQ ID NO:8, SEQ ID NO: 10-46, SEQ ID NO: 48, SEQ ID NO: 50-55 or SEQ ID NO: 70-97, or the complement thereof.

In some embodiments, the host cell is an isolated human cell, a non-human mammalian cell, a bacterial cell, a yeast cell, an insect cell, or a plant cell.

In some embodiments, there is provided a method of restoring photosensitivity to a retina of a subject suffering from vision loss or blindness, said method comprising: delivering to the OFF-bipolar neurons of the retina of said subject an expression vector comprising a polynucleotide that encodes: an amino acid sequence at least about 85%, 90%, 91%, 92%, 93%, 94%, 95%, 96%, 97%, 98% or 99% identical to a sequence selected from the group consisting of SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118 which encodes a humanized rhodopsin domain of an ACR expressible in a retinal neuron; and expressing said vector in said retinal neuron, wherein the expressed rhodopsin renders said retinal neuron photosensitive, thereby restoring photosensitivity to enable light-induced silencing of such neuron in said retina or a portion thereof. In a further embodiment, the subject is mammalian, and in a still further embodiment, the subject is human. In some aspects, the method comprises delivering to the retina of said subject an expression vector. In certain aspects, the delivering comprises a pharmaceutically acceptable carrying agent.

In some further embodiments, there is provided an isolated nucleic acid molecule comprising a sequence encoding a rhodopsin domain of an ACR having a sequence at least about 85%, 90%, 91%, 92%, 93%, 94%, 95%, 96%, 97%, 98% or 99% identical to a sequence according to SEQ ID NO: 2, SEQ ID NO: 4, SEQ ID NO: 6, SEQ ID NO:8, SEQ ID NO: 10-46, SEQ ID NO: 48, SEQ ID NO: 50-55 or SEQ ID NO: 70-97, In certain aspects, the isolated nucleic acid molecule comprises a sequence that hybridized to the nucleotide sequence of one of SEQ ID NO: 2, SEQ ID NO: 4, SEQ ID NO: 6, SEQ ID NO:8, SEQ ID NO: 10-46, SEQ ID NO: 48, SEQ ID NO: 50-55 or SEQ ID NO: 70-97, under stringent conditions comprising hybridization to filter-bound DNA in 0.5 M NaHPO₄, 7% sodium dodecyl sulfate, 1 mM EDTA at 65° C., and washing in 0.2×SSC/0.1% SDS at 42° C. and encodes an anion-conducting channelrhodopsin. In some aspects, the nucleic acid is a DNA. In other aspects, the nucleic acid is a RNA (e.g., mRNA). In further embodiments, there is provided an expression vector comprising a nucleic acid molecule provided herein, such as a sequence at least about 90% identical to a sequence according to SEQ ID NO: 2, SEQ ID NO: 4, SEQ ID NO: 6, SEQ ID NO:8, SEQ ID NO: 10-46, SEQ ID NO: 48, SEQ ID NO: 50-55 or SEQ ID NO: 70-97.

In an even further embodiment, there is provided a recombinant host cell comprising a nucleic acid provided herein (e.g., a sequence at least about 90% identical to a sequence according to SEQ ID NO: 2, SEQ ID NO: 4, SEQ ID NO: 6, SEQ ID NO:8, SEQ ID NO: 10-46, SEQ ID NO: 48, SEQ ID NO: 50-55 or SEQ ID NO: 70-97). In some aspects, the host cell is an isolated human cell. In other aspects, the host cell is a non-human mammalian cell. In some aspects, the host cell is a bacterial cell. In certain aspects, the host cell is a yeast cell. In other aspects, the host cell is an insect cell. In some aspects, the host cell is a plant cell. In certain aspects, host cell is an isolated neuronal cell. In particular, the host cell is an isolated electrically active cell.

In another embodiment, there is provided a method of treating a subject suffering from a disorder that involves electrically active cells comprising expressing in the subject an effective amount of a sequence encoding a rhodopsin domain of an ACR anion-conducting channelrhodopsin at the site of the electrically active cells. In some aspects, the subject is suffering from neuropathic pain the method comprising expressing in the subject an effective amount of an anion-conducting channelrhodopsin at the site of the pain. In certain aspects, the subject has an amputated limb, diabetes, multiple sclerosis or has undergone a surgery.

In certain aspects, expressing comprises administering an anion-conducting channelrhodopsin to the subject. In some aspects, the anion-conducting channelrhodopsin comprises an amino acid sequence at least about 85%, 90%, 91%, 92%, 93%, 94%, 95%, 96%, 97%, 98% or 99% identical to a sequence of SEQ ID NO: 1, SEQ ID NO: 3, SEQ ID NO: 5, SEQ ID NO: 7, SEQ ID NO: 9, SEQ ID NO: 47 or SEQ ID NO: 49. In certain aspects, the anion-conducting channelrhodopsin is encoded by a sequence about 90%, 91%, 92%, 93%, 94%, 95%, 96%, 97%, 98% or 99% identical to sequence according to SEQ ID NO: 2, SEQ ID NO: 4, SEQ ID NO: 6, SEQ ID NO:8, SEQ ID NO: 10-46, SEQ ID NO: 48, SEQ ID NO: 50-55 or SEQ ID NO: 70-97. In some aspects, the anion-conducting channelrhodopsin further comprises a cell-penetrating peptide (CPP) sequence or a cellular receptor-binding sequence. As used herein the terms “cell penetrating peptide” refers to segments of polypeptide sequence that allow a polypeptide to cross the cell membrane (e.g., the plasma membrane in the case a eukaryotic cell). Examples of CPP segments include, but are not limited to, segments derived from HIV Tat (e.g., GRKKRRQRRRPPQ (SEQ ID NO: 98)), herpes virus VP22, the Drosophila Antennapedia homeobox gene product, protegrin I, Penetratin (RQIKIWFQNRRMKWKK) or melittin (GIGAVLKVLTTGLPALISWIKRKRQQ (SEQ ID NO: 99)). In certain aspects the CPP comprises the T1 (TKIESLKEHG (SEQ ID NO: 100)), T2 (TQIENLKEKG (SEQ ID NO: 101)), (AALEALAEALEALAEALEALAEAAAA (SEQ ID NO: 102)) or INF7 (GLFEAIEGFIENGWEGMIEGWYGCG (SEQ ID NO: 103)) CPP sequence.

In some aspects, expressing comprises administering a vector encoding an anion-conducting channelrhodopsin to the subject encodes a rhodopsin domain of an ACR. In certain aspects, the vector is a RNA vector. In other aspects, the vector is a DNA vector. In some aspects, the vector is a plasmid, a viral vector or an episomal vector. In certain aspects, the vector further comprises an inducible expression cassette for a suicide gene.

In certain aspects, the sequence encoding the anion-conducting channelrhodopsin encodes a rhodopsin domain of an ACR is operably linked to a heterologous promoter. In some aspects, the promoter is an inducible or a repressible promoter. In certain aspects, the promoter is a tissue or cell type specific promoter. In particular, the promoter is neuronal cell specific promoter.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DESCRIPTION OF ILLUSTRATIVE FIGURES AND DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Action spectra of photocurrents generated in HEK293 cells. An example of a spectrally shifted ACR pair from a single species (C1ACR_023 and C1ACR_887), and an example of three spectrally matching ACRs from another species (R1ACR_367, R1ACR_877 and R1ACR_741). The data points are the mean values±sem (n=4-6 measurements).

FIGS. 2A-D. All functional ACR homologs conduct Cl⁻. (a) Representative series of photocurrent traces recorded from PsuACR_973 (ZipACR) expressed in HEK293 cells in the standard bath (black lines) and upon partial replacement of Cl⁻ with Asp⁻ in the bath. The holding voltage (En) was changed in 20-mV steps from −60 mV at the amplifier output (bottom trace). (b) The current-voltage relationships determined for the experiment shown in panel a. The data points are mean values±sem (n=3 measurements in a typical cell). The data were corrected for liquid junction potentials. (c) The Eᵣ shifts (Eᵣ in the standard bath minus Eᵣ in the Asp bath) measured for all tested ACR homologs and GtACR1 mutants as shown in panel B for PsuACR_973. The data are mean values±sem (n=3-5 cells). The data for the earlier studied GtACRs and PsuACR1 are included for comparison. (d) The Eᵣ shifts (Eᵣ in the standard bath minus Eᵣ in the Asp bath) measured for the other tested ACR homologs R2ACR_853 and R2ACR_142. The data for the earlier studied GtACRs and PsuACR1 are included for comparison.

FIG. 3. Comparison of the mutations introduced to confer Cl− selectivity to CCRs with the corresponding residues in natural ACRs. The numbers on top show the residue numbers according to the CrChR2 sequence.

FIGS. 4A and 4B. ACR functional diversity. The peak current amplitude in response to a 1-s light pulse of the saturating intensity, decay rate and wavelength of maximal sensitivity of cryptophyte ACR homologs. The numbers next to the data points correspond to the protein numbers in Tables 3 and 4. The data were obtained at −60 mV holding potential and standard solutions (see Methods). The data points are the mean values; for sem (n=3-7 cells) see Tables 3 and 4. The data for the previously known GtACR1, GtACR2 and PsuACR1 are included for comparison.

FIG. 5. ZipACR is a fast inhibitory tool for optogenetics. (a) The decay of photocurrents generated by ACRs indicated in the figure legend in HEK293 cells after a 1-s pulse of continuous light of the saturating intensity and the wavelength of the peak absorption of the respective pigment. The traces were normalized at the plateau value measured near the end of the illumination period. (b) Photoinduced spike suppression in cultured rat hippocampal neurons expressing ZipACR (top trace) or GtACR2 (middle trace). Neurons were stimulated by injection of 1-ms current pulses at 50 Hz (represented by the bottom trace). The wavelengths of the 20-ms light pulses of saturating intensity (shown as colored squares) were 520 nm for ZipACR and 470 nm for GtACR2.

FIGS. 6A and 6B. ClustalW protein alignment of seven-helix transmembrane domains of ACR homologs tested in this study. Three previously characterized ACRs are also included. The letters “ACR” are omitted from the names of new proteins. Highlighted are positions in which mutations have been introduced to confer Cl− selectivity to CCRs mutants and those corresponding to the residues that contribute to the central and inner gates in the high-resolution crystal structure 3ug9 of a hybrid CCR (the residue numbers shown above the alignment correspond to the sequence of CrChR2). The boxes show predicted α-helical regions.

FIGS. 7A and 7B. Representative series of photocurrents recorded in response to a 1-s light pulse (shown on top; also serves as a time scale bar) from ACR homologs expressed in HEK293 cells in the standard bath (black lines) and upon partial replacement of Cl− with Asp− in the bath. Note the different scaling of the Y axis in different panels. The holding voltage was changed in 20-mV steps from −60 mV at the amplifier output (bottom trace). Abbreviated protein names are shown in each panel (see Table 1 in the main text for their accession numbers and source organisms).

FIG. 8. ZipACR (PsuACR_973) expression does not affect neuronal morphology. (a) Representative images of cultured rat hippocampal neurons expressing PsuACR_973 (Psu) or control non-transduced neurons (Ctrl). The neurons were stained with antibodies against MAP2 (a dendritic marker), GFP, Synapsin (a synaptic marker) and Tau (an axonal marker). Scale bar 20 μm. (b) The number of primary dendrites per neuron at a 25 μm radius around the center of the soma. (c) The number of synapses per neuron in a 25 μm circle around the center of the soma. The data in panels b & c are the mean values±sem (n=5-12 cells). P values between transduced and control non-transduced neurons (unpaired t test, two-tailed) were >0.5.

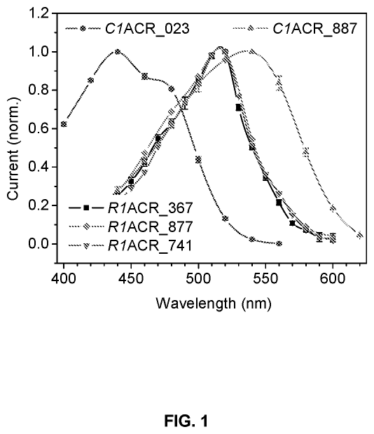

### DESCRIPTION OF ILLUSTRATIVE EMBODIMENTS

Until recently channelrhodopsins have been phototaxis receptors that function as light-gated cation channels that when transfected into animal cells, are used for photoactivation of neuron firing. Described herein are new light gated channels, anion channel rhodopsins (ACRs). Different ACRs and used thereof have been described in, for example, in PCT patent application no: PCT/US2016/023095, incorporated herein by reference. The inventors have now identified a novel set of ACRs that could likewise be used to generate recombinant constructs, such as expression constructs for expressing the ACRs target cells. In some embodiments, the disclosed ACRs can be used in diagnostic and/or therapeutic methods further detailed herein.

### 1. Definitions

In this disclosure, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements and components comprising one unit and elements or components that comprise more than one unit unless specifically stated otherwise.

As used herein, the term “about,” when used in conjunction with a percentage or other numerical amount, means plus or minus 10% of that percentage or other numerical amount. For example, the term “about 80%,” would encompass 80% plus or minus 8%.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls

As used herein, and unless otherwise indicated, the term a disorder that involves electrically active cells, such as but not limited to neuronal dysfunction, a neuron mediated disorder, ocular disorder or cardiac disorder, for which the present methods and compositions may be used include, but are not limited to, neuronal dysfunctions, disorders of the brain, the central nervous system, the peripheral nervous system, neurological conditions, disorders of memory and leaning disorders, cardiac arrhythmias, Parkinson's disease, epilepsy, ocular disorders, spinal cord injury, nerve pain associated with, but not limited to autoimmune diseases (for example, multiple sclerosis, Guillain-Barré syndrome, myasthenia gravis, lupus, and inflammatory bowel disease); cancer and the chemotherapy and radiation used to treat it; compression/trauma (for example, pinched nerves in the neck, crush injuries, and carpal tunnel syndrome); diabetic neuropathy; medication side effects; and toxic substances; motor neuron diseases (for example amyotrophic lateral sclerosis, progressive bulbar palsy, progressive muscular atrophy and primary lateral sclerosis); nutritional deficiencies (for example vitamins B6 and B12); Infectious disease; itch sensations associated with, but not limited to eczema, atopic dermatitis, dry skin and allergic itches; diseases and disorders that alter vagal nerve activity, among others.

As used herein, and unless otherwise indicated, the term ocular disorders for which the present methods and compositions may be used to improve one or more parameters of vision include, but are not limited to, developmental abnormalities that affect both anterior and posterior segments of the eye. Anterior segment disorders include, but are not limited to, glaucoma, cataracts, corneal dystrophy, and keratoconus. Posterior segment disorders include, but are not limited to, blinding disorders caused by photoreceptor malfunction and/or death caused by retinal dystrophies and degenerations. Retinal disorders include congenital stationary night blindness, age-related macular degeneration, congenital cone dystrophies, and a large group of retinitis pigmentosa (RP)-related disorders.

As used herein, and unless otherwise indicated, the terms “treat,” “treating,” “treatment” and “therapy” contemplate an action that occurs while a patient is suffering from a disorder that involves electrically active cells, such as but not limited to neuronal dysfunction, a neuron mediated disorder, ocular disorder or cardiac disorder, and which reduces the severity of one or more symptoms or effect of such a disorder. Where the context allows, the terms “treat,” “treating,” and “treatment” also refers to actions taken toward ensuring that individuals at increased risk of a disorder that involves electrically active cells, such as but not limited to neuronal dysfunction, a neuron mediated disorder, ocular disorder or cardiac disorder and which reduces the severity are able to receive appropriate surgical and/or other medical intervention prior to onset of a disorder that involves electrically active cells, such as but not limited to neuronal dysfunction, a neuron mediated disorder, ocular disorder or cardiac disorder and which reduces the severity. As used herein, and unless otherwise indicated, the terms “prevent,” “preventing,” and “prevention” contemplate an action that occurs before a patient begins to suffer from a disorder that involves electrically active cells, such as but not limited to neuronal dysfunction, a neuron mediated disorder, ocular disorder or cardiac disorder, that delays the onset of, and/or inhibits or reduces the severity of a disorder that involves electrically active cells, such as but not limited to neuronal dysfunction, a neuron mediated disorder, ocular disorder or cardiac disorder.

As used herein, and unless otherwise indicated, the terms “manage,” “managing,” and “management” encompass preventing, delaying, or reducing the severity of a recurrence of a disorder that involves electrically active cells, such as but not limited to neuronal dysfunction, a neuron mediated disorder, ocular disorder or cardiac disorder in a patient who has already suffered from such a disease, disorder or condition. The terms encompass modulating the threshold, development, and/or duration of the disorder that involves electrically active cells or changing how a patient responds to the disorder that involves electrically active cells or the maintenance and/or establishment of a desirable membrane potential across the membrane of a cell.

As used herein, and unless otherwise specified, a “therapeutically effective amount” of a compound is an amount sufficient to provide any therapeutic benefit in the treatment or management of a disorder that involves electrically active cells, such as but not limited to neuronal dysfunction, a neuron mediated disorder, ocular disorder or cardiac disorder, or to delay or minimize one or more symptoms associated with a disorder that involves electrically active cells, such as but not limited to neuronal dysfunction, a neuron mediated disorder, ocular disorder or cardiac disorder. A therapeutically effective amount of a compound means an amount of the compound, alone or in combination with one or more other therapies and/or therapeutic agents that provide any therapeutic benefit in the treatment or management of a disorder that involves electrically active cells, such as but not limited to neuronal dysfunction, a neuron mediated disorder, ocular disorder or cardiac disorder.

The term “therapeutically effective amount” can encompass an amount that alleviates a disorder that involves electrically active cells, such as but not limited to neuronal dysfunction, a neuron mediated disorder, ocular disorder or cardiac disorder, improves or reduces a disorder that involves electrically active cells or improves overall therapy, or enhances the therapeutic efficacy of another therapeutic agent.

As used herein, and unless otherwise specified, a “prophylactically effective amount” of a compound is an amount sufficient to prevent or delay the onset of a disorder that involves electrically active cells, such as but not limited to neuronal dysfunction, a neuron mediated disorder, ocular disorder or cardiac disorder or one or more symptoms associated with a disorder that involves electrically active cells or prevent or delay its recurrence. A prophylactically effective amount of a compound means an amount of the compound, alone or in combination with one or more other treatment and/or prophylactic agent that provides a prophylactic benefit in the prevention of a disorder that involves electrically active cells, such as but not limited to neuronal dysfunction, a neuron mediated disorder, ocular disorder or cardiac disorder. The term “prophylactically effective amount” can encompass an amount that prevents a disorder that involves electrically active cells, such as but not limited to neuronal dysfunction, a neuron mediated disorder, ocular disorder or cardiac disorder, improves overall prophylaxis, or enhances the prophylactic efficacy of another prophylactic agent. The “prophylactically effective amount” can be prescribed prior to, for example, the development of a disorder that involves electrically active cells, such as but not limited to neuronal dysfunction, a neuron mediated disorder, ocular disorder or cardiac disorder.

As used herein, “patient” or “subject” includes mammalian organisms which are capable of suffering from a disorder that involves electrically active cells, such as but not limited to neuronal dysfunction, a neuron mediated disorder, ocular disorder or cardiac disorder, as described herein, such as human and non-human mammals, for example, but not limited to, rodents, mice, rats, non-human primates, companion animals such as dogs and cats as well as livestock, e.g., sheep, cow, horse, etc.

As used herein, the term “conservative substitution” generally refers to amino acid replacements that preserve the structure and functional properties of a protein or polypeptide. Such functionally equivalent (conservative substitution) peptide amino acid sequences include, but are not limited to, additions or substitutions of amino acid residues within the amino acid sequences encoded by a nucleotide sequence that result in a silent change, thus producing a functionally equivalent gene product. Conservative amino acid substitutions may be made on the basis of similarity in polarity, charge, solubility, hydrophobicity, hydrophilicity, and/or the amphipathic nature of the residues involved. For example: nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

As used herein, a “redshift” is a shift to longer wavelength. In contrast a “blueshift” would be a shift to shorter wavelength. These terms apply to both light-emitting and light-absorbing objects.

As used herein the phrase “rhodopsin domain” refers to the “rhodopsin fold”, a 7-transmembrane-helix (7TM) structure characteristic of rhodopsins. As used herein, the channelopsin is the apoprotein, while channelrhodopsin is the protein and retinal. As used herein the term “channelrhodopsin” describes retinylidene proteins (rhodopsins) that function as light-gated ion channels.

The percent identity or homology is determined with regard to the length of the relevant amino acid sequence. Therefore, if a polypeptide of the present invention is comprised within a larger polypeptide, the percent homology is determined with regard only to the portion of the polypeptide that corresponds to the polypeptide of the present invention and not the percent homology of the entirety of the larger polypeptide. “Percent identity” or “% identity,” with reference to nucleic acid sequences, refers to the percentage of identical nucleotides between at least two polynucleotide sequences aligned using the Basic Local Alignment Search Tool (BLAST) engine. See Tatusova et al. (1999) FEMS Microbiol Lett. 174:247-250. The BLAST engine is provided to the public by the National Center for Biotechnology Information (NCBI), Bethesda, Md. To align two polynucleotide sequences, the BLAST which employs the “blastn” program is used.

“Percent identity” or “% identity,” with reference to polypeptide sequences, refers to the percentage of identical amino acids between at least two polypeptide sequences aligned using the Basic Local Alignment Search Tool (BLAST) engine. See Tatusova et al. (1999) ibid. The BLAST engine is provided to the public by the National Center for Biotechnology Information (NCBI), Bethesda, Md. To align two polypeptide sequences, the BLAST which employs the “blastp” program is used.

Channelrhodopsins

Sequences encoding 7TM domain channelrhodopsins provided herein. In particular, previously unknown Channelrhodopsins having the amino acid sequence of SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118 or encoded by the polynucleotide sequence of SEQ ID NO: 2, SEQ ID NO: 4, SEQ ID NO: 6, SEQ ID NO:8, SEQ ID NO: 10-46, SEQ ID NO: 48, SEQ ID NO: 50-55 or SEQ ID NO: 70-97 are provided.

In some embodiments, are conserved variants of functional humanized rhodopsin domain or a peptide fragment thereof. A “conservative” amino acid substitution refers to the substitution of an amino acid in a polypeptide with another amino acid having similar properties, such as size or charge. In certain embodiments, a polypeptide comprising a conservative amino acid substitution maintains at least one activity of the unsubstituted polypeptide. A conservative amino acid substitution may encompass non-naturally occurring amino acid residues which are typically incorporated by chemical peptide synthesis rather than by synthesis in biological systems. These include, but are not limited to, peptidomimetics and other reversed or inverted forms of amino acid moieties.

In some embodiments, are any of the disclosed methods, wherein the rhodopsin domain of an anion-conducting channelrhodopsin having the amino acid sequence of all or part of SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118 or a biologically active fragment thereof that retains the biological activity of the encoded rhodopsin domain of an anion-conducting channelrhodopsin or a biologically active conservative amino acid substitution variant of SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118 or of said fragment.

A. Channelrhodopsin Polypeptides

In some embodiments, are isolated polypeptides that encode a rhodopsin domain of an anion-conducting channelrhodopsin. In some embodiments, an isolated polypeptide comprising the amino acid sequence of SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118. In some embodiments, the isolated polypeptide has at least 85% homology to the amino acid sequence of SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118. In some embodiments, the isolated polypeptide has between 85%-95%-100% homology to the amino acid sequence of SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118.

In some embodiments, is a protein composition comprises a polypeptide having the amino acid sequence of SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118. The peptide amino acid sequences that can be used in various embodiments including the anion-conducting rhodopsin domain amino acid sequences described herein (SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118), as well as analogues and derivatives thereof and functional fragments such as but not limited to the rhodopsin/7TM domain. In fact, in some embodiments the any desired peptide amino acid sequences encoded by particular nucleotide sequences can be used, as is the use of any polynucleotide sequences encoding all, or any portion, of desired peptide amino acid sequences. The degenerate nature of the genetic code is well-known, and, accordingly, each anion-conducting rhodopsin domain peptide amino acid-encoding nucleotide sequence is generically representative of the well-known nucleic acid “triplet” codon, or in many cases codons, that can encode the amino acid. As such, as contemplated herein, the anion-conducting channelrhodopsin peptide amino acid sequences described herein, when taken together with the genetic code (see, e.g., “Molecular Cell Biology”, Table 4-1 at page 109 (Darnell et al., eds., W. H. Freeman & Company, New York, N.Y., 1986)), are generically representative of all the various permutations and combinations of nucleic acid sequences that can encode such amino acid sequences.

Such functionally equivalent peptide amino acid sequences (conservative substitutions) include, but are not limited to, additions or substitutions of amino acid residues within the amino acid sequences encoded by a nucleotide sequence, but that result in a silent change, thus producing a functionally equivalent gene product. Amino acid substitutions may be made on the basis of similarity in polarity, charge, solubility, hydrophobicity, hydrophilicity, and/or the amphipathic nature of the residues involved. For example: nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

Naturally occurring residues may be divided into classes based on common side chain properties: hydrophobic (Met, Ala, Val, Leu, Ile); neutral hydrophilic (Cys, Ser, Thr, Asn, Gln); acidic (Asp, Glu); basic (His, Lys, Arg); residues that influence chain orientation (Gly, Pro); and aromatic (Trp, Tyr, Phe). For example, non-conservative substitutions may involve the exchange of a member of one of these classes for a member from another class.

In making substitutions, according to certain embodiments, the hydropathic index of amino acids may be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein, in certain instances, is understood in the art (Kyte et al., J. Mol. Biol., 157:105-131 (1982)). It is known that in certain instances, certain amino acids may be substituted for other amino acids having a similar hydropathic index or score and still retain a similar biological activity. In making changes based upon the hydropathic index, in certain embodiments, the substitution of amino acids whose hydropathic indices are within ±2 is included. In certain embodiments, those which are within ±1 are included, and in certain embodiments, those within ±0.5 are included.

Substitution of like amino acids can be made effectively on the basis of hydrophilicity, particularly where the biologically functional protein or peptide thereby created is intended for use in immunological embodiments, as in the present case. In certain embodiments, the greatest local average hydrophilicity of a protein, as governed by the hydrophilicity of its adjacent amino acids, correlates with its immunogenicity and antigenicity, i.e., with a biological property of the protein.

The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in certain embodiments, the substitution of amino acids whose hydrophilicity values are within ±2 is included, in certain embodiments, those which are within ±1 are included, and in certain embodiments, those within ±0.5 are included.

A skilled artisan will be able to determine suitable variants of a polypeptide as set forth herein using well-known techniques. In certain embodiments, one skilled in the art may identify suitable areas of the molecule that may be changed without destroying activity by targeting regions not believed to be important for activity. In certain embodiments, one can identify residues and portions of the molecules that are conserved among similar polypeptides.

In certain embodiments, even areas that may be important for biological activity or for structure may be subject to conservative amino acid substitutions without destroying the biological activity or without adversely affecting the polypeptide structure.

Additionally, in certain embodiments, one skilled in the art can review structure-function studies identifying residues in similar polypeptides that are important for activity or structure. In view of such a comparison, in certain embodiments, one can predict the importance of amino acid residues in a protein that correspond to amino acid residues which are important for activity or structure in similar proteins. In certain embodiments, one skilled in the art may opt for chemically similar amino acid substitutions for such predicted important amino acid residues.

In certain embodiments, one skilled in the art can also analyze the three-dimensional structure and amino acid sequence in relation to that structure in similar polypeptides. In certain embodiments, in view of such information, one skilled in the art may predict the alignment of amino acid residues of a polypeptide with respect to its three dimensional structure. In certain embodiments, one skilled in the art may choose not to make radical changes to amino acid residues predicted to be on the surface of the protein, since such residues may be involved in important interactions with other molecules.

Moreover, in certain embodiments, one skilled in the art may generate test variants containing a single amino acid substitution at each desired amino acid residue. In certain embodiments, the variants can then be screened using activity assays known to those skilled in the art. In certain embodiments, such variants could be used to gather information about suitable variants. For example, in certain embodiments, if one discovered that a change to a particular amino acid residue resulted in destroyed, undesirably reduced, or unsuitable activity, variants with such a change may be avoided. In other words, in certain embodiments, based on information gathered from such routine experiments, one skilled in the art can readily determine the amino acids where further substitutions should be avoided either alone or in combination with other mutations.

A number of scientific publications have been devoted to the prediction of secondary structure. See, e.g., Moult J., Curr. Op. in Biotech., 7(4):422-427 (1996), Chou et al., Biochemistry, 13(2):222-245 (1974); Chou et al., Biochemistry, 113(2):211-222 (1974); Chou et al., Adv. Enzymol. Relat. Areas Mol. Biol., 47:45-148 (1978); Chou et al., Ann. Rev. Biochem., 47:251-276 and Chou et al., Biophys. J., 26:367-384 (1979). Moreover, computer programs are currently available to assist with predicting secondary structure. One method of predicting secondary structure is based upon homology modeling. For example, two polypeptides or proteins which have a sequence identity of greater than 30%, or similarity greater than 40% often have similar structural topologies. The growth of the protein structural database (PDB) has provided enhanced predictability of secondary structure, including the potential number of folds within a polypeptide's structure. See, e.g., Holm et al., Nucl. Acid. Res., 27(1):244-247 (1999). It has been suggested (Brenner et al., Curr. Op. Struct. Biol., 7(3):369-376 (1997)) that there are a limited number of folds in a given polypeptide or protein and that once a critical number of structures have been resolved, structural prediction will become dramatically more accurate.

Additional methods of predicting secondary structure include “threading” (see, e.g., Jones, D., Curr. Opin. Struct. Biol., 7(3):377-87 (1997); Sippl et al., Structure, 4(1):15-19 (1996)), “profile analysis” (see, e.g., Bowie et al., Science, 253:164-170 (1991); Gribskov et al., Meth. Enzym., 183:146-159 (1990); Gribskov et al., Proc. Nat. Acad. Sci., 84(13):4355-4358 (1987)), and “evolutionary linkage” (see, e.g., Holm et al., Nucl. Acid. Res., 27(1):244-247 (1999), and Brenner et al., Curr. Op. Struct. Biol., 7(3):369-376 (1997)).

In certain embodiments, a variant of the reference channelrhodopsin or rhodopsin domain, such as those encoded by SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118 includes a glycosylation variant wherein the number and/or type of glycosylation sites have been altered relative to the amino acid sequence of the reference anion-conducting channelrhodopsin or rhodopsin domain. In certain embodiments, a variant of a polypeptide comprises a greater or a lesser number of N-linked glycosylation sites relative to a native polypeptide. An N-linked glycosylation site is characterized by the sequence: Asn-X-Ser or Asn-X-Thr, wherein the amino acid residue designated as X may be any amino acid residue except proline. The substitution of amino acid residues to create this sequence provides a potential new site for the addition of an N-linked carbohydrate chain. Alternatively, substitutions which eliminate this sequence will remove an existing N-linked carbohydrate chain. In certain embodiments, a rearrangement of N-linked carbohydrate chains is provided, wherein one or more N-linked glycosylation sites (typically those that are naturally occurring) are eliminated and one or more new N-linked sites are created. Exemplary variants include cysteine variants wherein one or more cysteine residues are deleted from or substituted for another amino acid (e.g., serine) relative to the amino acid sequence of the reference channelrhodopsin or rhodopsin domain (e.g., such as those provided as SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118). In certain embodiments, cysteine variants may be useful when polypeptides and proteins must be refolded into a biologically active conformation such as after the isolation of insoluble inclusion bodies. In certain embodiments, cysteine variants have fewer cysteine residues than the native polypeptide. In certain embodiments, cysteine variants have an even number of cysteine residues to minimize interactions resulting from unpaired cysteines.

According to certain embodiments, amino acid substitutions are those which: (1) reduce susceptibility to proteolysis, (2) reduce susceptibility to oxidation, (3) alter binding affinity for forming protein complexes, (4) alter binding affinities, and/or (4) confer or modify other physiochemical or functional properties on such polypeptides. According to certain embodiments, single or multiple amino acid substitutions (in certain embodiments, conservative amino acid substitutions) may be made in a naturally-occurring sequence (in certain embodiments, in the portion of the polypeptide outside the domain(s) forming intermolecular contacts). In certain embodiments, a conservative amino acid substitution typically may not substantially change the structural characteristics of the reference sequence (e.g., in certain embodiments, a replacement amino acid should not tend to break a helix that occurs in the reference sequence, or disrupt other types of secondary structure that characterizes the reference sequence).

Examples of certain art-recognized polypeptide secondary and tertiary structures are described, for example, in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et at. Nature 354:105 (1991).

In other embodiments, are methods and compositions that provide an ACR with improved properties and characteristics that enhance the application of the compositions in, among other things, optogenetic techniques. Improved properties include, but are not limited to adaptation to human codon usage and synthesis. These embodiments provide greater sensitivity and efficient membrane hyperpolarization and neuronal silencing through light-gated chloride conduction or in the case of neurons with high cytoplasmic chloride concentration, wherein the expressed rhodopsin depolarizes the plasma membrane.

B. Fusion Proteins

The use of fusion proteins in which a polypeptide or peptide, or a truncated or mutant version of peptide is fused to an unrelated or homologous protein, polypeptide, or peptide, and can be designed on the basis of the desired peptide encoding nucleic acid and/or amino acid sequences described herein. Such fusion proteins include, but are not limited to: IgFc fusions, which stabilize proteins or peptides and prolong half-life in vivo; fusions to any amino acid sequence that allows the fusion protein to be anchored to the cell membrane; or fusions to an enzyme, fluorescent protein, or luminescent protein that provides a marker function. Fusion proteins to homologous proteins include, but are not limited to, those that are produced from genes that are engineered to encode a portion of the anion-conducting channelrhodopsin fused to a portion of a homologous (orthologous or paralogous) protein of the same of related function. For example, chimeras between different channelrhodopsins may be made to combine beneficial properties uniquely present in each. In some aspects, a chimeric channelrhodopsin of the embodiments comprises about 50%, 55%, 60%, 65%, 70%, 75%, 80%, 85%, 90% or 95% of its sequence from a first channelrhodopsin and the remaining sequence from a second channelrhodopsin. In some aspects, a chimeric channelrhodopsin comprises the rhodopsin domain of a first channelrhodopsin and the remaining sequence from a second channelrhodopsin. In yet further aspects, a chimeric channelrhodopsin can comprise 1, 2, 3, 4, 5 or 6 of its transmembrane domains from a first channelrhodopsin and the remaining transmembrane domains from a second channelrhodopsin.

In certain embodiments, a fusion protein may be readily purified by utilizing an antibody that selectively binds to the fusion protein being expressed. In alternate embodiments, a fusion protein may be purified by subcloning peptide encoding nucleic acid sequence into a recombination plasmid, or a portion thereof, is translationally fused to an amino-terminal (N-terminal) or carboxy-terminal (C-terminal) tag consisting of six histidine residues (a “His-tag”; see, e.g., Janknecht et al., Proc. Natl. Acad. Sci. USA 88:8972-8976, 1991). Extracts from cells expressing such a construct are loaded onto Ni²⁺ nitriloacetic acid-agarose columns, and histidine-tagged proteins are selectively eluted with imidazole-containing buffers.

C. Nucleic Acids Encoding Channelrhodopsins

In some embodiments, a recombinant nucleic acid operatively linked to a heterologous promoter sequence, said recombinant nucleic acid comprising: a sequence that encodes a peptide with at least 85% homology to an amino acid sequence selected from SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118 or a sequence that encodes a peptide comprising 225 contiguous amino acids selected from SEQ ID NO: 1, SEQ ID NO: 3, SEQ ID NO: 5, SEQ ID NO: 7, SEQ ID NO: 9, SEQ ID NO: 47 or SEQ ID NO: 49; or a sequence that hybridizes to the nucleotide sequence of SEQ ID NO: 2, SEQ ID NO: 4, SEQ ID NO: 6, SEQ ID NO:8, SEQ ID NO: 10-46, SEQ ID NO: 48, SEQ ID NO: 50-55 or SEQ ID NO: 70-97 or the complement thereof.

In some embodiments, are isolated nucleic acid molecules comprising a nucleotide sequence that encode an anion-conducting channelrhodopsins derived from algae. In some embodiments, the rhodopsin domain encodes the peptides whose sequence is described in SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118. In some embodiments, are isolated nucleic acid molecules comprising a nucleotide sequence that encodes the amino acid sequence shown in SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118. In some embodiments, are expression vectors comprising a nucleic acid sequence that encodes the amino acid sequences of SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118. In some embodiments, are host cells comprising an expression vector comprising a nucleic acid sequence that encodes the amino acid sequences of SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118 or a nucleic acid sequence, fragment of portion thereof of the nucleic acid sequences of SEQ ID NO: 2, SEQ ID NO: 4, SEQ ID NO: 6, SEQ ID NO:8, SEQ ID NO: 10-46, SEQ ID NO: 48, SEQ ID NO: 50-55 or SEQ ID NO: 70-97.

In some embodiments, are isolated nucleic acid molecules comprising a nucleotide sequence that was derived from cDNA and encode the rhodopsin domain of an ACR. In some embodiments, the rhodopsin domain encodes the peptides whose sequence is described in SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118. In some embodiments, are isolated nucleic acid molecules that were derived from cDNA that comprise a nucleotide sequence that encodes the amino acid sequence shown in SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118. In some embodiments, are expression vectors comprising a nucleic acid sequence that encodes the amino acid sequences of SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118. In some embodiments, are host cells comprising a recombinant expression vector comprising a nucleic acid sequence that was derived from cDNA and encode the amino acid sequences of SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118.

In some embodiments are isolated peptides comprising an amino acid sequence encoded by at least a portion of the cDNA derived nucleic acid sequences that encode the 7TM or rhodopsin domain of an ACR. In some embodiments, are isolated peptides comprising an amino acid sequence encoded by a cDNA derived nucleic acid sequence that encodes an amino acid sequence of SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118

In some embodiments are isolated peptides comprising a contiguous sequence encoded by a nucleic acid sequence that encodes the anion rhodopsin domain of a anion-conducting channelrhodopsin derived. In some embodiments, are isolated peptides comprising an amino acid sequence of SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118, or fragment thereof. In some embodiments, are isolated peptides comprising an amino acid sequence encoded by at least a portion of a nucleic acid sequence of a group consisting of SEQ ID NO: 2, SEQ ID NO: 4, SEQ ID NO: 6, SEQ ID NO:8, SEQ ID NO: 10-46, SEQ ID NO: 48, SEQ ID NO: 50-55 or SEQ ID NO: 70-97 and which functions as a anion rhodopsin or anion-conducting channelrhodopsin.

In some embodiments, are isolated peptides comprising an amino acid sequence of SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118 or a 7 TM domain/rhodopsin domain encoded by a cDNA derived nucleic acid sequence of SEQ ID NO: 2, SEQ ID NO: 4, SEQ ID NO: 6, SEQ ID NO:8, SEQ ID NO: 10-46, SEQ ID NO: 48, SEQ ID NO: 50-55 or SEQ ID NO: 70-97 and which functions as an anion-conducting channelrhodopsin.

In some embodiments, isolated nucleic acid molecules are provided comprising a nucleotide sequence that encodes the rhodopsin domain of a anion-conducting channelrhodopsin. In some embodiments, the rhodopsin encodes a peptide whose sequence is shown in SEQ ID NO: 1, SEQ ID NO: 3, SEQ ID NO: 5, SEQ ID NO: 7, SEQ ID NO: 9, SEQ ID NO: 47 or SEQ ID NO: 49. In some embodiments, are isolated nucleic acid molecules comprising a nucleotide sequence that encodes the amino acid sequence shown in SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118. In some embodiments, are expression vectors comprising a nucleic acid sequence that encodes the amino acid sequences of SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118. In some embodiments, are host cells comprising a expression vector comprising a nucleic acid sequence that encodes the amino acid sequences of SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118. In some embodiments, are peptides comprising a sequence that encodes the rhodopsin domain of an anion-conducting channelrhodopsin. In some embodiments, are isolated peptides comprising an amino acid sequence of SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118. In some embodiments, the isolated peptides comprise an amino acid sequence of SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118, or fragments thereof.

In some embodiments, are isolated nucleic acid molecules wherein said nucleic acid molecule has a sequence of SEQ ID NO: 2, SEQ ID NO: 4, SEQ ID NO: 6, SEQ ID NO:8, SEQ ID NO: 10-46, SEQ ID NO: 48, SEQ ID NO: 50-55 or SEQ ID NO: 70-97. In other embodiments, are expression vectors comprising a nucleic acid sequence selected from that shown in SEQ ID NO: 2, SEQ ID NO: 4, SEQ ID NO: 6, SEQ ID NO:8, SEQ ID NO: 10-46, SEQ ID NO: 48, SEQ ID NO: 50-55 or SEQ ID NO: 70-97 and those that encode the amino acid sequences shown in SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118. In some embodiments, are host cells comprising a expression vector comprising a nucleic acid sequence shown in SEQ ID NO: 2, SEQ ID NO: 4, SEQ ID NO: 6, SEQ ID NO:8, SEQ ID NO: 10-46, SEQ ID NO: 48, SEQ ID NO: 50-55 or SEQ ID NO: 70-97 and those that encode the amino acid sequences of SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118. In some embodiments, an isolated nucleic acid comprises a nucleotide sequence that encodes the rhodopsin domain of an anion-conducting channelrhodopsin. In some embodiments, the nucleotide sequence encodes at least 16, 20, 33, 35, or 75 contiguous amino acids of SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118. In some embodiments, the nucleotide sequence encodes a peptide comprising any contiguous portion of SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118.

In some embodiments, an isolated nucleic acid comprising a nucleotide sequence that encodes a functional domain of an anion-conducting channelrhodopsin. In some embodiments are isolated nucleic acid that encodes at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 75, 100, 125, 150, 175, 200, 205, 210, 215, 220, 225, 228, 229, 230, 235, 240, 250, 255, 260, 265, 270, 275, 280, 285, 290, 295, 296 or more contiguous amino acids of SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118. Further, in some embodiments, any range derivable between any of the above-described integers.

In other embodiments, there is provided an isolated polypeptide or an isolated nucleic acid encoding a polypeptide having in some embodiments between about 70% and about 75%; in further embodiments between about 75% and about 80%; in further still embodiments between about 80% and 90%; or even more further between about 90% and about 99% of amino acids (for example 95%) that are identical to (or homologous to) the amino acids of SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118 or fragments thereof.

In other embodiments, the present invention provides for an isolated nucleic acid encoding a polypeptide having between about 70% and about 75%; or more preferably between about 75% and about 80%; or more preferably between about 80% and 90%; or even more preferably between about 90% and about 99% of amino acids that are identical to the amino acids of SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118 or fragments thereof.

In some embodiments, the nucleic acid segments, regardless of the length of the coding sequence itself, may be combined with other DNA sequences, such as promoters, enhancers, polyadenylation signals, additional restriction enzyme sites, multiple cloning sites, other coding segments, and the like. In some embodiments, for example, are recombinant nucleic acids comprising a nucleotide sequence that encodes amino acids of SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118 or fragments thereof, operably linked to a heterologous promoter.

In certain embodiments the invention provides an isolated nucleic acid obtained by amplification from a template nucleic acid using a primer selected from appropriate primer that can be used with SEQ ID NO: 2, SEQ ID NO: 4, SEQ ID NO: 6, SEQ ID NO:8, SEQ ID NO: 10-46, SEQ ID NO: 48, SEQ ID NO: 50-55 or SEQ ID NO: 70-97.

In some embodiments, are any of the disclosed methods wherein the expression vectors include, but are not limited to, AAV viral vector. In some embodiments, are any of the disclosed methods wherein the promoter is a constitutive promoter. In some embodiments, are any of the disclosed methods wherein the constitutive promoter includes, but is not limited to, a CMV promoter or a hybrid CMV enhancer/chicken β-actin (CAG) promoter. In some embodiments, are any of the disclosed methods wherein the promoter includes, but is not limited to, an inducible and/or a cell type-specific promoter.

In some embodiments, there is provided a cDNA-derived nucleic acid comprising a nucleic acid sequence that encodes an amino acid sequence selected from SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118 wherein the cDNA-derived nucleic acid comprises the nucleic acid sequence of SEQ ID NO: 2, SEQ ID NO: 4, SEQ ID NO: 6, SEQ ID NO:8, SEQ ID NO: 10-46, SEQ ID NO: 48, SEQ ID NO: 50-55 or SEQ ID NO: 70-97. In other embodiments there is an expression vector comprising the cDNA-derived nucleic acid comprising a nucleic acid sequence that encodes an amino acid sequence selected from SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118.

Rhodopsin domain nucleic acid sequences for use in the disclosed methods and compositions include, but are not limited to, the active portion of the rhodopsin domains presently disclosed and encoded by the nucleic acid sequences of SEQ ID NO: 2, SEQ ID NO: 4, SEQ ID NO: 6, SEQ ID NO:8, SEQ ID NO: 10-46, SEQ ID NO: 48, SEQ ID NO: 50-55 or SEQ ID NO: 70-97.

In some embodiments, the use of an active portion of a presently disclosed anion-conducting channelrhodopsin, such as but not limited to the rhodopsin domain, includes all or portions of the sequences described herein (and expression vectors comprising the same), and additionally contemplates the use of any nucleotide sequence encoding a contiguous active portion of the presently disclosed anion-conducting channelrhodopsins, such as but not limited to the rhodopsin domain, open reading frame (ORF) that hybridizes to a complement of an anion-conducting channelrhodopsin or channelopsin sequence described herein under highly stringent conditions, e.g., hybridization to filter-bound DNA in 0.5 M NaHPO₄, 7% sodium dodecyl sulfate (SDS), 1 mM EDTA at 65° C., and washing in 0.1×SSC/0.1% SDS at 68° C. (“Current Protocols in Molecular Biology”, Vol. 1 and 2 (Ausubel et al., eds., Green Publishing Associates, Incorporated, and John Wiley & Sons, Incorporated, New York, N.Y., 1989)), and encodes a functionally equivalent anion-conducting channelrhodopsin (or active portion thereof, such as but not limited to the rhodopsin domain) gene product or the active portion thereof. Additionally contemplated is the use of any nucleotide sequence that hybridizes to the complement of a DNA sequence that encodes an anion-conducting channelrhodopsin amino acid sequence under moderately stringent conditions, e.g., washing in 0.2×SSC/0.1% SDS at 42° C. (“Current Protocols in Molecular Biology”, supra), yet still encodes a functionally equivalent anion-conducting channelrhodopsin product. Functional equivalents of anion-conducting channelrhodopsin include, but are not limited to, naturally occurring versions of anion-conducting channelrhodopsin present in other or the same species (orthologs, paralogs and more generally homologs), and mutant versions of anion-conducting channelrhodopsin, whether naturally occurring or engineered (by site directed mutagenesis, gene shuffling, or directed evolution, as described in, for example, U.S. Pat. No. 5,837,458) or active portion thereof, such as but not limited to the rhodopsin domain. The disclosure also includes the use of degenerate nucleic acid variants (due to the redundancy of the genetic code) of the identified channelrhodopsin polynucleotide sequences.

Additionally contemplated is the use of polynucleotides encoding anion-conducting channelrhodopsin ORFs, or their functional equivalents, encoded by polynucleotide sequences that are about 99, 95, 90, or about 85 percent similar to the corresponding regions of the anion-conducting channelrhodopsin sequences described herein (as measured by BLAST sequence comparison analysis using, for example, the University of Wisconsin GCG sequence analysis package (SEQUENCHER 3.0, Gene Codes Corporation, Ann Arbor, Mich.) using default parameters).

The nucleic acid segments of the embodiments, regardless of the length of the coding sequence itself, may be combined with other sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol. For example, nucleic acid fragments may be prepared which include a short stretch complementary to nucleic acids that encode the polypeptides of SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118, such as about 10 to 15 or 20, 30, or 40 or so nucleotides, and which are up to 2000 or so base pairs in length. DNA segments with total lengths of about 8000, 7000, 6000, 5000, 4000, 3000, 2000, 1000, 500, 200, 100 and about 50 base pairs in length are also contemplated to be useful.

In some embodiments, isolated nucleic acids that encode the amino acids of a channelrhodopsin or fragment thereof and recombinant vectors incorporating nucleic acid sequences which encode a channelrhodopsin protein or peptide and that includes within its amino acid sequence an amino acid sequence in accordance with SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118. In some embodiments, a purified nucleic acid segment that encodes a protein that encodes a channelrhodopsin or fragment thereof, the recombinant vector may be further defined as an expression vector comprising a promoter operatively linked to said channelrhodopsin-encoding nucleic acid segment.

In additional embodiments, is a host cell, made recombinant with a recombinant vector comprising channelrhodopsin-encoding nucleic acid segments. The recombinant host cell may be a prokaryotic cell or a eukaryotic cell. As used herein, the term “engineered” or “recombinant” cell is intended to refer to a cell into which a recombinant gene, such as a gene encoding a channelrhodopsin, has been introduced. Therefore, engineered cells are distinguishable from naturally occurring cells which do not contain a recombinantly introduced gene. Engineered cells are thus cells having a gene or genes introduced through the hand of man. Recombinantly introduced genes will either be in the form of a copy of a genomic gene or a cDNA gene, or will include genes positioned adjacent to a promoter not naturally associated with the particular introduced gene. In some embodiments, nucleic acid molecules having sequence regions consisting of contiguous nucleotide stretches of about 14, 15-20, 30, 40, 50, or even of about 100 to about 200 nucleotides or so, identical or complementary to the channelrhodopsin-encoding nucleic acid sequences.

In still further aspects, there is provided a host cell comprising a nucleic acid molecule of the embodiments (e.g., that encodes an ACR). Thus, in some embodiments a host cell is an: isolated human cell; a non-human mammalian cell; a bacterial cell; a yeast cell; an insect cell; or a plant cell. In alternative embodiments, the nucleic acid sequences described can be targeted to the genome of a cell using a CRISPR-associated protein-9 nuclease (Cas9) based system for genome-editing and genome targeting. In some embodiments, delivery to some cells may require delivery systems, such as, but not limited to those based on lentivirus (LVs), adenovirus (AdV) and adenoassociated (AAV).

An exemplary DNA binding protein is an RNA guided DNA binding protein of a Type II CRISPR System. An exemplary DNA binding protein is a Cas9 protein. According to one aspect, an engineered Cas9-gRNA system is provided which enables RNA-guided genome cutting in a site specific manner, if desired, and modification of the genome by insertion of exogenous channelrhodopsin-encoding nucleic acids provided herein. The guide RNAs are complementary to target sites or target loci on the DNA. The guide RNAs can be crRNA-tracrRNA chimeras. The Cas9 binds at or near target genomic DNA. The one or more guide RNAs bind at or near target genomic DNA. The Cas9 cuts the target genomic DNA and exogenous donor DNA is inserted into the DNA at the cut site.

Accordingly, methods are directed to the use of a guide RNA with a Cas9 protein and an exogenous channelrhodopsin-encoding nucleic acid to multiplex insertions of exogenous channelrhodopsin-encoding nucleic acids into DNA within a cell expressing Cas9 by cycling the insertion of nucleic acid encoding the RNA and exogenous donor nucleic acid, expressing the RNA, colocalizing the RNA, Cas9 and DNA in a manner to cut the DNA, and insertion of the exogenous donor nucleic acid. The method steps can be cycled in any desired number to result in any desired number of DNA modifications.

D. Recombinant Expression

While the desired peptide amino acid sequences described can be chemically synthesized (see, e.g., “Proteins: Structures and Molecular Principles” (Creighton, ed., W. H. Freeman & Company, New York, N.Y., 1984)), large polypeptides sequences may advantageously be produced by recombinant DNA technology using techniques well-known in the art for expressing nucleic acids containing a nucleic acid sequence that encodes the desired peptide. Such methods can be used to construct expression vectors containing peptide encoding nucleotide sequences and appropriate transcriptional and translational control signals. These methods include, for example, in vitro recombinant DNA techniques, synthetic techniques, and in vivo genetic recombination (see, e.g., “Molecular Cloning, A Laboratory Manual”, supra, and “Current Protocols in Molecular Biology”, supra). Alternatively, RNA and/or DNA encoding desired peptide encoding nucleotide sequences may be chemically synthesized using, for example, synthesizers (see, e.g., “Oligonucleotide Synthesis: A Practical Approach” (Gait, ed., IRL Press, Oxford, United Kingdom, 1984)).

A variety of host-expression vector systems may be utilized to express peptide encoding nucleotide sequences. When the desired peptide or polypeptide is soluble or a soluble derivative, the peptide or polypeptide can be recovered from the host cell culture, i.e., from the host cell in cases where the peptide or polypeptide is not secreted, and from the culture media in cases where the peptide or polypeptide is secreted by the host cell. However, suitable expression systems also encompass engineered host cells that express the desired polypeptide or functional equivalents anchored in the cell membrane. Purification or enrichment of the desired peptide from such expression systems can be accomplished using appropriate detergents and lipid micelles, and methods well-known to those skilled in the art. Furthermore, such engineered host cells themselves may be used in situations where it is desired not only to retain the structural and functional characteristics of the peptide, but to assess biological activity, e.g., in certain drug screening assays.

In certain applications, transient expression systems are desired. However, for long-term, high-yield production of recombinant proteins or peptides, stable expression is generally preferred. For example, cell lines that stably express the desired protein, polypeptide, peptide, or fusion protein may be engineered. Rather than using expression vectors that contain viral origins of replication, host cells can be transformed with DNA controlled by appropriate expression control elements (e.g., promoter, enhancer sequences, transcription terminators, polyadenylation sites, etc.), and a selectable marker. Following the introduction of the foreign DNA, engineered cells are allowed to grow for about 1-2 days in an enriched media, and then switched to a selective media. The selectable marker in the recombinant plasmid confers resistance to the selection, and allows cells to stably integrate the plasmid into their chromosomes and grow to form foci, which in turn can be cloned and expanded into cell lines. This method may advantageously be used to engineer cell lines that express the desired gene products or portions thereof. Such engineered cell lines may be particularly useful in screening and evaluation of compounds that affect the endogenous activity of a desired protein, polypeptide or peptide.

A number of selection systems may be used, including, but not limited to, the herpes simplex virus thymidine kinase (Wigler et al., Cell 11:223-232, 1977), hypoxanthine-guanine phosphoribosyltransferase (Szybalska and Szybalski, Proc. Natl. Acad. Sci. USA 48:2026-2034, 1962), and adenine phosphoribosyltransferase (Lowy et al., Cell 22:817-823, 1980) genes, which can be employed in tk⁻, hgprt⁻ or aprt⁻ cells, respectively. Anti-metabolite resistance can also be used as the basis of selection for the following genes: dihydrofolate reductase (dhfr), which confers resistance to methotrexate (Wigler et al., Proc. Natl. Acad. Sci. USA 77:3567-3570, 1980, and O'Hare et al., Proc. Natl. Acad. Sci. USA 78:1527-1531, 1981); guanine phosphoribosyl transferase (gpt), which confers resistance to mycophenolic acid (Mulligan and Berg, Proc. Natl. Acad. Sci. USA 78:2072-2076, 1981); neomycin phosphotransferase (neo), which confers resistance to the aminoglycoside G-418 (Colbere-Garapin et al., J. Mol. Biol. 150:1-14, 1981); and hygromycin B phosphotransferase (hpt), which confers resistance to hygromycin (Santerre et al., Gene 30:147-156, 1984).

Host cells/expression systems that may be used for purpose of providing compositions to be used in the disclosed methods include, but are not limited to, microorganisms such as bacteria (e.g., E. coli, B. subtilis) transformed with a recombinant bacteriophage DNA, plasmid DNA, or cosmid DNA expression vector containing a desired peptide encoding nucleotide sequence; yeast (e.g., Saccharomyces cerevisiae, Pichia pastoris) transformed with a recombinant yeast expression vector containing a desired peptide encoding nucleotide sequence; insect cell systems infected with a recombinant virus expression vector (e.g., baculovirus) containing a desired peptide encoding nucleotide sequence; plant cell systems infected with a recombinant virus expression vector (e.g., cauliflower mosaic virus, CaMV; tobacco mosaic virus, TMV), or transformed with a recombinant plasmid expression vector (e.g., Ti plasmid), containing a desired peptide encoding nucleotide sequence; or mammalian cell systems (e.g., COS, CHO, BHK, 293, 3T3) harboring a recombinant expression construct containing a desired peptide encoding nucleotide sequence and a promoter derived from the genome of mammalian cells (e.g., metallothionein promoter) or from mammalian viruses (e.g., the adenovirus late promoter, the vaccinia virus 7.5K promoter).

In bacterial systems, a number of different expression vectors may be advantageously selected depending upon the use intended for the desired gene product being expressed. For example, when a large quantity of such a protein is to be produced, such as for the generation of pharmaceutical compositions comprising a desired peptide, or for raising antibodies to the protein, vectors that direct the expression of high levels of fusion protein products that are readily purified may be desirable. Such vectors include, but are not limited to: the E. coli expression vector pUR278 (Ruther and Müller-Hill, EMBO J. 2:1791-1794, 1983), in which a desired peptide encoding sequence may be ligated individually into the vector in frame with the lacZ coding region so that a fusion protein is produced; pIN vectors (Inouye and Inouye, Nucleic Acids Res. 13:3101-3110, 1985, and Van Heeke and Schuster, J. Biol. Chem. 264:5503-5509, 1989); and the like. pGEX vectors (GE Healthcare, Piscataway, N.J.) may also be used to express a desired peptide moiety as a fusion protein with glutathione S-transferase (GST). In general, such fusion proteins are soluble and can easily be purified from lysed cells by adsorption to glutathione-agarose beads, followed by elution in the presence of free glutathione. The pGEX vectors are designed to include thrombin or factor Xa protease cleavage sites so that the cloned desired peptide encoding gene product can be released from the GST moiety.

In an exemplary insect system, Autographa californica nuclear polyhedrosis virus (AcNPV) is used as a vector to express a desired peptide encoding sequence. The virus grows in Spodoptera frugiperda cells. A desired peptide encoding sequence may be cloned individually into a non-essential region (for example the polyhedrin gene) of the virus, and placed under control of an AcNPV promoter (for example the polyhedrin promoter). Successful insertion of a desired peptide encoding sequence will result in inactivation of the polyhedrin gene and production of non-occluded recombinant virus (i.e., virus lacking the proteinaceous coat coded for by the polyhedrin gene). The recombinant viruses are then used to infect Spodoptera frugiperda cells in which the inserted polynucleotide is expressed (see, e.g., Smith et al., J. Virol. 46:584-593, 1983, and U.S. Pat. No. 4,215,051).

In mammalian host cells, a number of viral-based expression systems may be utilized. In cases where an adenovirus is used as an expression vector, a desired peptide encoding nucleotide sequence may be ligated to an adenovirus transcription/translation control complex, e.g., the late promoter and tripartite leader sequence. This chimeric sequence may then be inserted in the adenovirus genome by in vitro or in vivo recombination. Insertion in a non-essential region of the viral genome (e.g., region E1 or E3) will result in a recombinant virus that is viable and capable of expressing desired peptide products in infected hosts (see, e.g., Logan and Shenk, Proc. Natl. Acad. Sci. USA 81:3655-3659, 1984). Specific initiation signals may also be required for efficient translation of inserted desired peptide encoding nucleotide sequences. These signals include the ATG initiation codon and adjacent sequences. In some cases exogenous translational control signals, including, perhaps, the ATG initiation codon, may be provided. Furthermore, the initiation codon should be in phase with the reading frame of the desired peptide encoding coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Nevins, CRC Crit. Rev. Biochem. 19:307-322, 1986).

In yeast, a number of vectors containing constitutive or inducible promoters may be used. For a review, see, e.g., “Current Protocols in Molecular Biology”, supra, Ch. 13, Bitter et al., Meth. Enzymol. 153:516-544, 1987, “DNA Cloning”, Vol. II, Ch. 3 (Glover, ed., IRL Press, Washington, D.C., 1986); Bitter, Meth. Enzymol. 152:673-684, 1987, “The Molecular Biology of the Yeast Saccharomyces: Life Cycle and Inheritance” (Strathern et al., eds., Cold Spring Harbor Press, Cold Spring Harbor, N.Y., 1981), and “The Molecular Biology of the Yeast Saccharomyces: Metabolism and Gene Expression” (Strathern et al., eds., Cold Spring Harbor Press, Cold Spring Harbor, N.Y., 1982).

In plants, a variety of different plant expression vectors can be used, and expression of a desired peptide encoding sequence may be driven by any of a number of promoters. For example, viral promoters such as the 35S RNA or 19S RNA promoters of CaMV (Brisson et al., Nature 310:511-514, 1984), or the coat protein promoter of TMV (Takamatsu et al., EMBO J. 6:307-311, 1987) may be used. Alternatively, plant promoters such as the promoter of the small subunit of RUBISCO (Coruzzi et al., EMBO J. 3:1671-1679, 1984, and Broglie et al., Science 224:838-843, 1984), or heat shock promoters, e.g., soybean hsp17.5-E or hsp17.3-B (Gurley et al., Mol. Cell. Biol. 6:559-565, 1986) may be used. These constructs can be introduced into plant cells using, for example, Ti plasmids, Ri plasmids, plant virus vectors, direct DNA transformation, microinjection, or electroporation. For reviews of such techniques, see, e.g., Weissbach and Weissbach, in “Methods in Plant Molecular Biology”, Section VIII (Schuler and Zielinski, eds., Academic Press, Inc., New York, N.Y., 1988), and “Plant Molecular Biology”, 2^{nd} Ed., Ch. 7-9 (Grierson and Covey, eds., Blackie & Son, Ltd., Glasgow, Scotland, United Kingdom, 1988).

In addition, a host cell strain may be chosen that modulates the expression of the inserted desired peptide encoding sequence, or modifies and processes the desired peptide encoding nucleic acid sequence in a desired fashion. Such modifications (e.g., glycosylation) and processing (e.g., cleavage) of protein products may affect certain functions of the protein. Different host cells have characteristic and specific mechanisms for post-translational processing and modification of proteins and peptides. Appropriate cell lines or host systems can be chosen to ensure the correct or desired modification and processing of the desired protein, polypeptide, or peptide expressed. To this end, eukaryotic host cells that possess the cellular machinery for desired processing of the primary transcript, and glycosylation and/or phosphorylation of desired peptide encoding nucleic acid sequence be used. Such mammalian host cells include, but are not limited to, Chinese hamster ovary (CHO), VERO, baby hamster kidney (BHK), HeLa, monkey kidney (COS), MDCK, 293, 3T3, WI38, human hepatocellular carcinoma (e.g., Hep G2), and U937 cells.

In some embodiments, a recombinant host cell comprising one of the nucleic acid sequences described. In some embodiments, a protein composition comprising one of the polypeptides described.

### II. Methods of Use

In some embodiments, molecular engineered variants (some with improved activity) of the described anion-conducting channelrhodopsin by site-specific mutagenesis and chimera construction. In some embodiments, the channelrhodopsins serve as receptors for phototaxis and the photophobic response. Their photoexcitation initiates depolarization or hyperpolarization of the cell membrane or in the case of neurons with high cytoplasmic chloride concentration, wherein the expressed rhodopsin depolarizes the plasma membrane.

In some embodiments, the rhodopsin domains of several anion-conducting channelrhodopsins were cloned and determined to have channel activity when they were expressed in mammalian HEK293 cells. Using these methods new anion-conducting channelrhodopsin variants, were determined to have improved properties with regards to, among other applications, optogenetics.

One of the major challenges for optogenetic applications, especially in living animals, are scattering of the stimulating light by biological tissues and its absorption by hemoglobin. Optogenetic tools with long-wavelength absorption would exhibit minimal light attenuation from these effects, but most microbial rhodopsins do not fall into this category. For instance, the absorption maximum of ChR2, which possesses several other useful properties and is thereby most frequently used as a depolarizing tool in optogenetics, is 470 nm.

Long-wavelength absorption by optogenetic tools is generally considered desirable to increase the penetration depth of the stimulus light by minimizing tissue scattering and absorption by hemoglobin. In some embodiments, the long-wavelength sensitivity of optogenetic microbial rhodopsins is enhanced using 3,4-Dehydroretinal (A2 retinal). A2 retinal (3,4-dehydroretinal) is a natural retinoid, its 11-cis form being found in photoreceptor cells of certain invertebrates, fish and amphibians, where it may constitute the only retinal, or an additional chromophore to A1 retinal. The presence of an additional double bond in the β-ionone ring of the chromophore results in pigments that absorb light at longer wavelengths, as compared to those formed with A1 (regular) retinal. Variations in A1/A2 ratio cause natural adaptive tuning of spectral sensitivity of vision in the organisms during adaptation to external conditions. Reconstitution of bleached microbial rhodopsins (bacteriorhodopsin, halorhodopsin, sensory rhodopsins I and II) in vitro with all-trans 3,4-dehydroretinal (A2 retinal) also shifts their absorption spectra to longer wavelengths. In some embodiments, spectral properties of optogenetic tools were modified by incorporation of all-trans A2 retinal. The addition of A2 retinal, both ion pumps and channelrhodopsins form functional pigments with significantly red-shifted absorption.

In some embodiments, the long-wavelength sensitivity of optogenetic microbial rhodopsins is enhanced using A2 retinal. In some embodiments, chromophore substitution provides a complementary strategy to improve the efficiency of optogenetic tools. Substitution of A1 retinal by A2 retinal significantly shifts the spectral sensitivity of tested rhodopsins to longer wavelengths typically without altering other aspects of their function.

Optogenetic techniques involve the introduction of light-activated channels and enzymes that allow manipulation of neural activity and control of neuronal function. Thus, in some embodiments, the disclosed methods and compositions can be introduced into cells and facilitate the manipulation of the cells activity and function. See, for example, US publication 20130090454 of U.S. application Ser. No. 13/622,809, as well as, Mattis, J., Tye, K. M., Ferenczi, E. A., Ramakrishnan, C., O'Shea, D. J., Prakash, R., Gunaydin, L. A., Hyun, M., Fenno, L. E., Gradinaru, V., Yizhar, O., and Deisseroth, K. (2012) Principles for applying optogenetic tools derived from direct comparative analysis of microbial opsins. Nat. Methods 9, 159-172; and Zhang, F., Vierock, J., Yizhar, O., Fenno, L. E., Tsunoda, S., Kianianmo-meni, A., Prigge, M., Berndt, A., Cushman, J., Polle, J., Magnuson, J., Hege-mann, P., and Deisseroth, K. (2011) The microbial opsin family of optogenetic tools. Cell 147, 1446-1457).

Optogenetic techniques, and thus the disclosed methods and compositions, can be used to characterize the functions of complex neural circuits and information processing in the normal brain and during various neurological conditions; functionally map the cerebral cortex; characterize and manipulate the process of learning and memory; characterize and manipulate the process of synaptic transmission and plasticity; provide light-controlled induction of gene expression; provide optical control of cell motility and other activities.

Clinical applications of the disclosed methods and compositions include (but are not limited to) optogenetic approaches to therapy such as: restoration of vision by introduction of channelrhodopsins in post-receptor neurons in the retina for ocular disorder gene-therapy treatment of age-dependent macular degeneration, diabetic retinopathy, and retinitis pigmentosa, as well as other conditions which result in loss of photoreceptor cells; control of cardiac function by using channelrhodopsins incorporated into excitable cardiac muscle cells in the atrioventricular bundle (bundle of His) to control heart beat rhythm rather than an electrical pacemaker device; restoration of dopamine-related movement dysfunction in Parkinsonian patients; amelioration of depression; recovery of breathing after spinal cord injury; provide noninvasive control of stem cell differentiation and assess specific contributions of transplanted cells to tissue and network function. Any group of electrically active cells may be amenable to ACR suppression, including, but not limited to those listed above and cardiomyocytes. Such ACRs are also potentially useful for efficient photoinhibition of cardiomyocyte action potentials thereby enabling treatment of cardiac dysfunctions including, but not limited to, tachycardia. In some embodiments, the presently described compositions and methods can be used to facilitate optical stimulation of cardiac cells and tissues, without negative electrophysiological effects of current cardiac anti-arrhythmia therapies and alleviate symptoms by stimulating or silencing specific regions with abnormal excitation in the heart or the brain. In some embodiments, such optogenetic-based techniques could be used to silence, restore or reset irregular heartbeat in patients some of which now receive implantable devices. In some embodiments, ACR based reversible silencing of neuronal activity provides an effective method to isolate specific neuronal populations, thus facilitating modifications in nociceptive pain, behavior, learning and locomotion, for example, when motor neurons are silenced.

In some embodiments, the presently described compositions and methods can be used to influence cardiac cells or regions by either direct viral gene delivery (such as but not limited to AAV) or by delivery of ACR-carrying donor cells (such as but not limited to cardiomyocytes, Purkinje and His bundle cells, etc.) generated or transformed in culture.

In some embodiments, a method of membrane hyperpolarization of a cell in a subject suffering from a neuron mediated disorder, or in the case of neurons with high cytoplasmic chloride concentration, wherein the expressed rhodopsin depolarizes the plasma membrane, said method comprising: delivering to the cell of said subject an expression vector comprising a polynucleotide that encodes an amino acid sequence selected from the group consisting of SEQ ID NO: 1, SEQ ID NO: 3, SEQ ID NO: 5, SEQ ID NO: 7, SEQ ID NO: 9, SEQ ID NO: 47 or SEQ ID NO: 49 (or SEQ ID NO: 56-69 or 105-118), which encodes a rhodopsin domain of an anion-conducting channelrhodopsin expressible in said cell; and expressing said vector in said cell, wherein the expression of the rhodopsin results in membrane hyperpolarization or, for example, in the case of neurons with high cytoplasmic chloride concentration, wherein the expressed rhodopsin depolarizes the plasma membrane.

In some embodiments, a method of neuronal silencing in a subject suffering from a neuron mediated disorder, said method comprising: delivering to a target neuron of said subject an expression vector comprising a polynucleotide that encodes an amino acid sequence selected from the group consisting of SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118, which encodes a rhodopsin domain of an anion-conducting channelrhodopsin expressible in said target neuron; and expressing said vector in said target neuron, wherein the expression of the rhodopsin results in silencing of the signal from the target neuron.

In some embodiments, a method of restoring photosensitivity to a retina of a subject suffering from vision loss or blindness, said method comprising: delivering to the retina of said subject an expression vector comprising a polynucleotide that encodes an amino acid sequence selected from the group consisting of SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118 which encodes a humanized rhodopsin domain of an anion-conducting channelrhodopsin expressible in a retinal neuron; and expressing said vector in said retinal neuron, wherein the expressed rhodopsin renders a high level of membrane potential in said retinal neuron.

Therefore, in some embodiments an anion-conducting channelrhodopsin, light-gated anion channels that provide membrane hyperpolarization is provided and may be used to enhance optogenetic techniques and optogenetic approaches to therapy.

Anion-conducting channelrhodopsins, functional or active portions thereof, such as but not limited to the rhodopsin domain, and functional equivalents include, but are not limited to, naturally occurring versions of ACR and those that are orthologs and homologs, and mutant versions of ACR, whether naturally occurring or engineered (by site directed mutagenesis, gene shuffling, or directed evolution, as described in, for example, U.S. Pat. No. 5,837,458). Also included are the use of degenerate nucleic acid variants (due to the redundancy of the genetic code) of the disclosed algae ACR derived polynucleotide sequences.

In some embodiments, are methods of treating a neuronal disorder, comprising: (a) delivering to a target neuron a nucleic acid expression vector that encodes a rhodopsin domain of an anion-conducting channelrhodopsin, expressible in said target neuron, said vector comprising an open reading frame encoding the rhodopsin domain of an anion-conducting channelrhodopsin, operatively linked to a promoter sequence, and optionally, a transcriptional regulatory sequence; and (b) expressing said vector in said target neuron, wherein the expressed rhodopsin that results in membrane hyperpolarization and neuronal silencing of said target neuron upon exposure to light. In some embodiments, the rhodopsin domain is encoded by SEQ ID NO: 2, SEQ ID NO: 4, SEQ ID NO: 6, SEQ ID NO:8, SEQ ID NO: 10-46, SEQ ID NO: 48, SEQ ID NO: 50-55 or SEQ ID NO: 70-97.

In some embodiments, are methods of treating a neuronal disorder, comprising: (a) delivering to a target neuron a nucleic acid expression vector that encodes a rhodopsin domain of an anion-conducting channelrhodopsin derived from algae, expressible in said target neuron, said vector comprising an open reading frame encoding the rhodopsin domain of an anion-conducting channelrhodopsin, operatively linked to a promoter sequence, and optionally, a transcriptional regulatory sequence; and (b) expressing said vector in said target neuron, wherein the expressed rhodopsin silences said target neuron upon exposure to light. In some embodiments, the rhodopsin domain is encoded by SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118.

In some embodiments, are methods of restoring light sensitivity to a retina, comprising: (a) delivering to a retinal neuron a nucleic acid expression vector that encodes a rhodopsin domain of an anion-conducting channelrhodopsin, expressible in the retinal neuron; said vector comprising an open reading frame encoding the rhodopsin domain of an anion-conducting channelrhodopsin operatively linked to a promoter sequence, and optionally, a transcriptional regulatory sequence; and (b) expressing said vector in said retinal neuron, wherein the expressed rhodopsin renders said retinal neuron photosensitive, thereby restoring light sensitivity to said retina or a portion thereof. In some embodiments, the rhodopsin domain is encoded by SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118.

In some embodiments, are methods of restoring light sensitivity to a retina, comprising: (a) delivering to a retinal neuron a nucleic acid expression vector that encodes a rhodopsin domain of an anion-conducting channelrhodopsin, expressible in the retinal neuron; said vector comprising an open reading frame encoding the rhodopsin domain of an anion-conducting channelrhodopsin operatively linked to a promoter sequence, and optionally, a transcriptional regulatory sequence; and (b) expressing said vector in said retinal neuron, wherein the expressed rhodopsin renders said retinal neuron photosensitive, thereby restoring light sensitivity to said retina or a portion thereof. In some embodiments, the rhodopsin domain is encoded by SEQ ID NO: 2, SEQ ID NO: 4, SEQ ID NO: 6, SEQ ID NO:8, SEQ ID NO: 10-46, SEQ ID NO: 48, SEQ ID NO: 50-55 or SEQ ID NO: 70-97.

In some embodiments, are methods of restoring photosensitivity to a retina of a subject suffering from vision loss or blindness in whom retinal photoreceptor cells are degenerating or have degenerated and died, said method comprising: (a) delivering to the retina of said subject a nucleic acid vector that encodes a rhodopsin domain of an anion-conducting channelrhodopsin expressible in a retinal neuron; said vector comprising an open reading frame encoding the rhodopsin domain of an anion-conducting channelrhodopsin operatively linked to a promoter sequence, and optionally, a transcriptional regulatory sequence; and (b) expressing said vector in said retinal neuron, wherein the expressed rhodopsin renders said retinal neuron photosensitive, thereby restoring photosensitivity to said retina or a portion thereof. In some embodiments, the rhodopsin domain is encoded by SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118.

In some embodiments, the presently described compositions and methods can be used to facilitate optical stimulation of cardiac cells and tissues, without negative electrophysiological effects of current cardiac anti-arrhythmia therapies and alleviate symptoms by stimulating or silencing specific regions with abnormal excitation in the heart or the brain. In some embodiments, such optogenetic based techniques could be used to silence, restore or reset irregular heartbeat in patients some of which now receive implantable devices.

In some embodiments, the presently described compositions and methods can be used to influence cardiac cells or regions by either direct viral gene delivery (such as but not limited to AAV) or by delivery of ACR-carrying donor cells (such as but not limited to cardiomyocytes, Purkinje and His bundle cells, etc.) generated or transformed in culture.

In some embodiments is a method of treating a disorder in an electrically active cell in a subject suffering from a disorder that involves electrically active cells, said method comprising: (a) delivering to the cell of said subject an expression vector comprising a polynucleotide that encodes an amino acid sequence selected from SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118 expressible in said cell; and (b) expressing said vector in said electrically active cell, wherein the expressed rhodopsin silences the signal from said electrically active cell.

In some embodiments is a method of treating a disorder in an electrically active cell in a subject suffering from a disorder that involves electrically active cells, said method comprising: (a) delivering to said subject a transgenic cell comprising an expression vector comprising a polynucleotide that encodes an amino acid sequence selected from SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118 expressible in said transgenic cell; and (b) expressing said vector in said transgenic cell, wherein the expression silences the signal from a electrically active cell.

In some embodiments is a method of silencing an electrically active cell in a subject suffering from an electrically active cell mediated disorder, said method comprising: (a) delivering to a target neuron of said subject an expression vector comprising a polynucleotide that encodes an amino acid sequence selected from SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118 which encodes a rhodopsin domain of an anion-conducting channelrhodopsin expressible in said target neuron; and (b) expressing said vector in said target electrically active cell, wherein the expressed rhodopsin results in silencing of the signal from the electrically active cell. In some embodiments there is provided a recombinant host cell, wherein said host cell is an isolated electrically active cell.

In some embodiments, a method of treating a neuronal disorder comprises: (a) delivering to a target neuron a nucleic acid expression vector that encodes a rhodopsin domain of an anion-conducting channelrhodopsin, expressible in said target neuron; said vector comprising an open reading frame encoding the rhodopsin domain of an anion-conducting channelrhodopsin operatively linked to a promoter sequence, and optionally, transcriptional regulatory sequences; and (b) expressing the expression vector in the target neuron, wherein the expressed anion-conducting channelrhodopsin silences the target neuron upon exposure to light. In some embodiments an above-described expression vector also comprises one or more transcriptional regulatory sequences operably linked to the promoter and rhodopsin domain sequences. In some embodiments, the rhodopsin domain of a anion-conducting channelrhodopsin has the amino acid sequence of all or part of SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118 and the rhodopsin domain sequences of SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118, or a biologically active fragment thereof that retains the biological activity of the encoded rhodopsin domain of a channelrhodopsin or is a biologically active conservative amino acid substitution variant of SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118 or of said fragment. In some embodiments, the expression vector comprises an AAV viral vector. In some embodiments, the promoter is a constitutive promoter. In some embodiments, the constitutive promoter is a CMV promoter or a hybrid CMV enhancer/chicken β-actin (CAG) promoter. In some embodiments, the promoter is an inducible and/or a cell type-specific promoter.

In some embodiments, a method of restoring light sensitivity to a retina comprises (a) delivering to a retinal neuron in a subject a nucleic acid expression vector that encodes a rhodopsin domain of an anion-conducting channelrhodopsin, expressible in the retinal neuron; said expression vector comprising an open reading frame encoding the rhodopsin domain of an anion-conducting channelrhodopsin operatively linked to a promoter sequence, and optionally, one or more transcriptional regulatory sequences; and (b) expressing the expression vector in the retinal neuron, wherein the expressed rhodopsin renders the retinal neuron photosensitive, thereby restoring light sensitivity to the retina or a portion thereof. In some embodiments, the rhodopsin domain of the anion-conducting channelrhodopsin has the amino acid sequence of all or part of SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118, or a biologically active fragment thereof that retains the biological activity of the encoded rhodopsin domain of an anion-conducting channelrhodopsin or is a biologically active conservative amino acid substitution variant of SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118, or of said fragment. In some embodiments, the expression vector comprises an AAV (e.g., AAV2) viral vector. In some embodiments, the promoter is a constitutive promoter. In some embodiments, the constitutive promoter is a CMV promoter or a hybrid CMV enhancer/chicken β-actin (CAG) promoter. In some embodiments, the promoter is an inducible and/or a cell type-specific promoter.

In some embodiments, a method of restoring photosensitivity to a retina of a subject suffering from vision loss or blindness in whom retinal photoreceptor cells are degenerating or have degenerated and died comprises: (a) delivering to the retina of the subject a nucleic acid expression vector that encodes a rhodopsin domain of an anion-conducting channelrhodopsin expressible in retinal neurons; said expression vector comprising an open reading frame encoding the rhodopsin domain of an anion-conducting channelrhodopsin operatively linked to a promoter sequence, and optionally, transcriptional regulatory sequences; and (b) expressing the expression vector in the retinal neuron, wherein the expression of the rhodopsin renders the retinal neuron photosensitive, thereby restoring photosensitivity to said retina or a portion thereof. In some embodiments, the rhodopsin domain of an anion-conducting channelrhodopsin has the amino acid sequence of all or part of SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118 or the rhodopsin domain sequences of SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118, or a biologically active fragment thereof that retains the biological activity of the encoded rhodopsin domain of an anion-conducting channelrhodopsin or is a biologically active conservative amino acid substitution variant SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118, or of said fragment. In some embodiments, the expression vector comprises an AAV (e.g., AAV2) viral vector. In some embodiments, the promoter is a constitutive promoter. In some embodiments, the constitutive promoter is a CMV promoter or a hybrid CMV enhancer/chicken β-actin (CAG) promoter. In other embodiments, the promoter is an inducible and/or a cell type-specific promoter.

A. Compositions as Therapeutics

The use of channelrhodopsins, or active fragments thereof such as but not limited to the rhodopsin domain as therapeutics. In certain embodiments the presently disclosed compositions and are used to improve optogenetic techniques and applications as well as can be used to aid in diagnosis, prevention, and/or treatment of among other things neuron mediated disorders, neurologic disorders (such as Parkinson's disease) and as therapy for ocular disorders.

In certain embodiments the presently disclosed compositions can be administered in combination with one or more additional compounds or agents (“additional active agents”) for the treatment, management, and/or prevention of among other things neuron mediated disorders, neurologic disorders (such as Parkinson's disease) and as therapy for ocular disorders. Such therapies can be administered to a patient at therapeutically effective doses to treat or ameliorate, among other things, neuron mediated disorders, neurologic disorders (such as Parkinson's disease) and as therapy for ocular disorders. A therapeutically effective dose refers to that amount of the compound sufficient to result in any delay in onset, amelioration, or retardation of disease symptoms.

Toxicity and therapeutic efficacy of such compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, expressed as the ratio LD₅₀/ED₅₀. Compositions that exhibit large therapeutic indices are preferred. Compounds that exhibit toxic side effects may be used in certain embodiments, however, care should usually be taken to design delivery systems that target such compositions preferentially to the site of affected tissue, in order to minimize potential damage to uninfected cells and, thereby, reduce side effects.

Data obtained from cell culture assays and animal studies can be used in formulating a range of dosages for use in humans. The dosages of such compositions lie preferably within a range of circulating concentrations that include the ED₅₀ with little or no toxicity. The dosage may vary within this range depending on the dosage form employed and the route of administration utilized. For any composition, the therapeutically effective dose can be estimated initially from cell culture assays. A dose may be formulated in animal models to achieve a circulating plasma concentration range that includes the IC₅₀ (i.e., the concentration of the test composition that achieves a half-maximal inhibition of symptoms) as determined in cell culture. Such information can be used to more accurately determine useful doses in humans. Plasma levels may be measured, for example, by high performance liquid chromatography.

When the therapeutic treatment of among other things neurologic disorders (such as Parkinson's disease) and as therapy for ocular disorders is contemplated, the appropriate dosage may also be determined using animal studies to determine the maximal tolerable dose, or MTD, of a bioactive agent per kilogram weight of the test subject. In general, at least one animal species tested is mammalian. Those skilled in the art regularly extrapolate doses for efficacy and avoiding toxicity to other species, including human. Before human studies of efficacy are undertaken, Phase I clinical studies help establish safe doses.

Additionally, the bioactive agent may be coupled or complexed with a variety of well-established compositions or structures that, for instance, enhance the stability of the bioactive agent, or otherwise enhance its pharmacological properties (e.g., increase in vivo half-life, reduce toxicity, etc.).

Such therapeutic agents can be administered by any number of methods known to those of ordinary skill in the art including, but not limited to, inhalation, subcutaneous (sub-q), intravenous (I.V.), intraperitoneal (I.P.), intramuscular (I.M.), or intrathecal injection, or topically applied (transderm, ointments, creams, salves, eye drops, and the like), as described in greater detail below.

B. Pharmaceutical Compositions

Pharmaceutical compositions for use in accordance with the presently described compositions may be formulated in conventional manners using one or more physiologically acceptable carriers or excipients.

The pharmaceutical compositions can comprise formulation materials for modifying, maintaining, or preserving, for example, the pH, osmolarity, viscosity, clarity, color, isotonicity, odor, sterility, stability, rate of dissolution or release, adsorption or penetration of the composition. Suitable formulation materials include, but are not limited to: amino acids (for example, glycine, glutamine, asparagine, arginine and lysine); antimicrobials; antioxidants (for example, ascorbic acid, sodium sulfite and sodium hydrogen-sulfite); buffers (for example, borate, bicarbonate, Tris-HCl, citrates, phosphates and other organic acids); bulking agents (for example, mannitol and glycine); chelating agents (for example, ethylenediamine tetraacetic acid (EDTA)); complexing agents (for example, caffeine, polyvinylpyrrolidone, beta-cyclodextrin, and hydroxypropyl-beta-cyclodextrin); fillers; monosaccharides, disaccharides, and other carbohydrates (for example, glucose, mannose and dextrins); proteins (for example, serum albumin, gelatin and immunoglobulins); coloring, flavoring, and diluting agents; emulsifying agents; hydrophilic polymers (for example, polyvinylpyrrolidone); low molecular weight polypeptides; salt-forming counterions (for example, sodium); preservatives (for example, benzalkonium chloride, benzoic acid, salicylic acid, thimerosal, phenethyl alcohol, methylparaben, propylparaben, chlorhexidine, sorbic acid and hydrogen peroxide); solvents (for example, glycerin, propylene glycol and polyethylene glycol); sugar alcohols (for example, mannitol and sorbitol); suspending agents; surfactants or wetting agents (for example, pluronics, PEG, sorbitan esters, polysorbates (for example, polysorbate 20 and polysorbate 80), triton, tromethamine, lecithin, cholesterol, and tyloxapal); stability enhancing agents (for example, sucrose and sorbitol); tonicity enhancing agents (for example, alkali metal halides (for example, sodium or potassium chloride), mannitol, and sorbitol); delivery vehicles; diluents; excipients; and pharmaceutical adjuvants (“Remington's Pharmaceutical Sciences”, 18th Ed. (Gennaro, ed., Mack Publishing Company, Easton, Pa., 1990)).

Additionally, the described therapeutic peptides can be linked to a half-life extending vehicle. Certain exemplary half-life extending vehicles are known in the art, and include, but are not limited to, the Fc domain, polyethylene glycol, and dextran (see, e.g., PCT Patent Application Publication No. WO 99/25044).

These agents may be formulated for parenteral administration by injection, e.g., by bolus injection or continuous infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The agents may also be formulated as compositions for rectal administration such as suppositories or retention enemas, e.g., containing conventional suppository bases such as cocoa butter or other glycerides.

In addition to the formulations described previously, the agents may also be formulated as a depot preparation. Such long acting formulations may be administered by implantation (for example subcutaneously or intramuscularly) or by intramuscular injection. For example, agents may be formulated with suitable polymeric or hydrophobic materials (for example as an emulsion in an acceptable oil), ion exchange resins, or as sparingly soluble derivatives, for example, as a sparingly soluble salt. The compositions may, if desired, be presented in a pack or dispenser device, which may contain one or more unit dosage forms containing the active ingredient. The pack may for example comprise metal or plastic foil, such as a blister pack. The pack or dispenser device may be accompanied by instructions for administration.

Active compositions can be administered by controlled release means or by delivery devices that are well-known to those of ordinary skill in the art. Examples include, but are not limited to, those described in U.S. Pat. Nos. 3,845,770, 3,916,899, 3,536,809, 3,598,123, 4,008,719, 5,674,533, 5,059,595, 5,591,767, 5,120,548, 5,073,543, 5,639,476, 5,354,556, and 5,733,566. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof, to provide the desired release profile in varying proportions. Exemplary sustained release matrices include, but are not limited to, polyesters, hydrogels, polylactides (see, e.g., U.S. Pat. No. 3,773,919 and European Patent Application Publication No. EP 058,481), copolymers of L-glutamic acid and gamma ethyl-L-glutamate (see, e.g., Sidman et al., Biopolymers 22:547-556, 1983), poly (2-hydroxyethyl-methacrylate) (see, e.g., Langer et al., J. Biomed. Mater. Res. 15:167-277, 1981, and Langer, Chemtech 12:98-105, 1982), ethylene vinyl acetate (Langer et al., supra), and poly-D(−)-3-hydroxybutyric acid (European Patent Application Publication No. EP 133,988). Sustained release compositions may include liposomes, which can be prepared by any of several methods known in the art (see, e.g., Eppstein et al., Proc. Natl. Acad. Sci. USA 82:3688-3692, 1985, and European Patent Application Publication Nos. EP 036,676, EP 088,046, and EP 143,949). Suitable controlled-release formulations known to those of ordinary skill in the art, including those described herein, can be readily selected for use with the presently disclosed compositions. Certain embodiments encompass single unit dosage forms suitable for oral administration such as, but not limited to, tablets, capsules, gelcaps, and caplets that are adapted for controlled-release.

All controlled-release pharmaceutical products have a common goal of improving therapy over that achieved by their non-controlled counterparts. Ideally, use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of active ingredient that promptly produces the desired therapeutic effect, and gradually and continually release other amounts of active ingredient to maintain this level of therapeutic or prophylactic effect over an extended period of time. In order to maintain this relatively constant level of active ingredient in the body, the drug must be released from the dosage form at a rate that will replace the amount of active ingredient being metabolized and excreted from the body. Controlled-release of an active ingredient can be stimulated by various conditions including, but not limited to, pH, temperature, enzymes, water, or other physiological conditions or compositions.

In some cases, active ingredients of the disclosed methods and compositions are preferably not administered to a patient at the same time or by the same route of administration. Therefore, in some embodiments are kits that, when used by the medical practitioner, can simplify the administration of appropriate amounts of active ingredients to a patient.

A typical kit comprises a single unit dosage form of one or more of the therapeutic agents disclosed, alone or in combination with a single unit dosage form of another agent that may be used in combination with the disclosed compositions. Disclosed kits can further comprise devices that are used to administer the active ingredients. Examples of such devices include, but are not limited to, syringes, drip bags, patches, and inhalers.

Disclosed kits can further comprise pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate. However, in specific embodiments, the disclosed formulations do not contain any alcohols or other co-solvents, oils or proteins.

C. Transgenic Animals

The present disclosure provides methods and compositions for the creation and use of both human and non-human transgenic animals that carry an algae derived anion-conducting channelrhodopsin transgene in all their cells, as well as non-human transgenic animals that carry an algae derived anion-conducting channelrhodopsin transgene in some, but not all their cells, for example in certain electrically active cells. Human and non-human mammals of any species, including, but not limited to, mice, rats, rabbits, guinea pigs, pigs, micro-pigs, goats, and non-human primates, e.g., baboons, monkeys, and chimpanzees, can be used to generate transgenic animals carrying an algae derived anion-conducting channelrhodopsin polynucleotide (and/or expressing an algae derived polypeptide) may be integrated as a single transgene or in concatamers, e.g., head-to-head or head-to-tail tandems. An algae derived anion-conducting channelrhodopsin transgene may also be selectively introduced into and activated in a particular cell-type (see, e.g., Lakso et al., Proc. Natl. Acad. Sci. USA 89:6232-6236, 1992). The regulatory sequences required for such a cell-type specific activation will depend upon the particular cell-type of interest, and will be apparent to those of skill in the art.

Should it be desired that an algae-derived anion-conducting channelrhodopsin, or fragment thereof, transgene be integrated into the chromosomal site of the endogenous copy of the mammalian anion-conducting channelrhodopsin gene, gene targeting is generally preferred. Briefly, when such a technique is to be utilized, vectors containing some nucleotide sequences homologous to the endogenous anion-conducting channelrhodopsin gene are designed for the purpose of integrating, via homologous recombination with chromosomal sequences, into and disrupting the function of the endogenous channelrhodopsin gene (i.e., “knock-out” animals). In this way, the expression of the endogenous channelrhodopsin gene may also be eliminated by inserting non-functional sequences into the endogenous channelrhodopsin gene. The transgene may also be selectively introduced into a particular cell-type, thus inactivating the endogenous channelrhodopsin gene in only that cell-type (see, e.g., Gu et al., Science 265:103-106, 1994). The regulatory sequences required for such a cell-type specific inactivation will depend upon the particular cell-type of interest, and will be apparent to those of skill in the art.

Any technique known in the art may be used to introduce a channelrhodopsin, or fragment thereof, transgene into animals to produce the founder lines of transgenic animals. Such techniques include, but are not limited to: pronuclear microinjection (U.S. Pat. No. 4,873,191); retrovirus-mediated gene transfer into germ lines (van der Putten et al., Proc. Natl. Acad. Sci. USA 82:6148-6152, 1985); gene targeting in embryonic stem cells (Thompson et al., Cell 56:313-321, 1989); electroporation of embryos (Lo, Mol. Cell. Biol. 3:1803-1814, 1983); sperm-mediated gene transfer (Lavitrano et al., Cell 57:717-723, 1989); and positive-negative selection, as described in U.S. Pat. No. 5,464,764. For a review of such techniques, see, e.g., Gordon, Int. Rev. Cytol. 115:171-229, 1989.

Once transgenic animals have been generated, the expression of the recombinant channelrhodopsin gene, or fragment thereof, may be assayed utilizing standard techniques. Initial screening may be accomplished by Southern blot analysis or PCR techniques to analyze animal tissues to assay whether integration of the channelrhodopsin transgene has taken place. The level of mRNA expression of the channelrhodopsin transgene in the tissues of the transgenic animals may also be assessed using techniques that include, but are not limited to, Northern blot analysis of cell-type samples obtained from the animal, in situ hybridization analysis, and RT-PCR. Samples of an algae derived channelrhodopsin-expressing tissue can also be evaluated immunocytochemically using antibodies selective for the channelrhodopsin transgene product.

D. Transgene Based Therapies

In certain embodiments the presently disclosed compositions and are used to improve optogenetic techniques and applications as well as can be used to aid in diagnosis, prevention, and/or treatment of neurologic disorders, such as but not limited to Parkinson's disease, as well as for ocular disorders.

In some embodiments, methods and compositions are used to identify and characterize multiple channelrhodopsins derived from algae. The cloning and expression of the rhodopsin domain of the channelrhodopsins and expression in mammalian cells demonstrates that these channelrhodopsins have improved characteristics that can be used for optogenetic applications as well as therapeutic agents.

For example, a disclosed method and composition may be used in, among other things, retinal gene therapy for mammals (as described in, among others, U.S. Pat. Nos. 5,827,702, 7,824,869 and US Patent Publication Number 20100015095 as well as in WIPO publications WO 2000/15822 and WO 1998/48097). A genetically engineered ocular cell is produced by contacting the cell with an exogenous nucleic acid under conditions in which the exogenous nucleic acid is taken up by the cell for expression. The exogenous nucleic acid is described as a retrovirus, an adenovirus, an adeno-associated virus or a plasmid. Retinal gene transfer of a reporter gene, green fluorescent protein (GFP), using a recombinant adeno-associated virus (rAAV) was demonstrated in normal primates (Bennett, J et al. 1999 Proc. Natl. Acad. Sci. USA 96, 9920-25). The rescue of photoreceptors using gene therapy in a model of rapid degeneration of photoreceptors using mutations of the RP65 gene and replacement therapy with the normal gene to replace or supplant the mutant gene (See, for example, US Patent Publication 2004/0022766) has been used to treat a naturally-occurring dog model of severe disease of retinal degenerations—the RPE65 mutant dog, which is analogous to human LCA. By expressing photosensitive membrane-channels or molecules in surviving retinal neurons of the diseased retina by viral based gene therapy method, the present invention may produce permanent treatment of the vision loss or blindness with high spatial and temporal resolution for the restored vision.

The nucleic acids sequences that encode an active portion of the presently disclosed anion-conducting channelrhodopsins, include but are not limited to the nucleic acid sequences that encode the rhodopsin domains identified in SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118 or the optimized rhodopsin domain sequences of SEQ ID NO: 2, SEQ ID NO: 4, SEQ ID NO: 6, SEQ ID NO:8, SEQ ID NO: 10-46, SEQ ID NO: 48, SEQ ID NO: 50-55 or SEQ ID NO: 70-97.

In some embodiments, introduction and expression of channelrhodopsins, such as those described herein, in ocular neuronal cells, for example, impart light sensitivity to such retinas and restoring one or more aspects of visual responses and functional vision to a subject suffering from such degeneration. By restoring light sensitivity to a retina lacking this capacity, due to disease, a mechanism for the most basic light-responses that are required for vision is provided. In some embodiments, the functional domains of anion-conducting channelrhodopsins, such as GtACR1 and GtACR2 may be used to restore light sensitivity to the retinas that have undergone rod and cone degeneration by expressing the channelrhodopsin in inner retinal neurons in vivo. In some embodiments these channelrhodopsins may be introduced using techniques that include, but are not limited to, retinal implants, cortical implants, lateral geniculate nucleus implants, or optic nerve implants

In some embodiments, the anion-conducting channelrhodopsins are inserted into the retinal neurons that survived after the rods and cones have died in an area or portion of the retina of a subject, using the transfer of nucleic acids, alone or within an expression vector. Such expression vectors may be constructed, for example, by introduction of the desired nucleic acid sequence into a virus system known to be of use for gene therapy applications, such as, but not limited to, AAV (e.g., AAV2), retroviruses and alike.

In some embodiments the anion-conducting channelrhodopsins may be inserted into retinal interneurons. These cells then can become light sensitive and send signals via the optic nerve and higher order visual pathways to the visual cortex where visual perception occurs, as has been demonstrated electrophysiologicly in mice. In some embodiments, among other routes, intravitreal and/or subretinal injections may be used to deliver channelrhodopsin molecules or virus vectors expressing the same.

In some embodiments, the active portion of the presently disclosed algal derived anion-conducting channelrhodopsins, such as but not limited to the rhodopsin domain of these anion-conducting channelrhodopsins, can be used to restore light sensitivity to a retina, by delivering to retinal neurons a nucleic acid expression vector that encodes algal derived anion-conducting channelrhodopsins (such as but not limited to the rhodopsin domain of these anion-conducting channelrhodopsins) that is expressible in the neurons, which vector comprises an open reading frame encoding the rhodopsin, and operatively linked thereto, a promoter sequence, and optionally, transcriptional regulatory sequences; and expressing the vector in the neurons, thereby restoring light sensitivity.

In certain embodiments the channel rhodopsin can be algal derived anion-conducting channelrhodopsins such as, but not limited to functional domains of anion-conducting channelrhodopsins, such as, but not limited to, SEQ ID NO: 1, SEQ ID NO: 3, SEQ ID NO: 5, SEQ ID NO: 7, SEQ ID NO: 9, SEQ ID NO: 47, SEQ ID NO: 49, SEQ ID NO: 56-69 or SEQ ID NO: 105-118 or a biologically active fragment or conservative amino acid substitution variant thereof, such as but not limited to the rhodopsin domain. The vector system may be recombinant AAV (e.g., AAV2), the promoter may be a constitutive promoter such as, but not limited to, a CMV promoter or a hybrid CMV enhancer/chicken β-actin promoter (CAG).

### III. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Materials and Methods

Molecular Biology Methods:

DNA polynucleotides encoding the 7TM domains of transcripts showing homology to G. theta ACRs (described in at least PCT/US2016/023095) that were optimized for human codon usage were synthesized (Genewiz, South Plainfield, N.J.) and cloned into the mammalian expression vector pcDNA3.1 (Life Technologies, Grand Island, N.Y.) in frame with an EYFP tag for expression in HEK293 cells. The sequence information was deposited in GenBank (for accession numbers see Table 1). For expression in neurons, EGFP fusion constructs were transferred to the pFUGW vector backbone (as described in Lois, C., Hong, E. J., Pease, S., Brown, E. J. & Baltimore, D. Germline transmission and tissue-specific expression of transgenes delivered by lentiviral vectors. Science 295, 868-872, 2002). Mutants were generated using Quikchange XL kit (Agilent Technologies, Santa Clara, Calif.) and verified by sequencing.

HEK293 Recording Methods:

HEK293 (human embryonic kidney) cells were transfected using the ScreenFectA transfection reagent (Waco Chemicals USA, Richmond, Va.). All-trans-retinal (Sigma) was added as a stock solution in ethanol at the final concentration of 5 μM. Measurements were performed 48-72 h after transfection with an Axopatch 200B amplifier (Molecular Devices, Union City, Calif.). The signals were digitized with a Digidata 1440A using pClamp 10 software (both from Molecular Devices). Patch pipettes with resistances of 2-5 MΩ were fabricated from borosilicate glass. The pipette solution contained (in mM): KCl 126, MgCl2 2, CaCl2 0.5, Na-EGTA 5, HEPES 25, pH 7.4. The standard bath solution contained (in mM): NaCl 150, CaCl2 1.8, MgCl2 1, glucose 5, HEPES 10, pH 7.4. A 4 M salt bridge was used in all experiments. All current-voltage dependencies were corrected for liquid junction potentials calculated using the ClampEx built-in LJP calculator. Continuous light pulses were provided by a Polychrome IV light source (T.I.L.L. Photonics GMBH, Grafelfing, Germany) in combination with a mechanical shutter (Uniblitz Model LS6, Vincent Associates, Rochester, N.Y., USA; half-opening time 0.5 ms). The light intensity was attenuated with the built-in Polychrome system or with neutral density filters. Maximal quantum density at the focal plane of the 40× objective lens was 7.7 mW/mm2. All measurements were carried out at room temperature (25° C.).

Neuronal Lentiviral Transduction and Recording Methods:

To express PsuACR1_973-EGFP fusion construct in hippocampal neurons, lentivirus was produced in HEK293FT cells (Invitrogen) through triple-transfection of plasmids pCMV-VSVG, pA8.9 and PsuACR1_973-EGFP. The virus was purified from the cell culture supernatant by ultracentrifugation through a 20% Sucrose layer at 20,000 rpm in an SW32 rotor for 2 hours. Neurons isolated from E18 mouse hippocampi (BrainBits, Springfield, Ill., USA) by papain digestion were cultured on top of a glia feeder layer in NbActiv4 medium (BrainBits, Springfield, Ill., USA) supplemented with all-trans retinal (0.4 μM). Neurons were infected with lentivirus one day after plating. Patch clamp measurements were carried out 9-14 days after lentivirus infection. The same photoexcitation source and measuring setup was used as described above for HEK cells, except that neurons were bathed in Tyrode's solution (in mM: KCl 2, NaCl 125, MgCl2 1, CaCl2 3, glucose 30, HEPES 25, pH 7.3) and the pipette solution contained (in mM): K gluconate 135, MgCl2 2, HEPES 20, pH 7.2). Spiking was measured in the current clamp mode at room temperature (25° C.).

Analysis of Neuronal Morphology:

Hippocampal neurons at day 14 post-transduction were fixed in 4% paraformaldehyde in PBS, washed, permeabilized (0.3% Triton X-100 in PBS), washed once more, and blocked with 2% goat serum in PBS for 30 min. They were incubated with rabbit anti-GFP IgG (Life technologies, 1:200) and mouse anti-MAP2 IgG1 (Synaptic Systems, 1:500); or with polyclonal rabbit anti-synapsin antibody (a gift of Dr. Thomas Südhof, The University of Texas Southwestern Medical School, 1:1000) and guinea pig anti-Tau IgG2a (Synaptic Systems, 1:500) at 4° C. overnight. They were washed and secondarily stained with Alexa Fluor 488-conjugated goat anti-rabbit IgG and Alexa Fluor 568-conjugated goat anti-mouse IgG or Alexa Fluor 647-conjugated goat anti-guinea pig IgG at room temperature for 1 hour, washed and mounted with ProLongDiamondAntifade with DAPI mounting medium (Molecular Probes). Images were obtained through the Zen software for the laser scanning microscope (LSM 510 META, Carl Zeiss, Germany). Primary dendrites from randomly selected neurons were identified as MAP2-positive branches originating from the soma and crossing the circumference of a 25 μm radius. Synapses (identified by synapsin staining, as described in Hosaka, M., Hammer, R. E. & Südhof, T. C. A phospho-switch controls the dynamic association of synapsins with synaptic vesicles. Neuron 24, 377-387, 1999) were quantified using PunctaAnalyzer plugin for Image J in a circular area with a radius of 25 μm from the soma as described (Ippolito, D. M. & Eroglu, C. Quantifying synapses: an immunocytochemistry-based assay to quantify synapse number. J. Vis. Exp., November 16; (45), 2270, 2010).

Statistical Methods:

Statistical data are shown as mean±sem. The n values indicate the number of independent experiments (different cells, unless otherwise stated). For analysis of significance unpaired Student's t-test was used, and p values less than 0.05 were considered significant.

### Example 2—Characterization of ACRs

Sequences of the New ACRs.

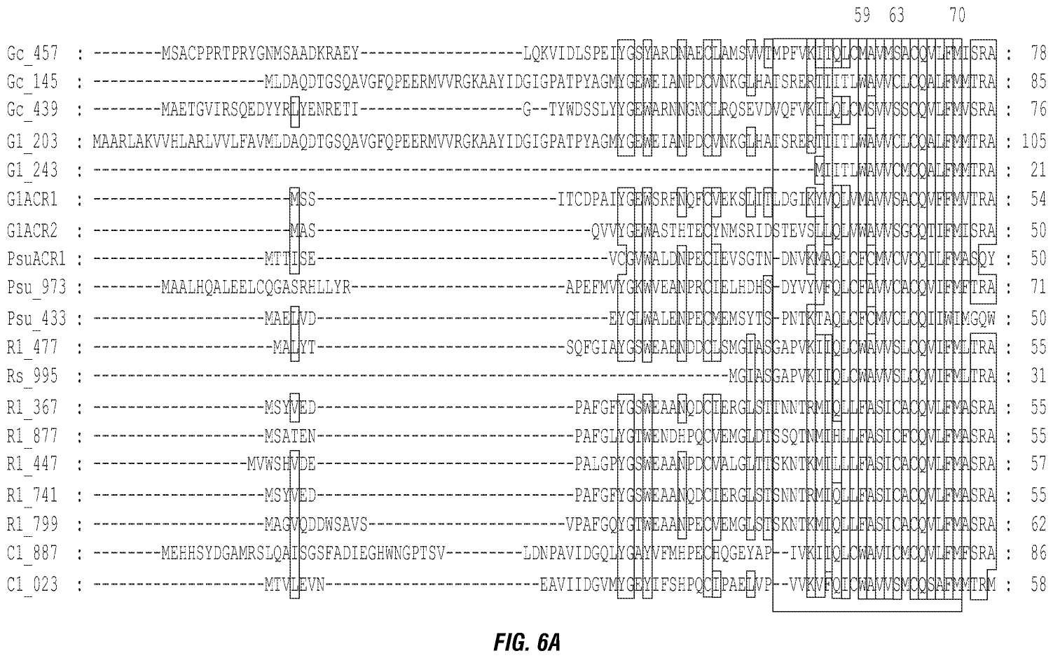

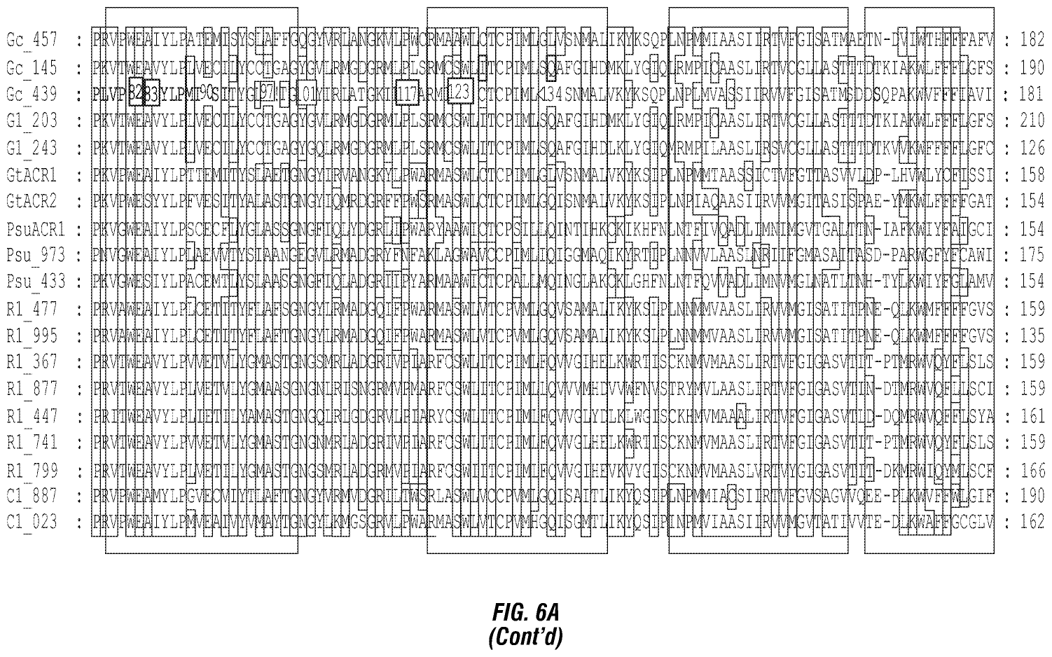

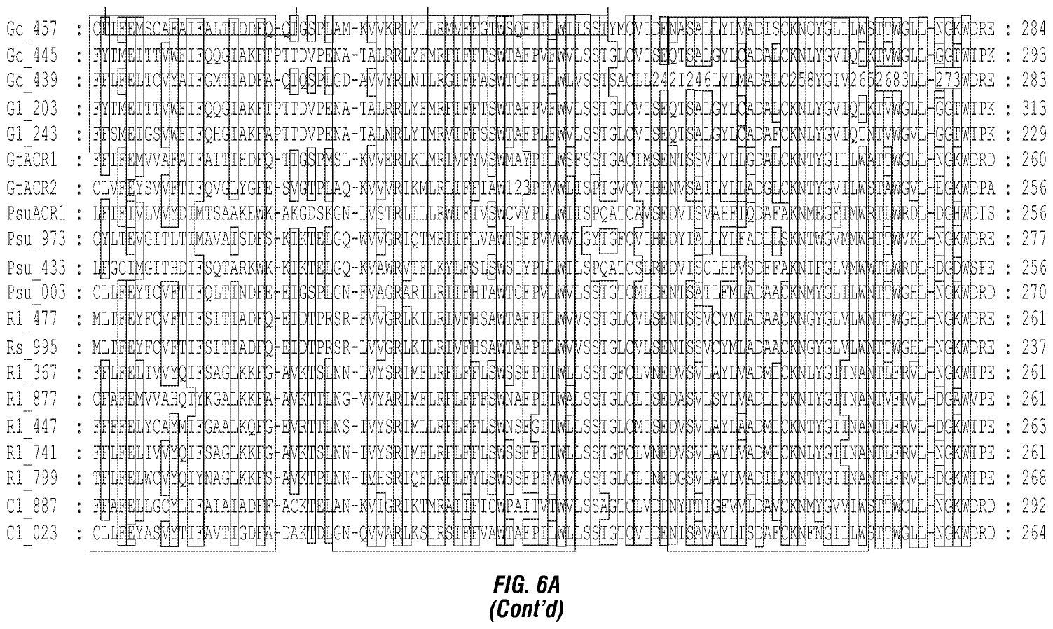

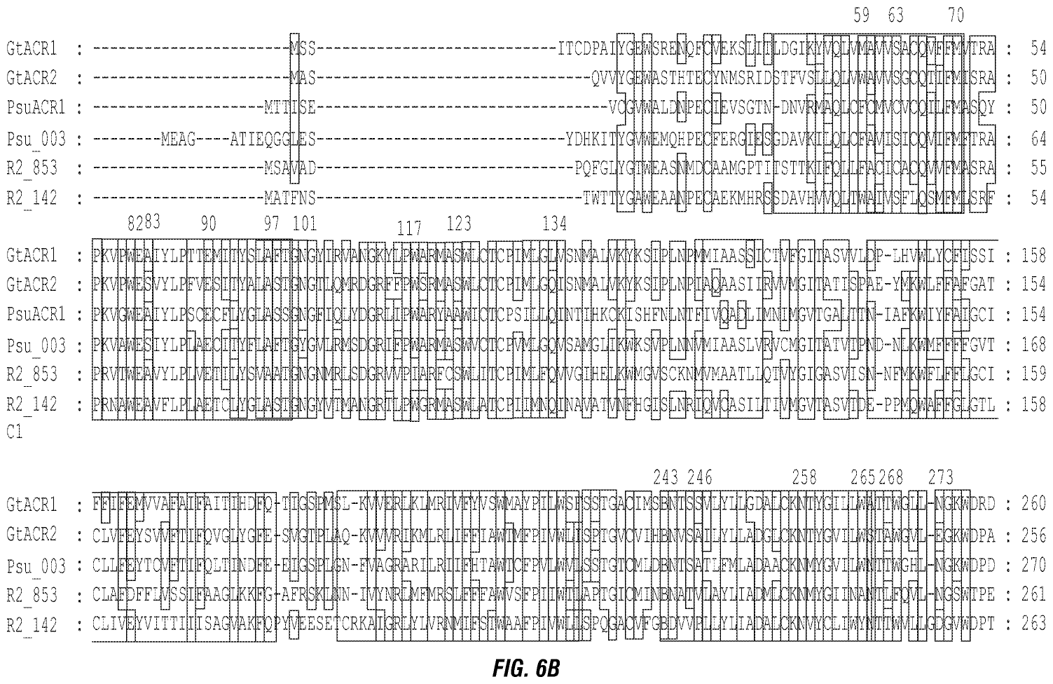

Transcripts were identified that encode proteins homologous to earlier identified ACRs from G. theta and P. sulcata (as described in PCT/US2016/023095, as well as in Govorunova, E. G., Sineshchekov, O. A., Liu, X., Janz, R. & Spudich, J. L. Natural light-gated anion channels: A family of microbial rhodopsins for advanced optogenetics. Science 349, 647-650, 2015 and Govorunova, E. G., Sineshchekov, O. A. & Spudich, J. L. Proteomonas sulcata ACR1: A fast anion channelrhodopsin. Photochem. Photobiol. 92, 257-263, 2016) by searching cryptophyte transcriptomes. Mammalian codon-adapted versions of the seven-transmembrane-helix domains of these homologs were synthesized. Their amino acid alignment is shown in FIGS. 6A and 6B.

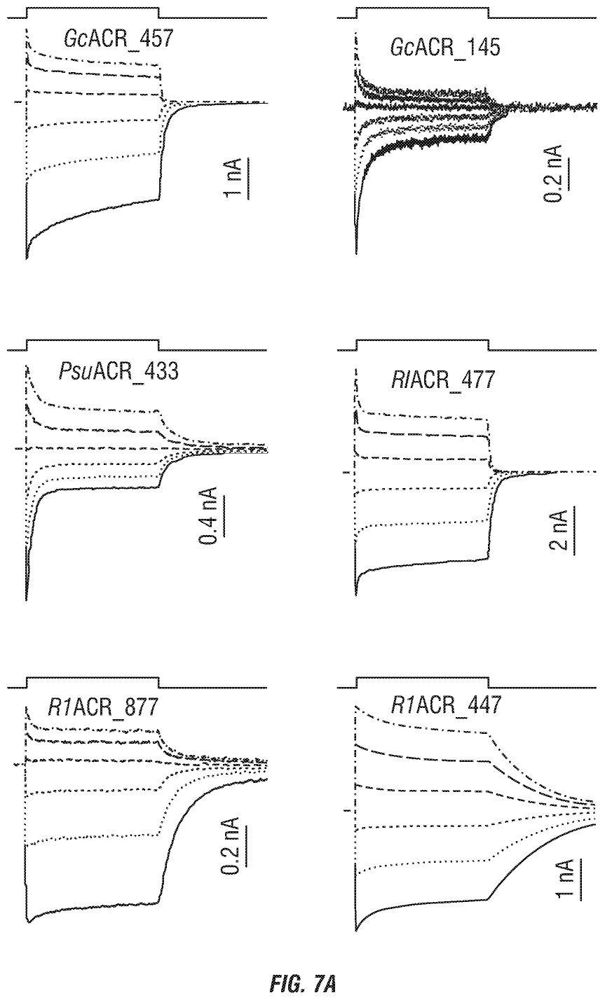

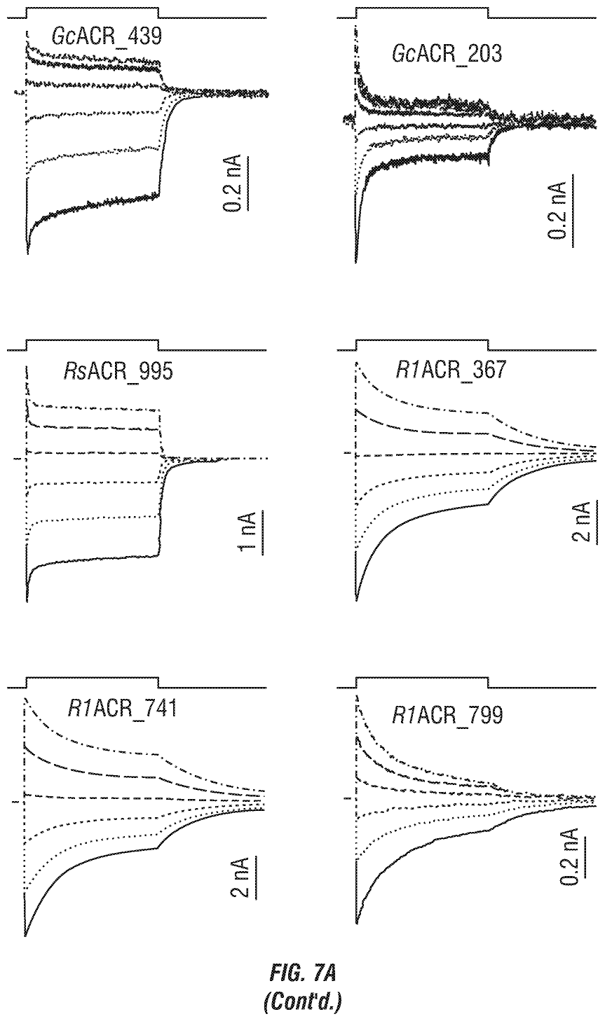

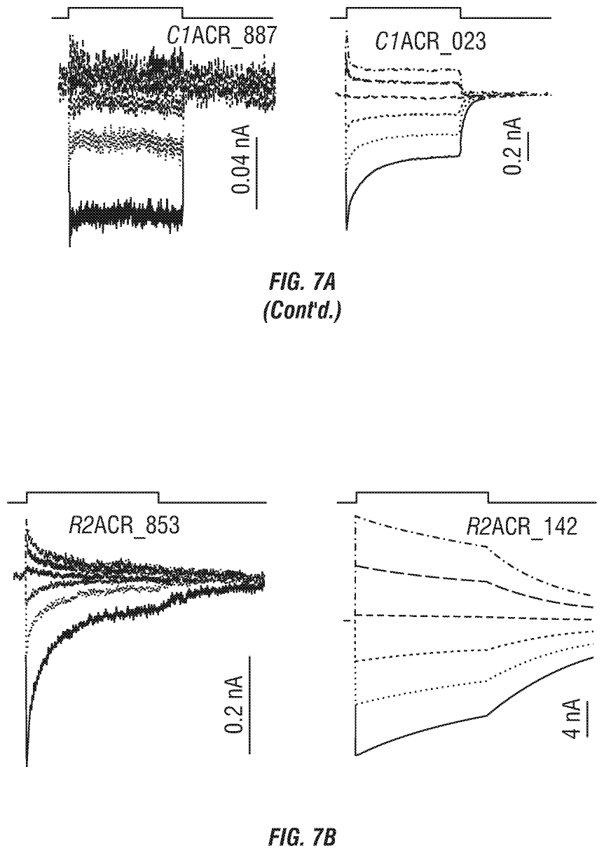

Synthesized transcripts were fused in frame with the C-terminal enhanced yellow fluorescent protein (EYFP) tag, and HEK293 (human embryonic kidney) cells were transfected with the resultant constructs. Seventeen constructs generated transmembrane currents in response to illumination (FIG. 7); two constructs that did not generate currents were excluded from further analysis. The spectral sensitivity was determined as described previously (Govorunova, E. G., Spudich, E. N., Lane, C. E., Sineshchekov, 0. A. & Spudich, J. L. New channelrhodopsin with a red-shifted spectrum and rapid kinetics from Mesostigma viride. mBio 2, e00115-00111, 2011).

GenBank accession numbers, organism sources, transcript names, the wavelength of the maximal sensitivity and protein name abbreviations are listed in Table 1, below.

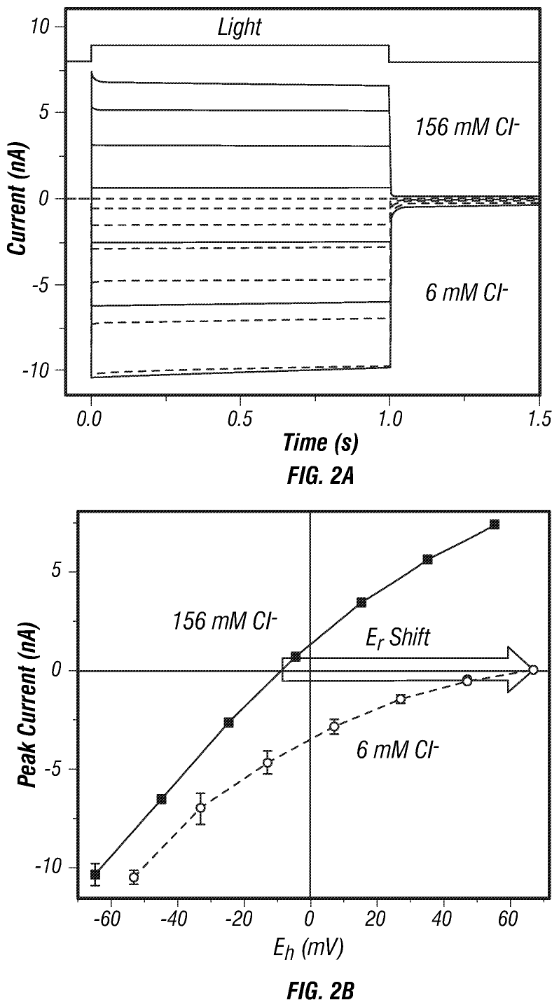

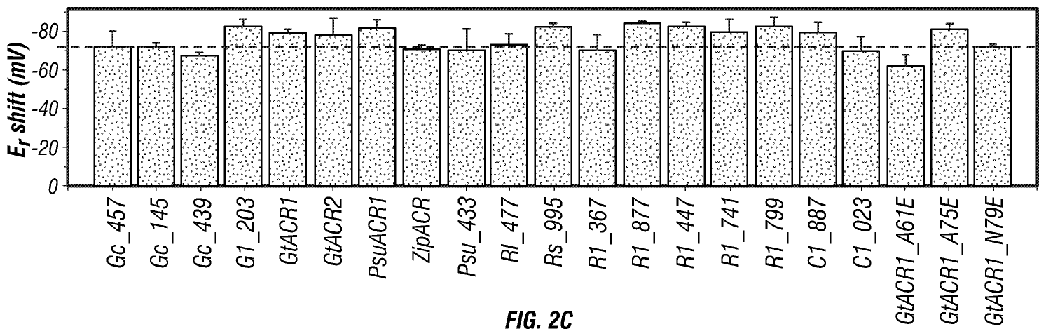

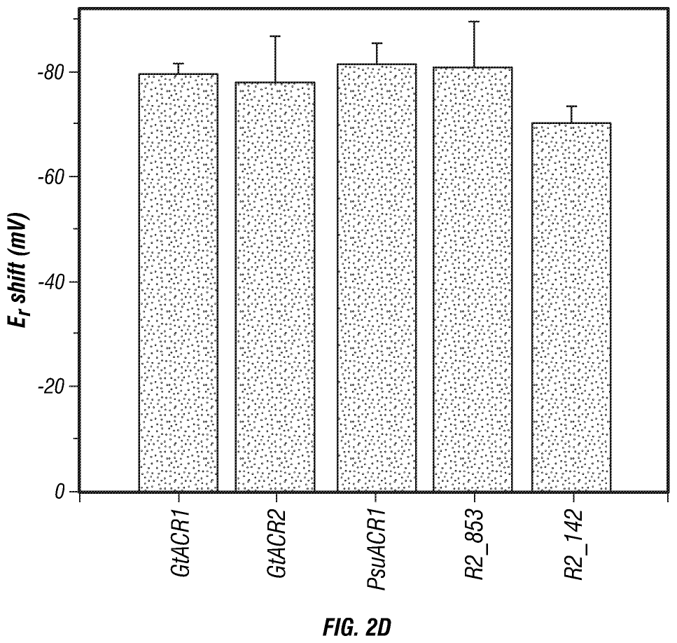

All of the new ACR homologs are natural chloride-conducting channelrhodopsins. To demonstrate that these newly identified ACR homologs indeed conduct anions, the current-voltage relationships and determined the shift of the reversal potentials (Er) upon partial replacement of Cl⁻ in the bath with non-permeable Asp⁻, as was done previously with GtACRs and PsuACR1 (as described in PCT/US2016/023095, incorporated herein by reference). Representative series of currents traces recorded from PsuACR_973 (nicknamed ZipACR based on its robust currents, rapid kinetics, and function in neuron silencing shown below) in the standard bath (black lines) and after replacement (red lines) are shown in FIG. 2A, and the corresponding current-voltage relationships (IE curves) are shown in FIG. 2B. The Er shifts measured in the same manner for all new ACR homologs confirmed that all tested homologs conduct anions (FIGS. 2C and 2D).

Structural Determinants of Anion Selectivity in Natural Channelrhodopsins:

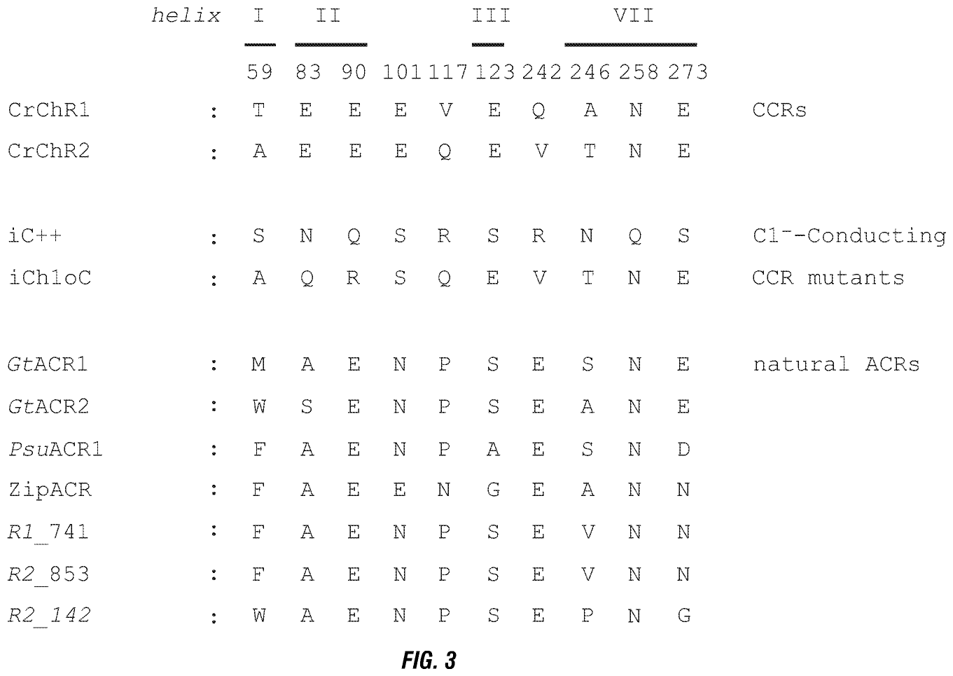

Using a set of 20 confirmed ACRs a protein sequence analysis was performed to compare natural cryptophyte ACRs with Cl−-conducting mutants engineered from chlorophyte CCRs. The first two lines in FIG. 3 show amino acid residues of CCR sequences. The third and fourth lines are the mutations introduced to confer Cl⁻ selectivity to the latest versions of Cl⁻-conductive mutants of CCRs, respectively, iC++ (described in Berndt, A. et al. Structural foundations of optogenetics: Determinants of channelrhodopsin ion selectivity. Proc. Natl. Acad. Sci. USA 113, 822-829, 2015, incorporated herein by reference) and iChloC (Wietek, J. et al. An improved chloride-conducting channelrhodopsin for light-induced inhibition of neuronal activity in vivo. Sci. Rep. 5, 14807, 2015, incorporated herein by reference). The remaining lines show the corresponding residues in six of the confirmed natural ACRs (for the residues in the remaining sequences see FIGS. 6A and 6B). The most striking difference between the artificial and natural Cl−-conductive proteins is the position corresponding to Glu90 in CrChR2: it needed to be replaced with a neutral or positively charged residue to convert a CCR into a Cl⁻ channel, whereas it is present in all natural ACRs. Moreover, replacement of the Glu90 homolog Glu68 in GtACR1 with a neutral (Gln) or alkaline (Lys) residue does not alter its anion selectivity, but rather strongly influences the ACR's gating properties (Sineshchekov, O. A., Govorunova, E. G., Li, H. & Spudich, J. L. Gating mechanisms of a natural anion channelrhodopsin. Proc. Natl. Acad. Sci. USA 112, 14236-14241 (2015), incorporated herein by reference), both of these effects in stark contrast to the corresponding mutations reported in CrChR2 (Wietek, J. et al. Conversion of channelrhodopsin into a light-gated chloride channel. Science 344, 409-412, 2014).

Furthermore, out of two positions at which positive charges were introduced in iC++, one (Gln-117 in CrChR2) is occupied with a neutral residue, and another (Val242 in CrChR2), with a negative charge in all confirmed natural ACRs (FIG. 3 and FIGS. 6A and B). These findings indicate that the anion conduction pathways in natural and artificial ACRs are very different and the conserved Glu90 residue in helix 2 have diverged to serve different roles in the CCR and ACR mechanisms.

In addition to the aforementioned Glu90, most CCRs contain four other Glu residues in the 2nd helix and the loop between helices 2 and 3 (Glu82, Glu83, Glu90, Glu97 and Glu101 in CrChR2). It has been proposed that the absence of Glu residues in the positions corresponding to Glu83, Glu97 and Glu101 contribute to the formation of an anion-conducting pore in natural ACRs 5. Indeed, in nearly all confirmed natural ACRs non-carboxylic residues are found in these three positions (FIG. 6). However in the highly conductive PsuACR_973 (FIG. 2), Glu101 is conserved (FIG. 6), which demonstrates that the presence of a non-carboxylate residue at least in this position is not absolutely necessary for anion conductance.

To further test the hypothesis that the absence of Glu83, Glu97 and Glu101 determines anion conductance of natural ACRs, these glutamates were introduced in the corresponding positions in GtACR1. Glu83 and Glu97 of CrChR2 are located in the 2nd transmembrane helix, which was confirmed by the high-resolution crystal structure of a hybrid channelrhodopsin 17. The corresponding residues in the predicted 2nd helix of GtACR1 are Ala61 and Ala75. Glu101 of CrChR2 is located in the 2nd loop; therefore to determine its counterpart in GtACR1 is more difficult. Gln79 in GtACR1 is separated by 3 residues from Ala75, as is Glu101 from Glu97 in CrChR2. We generated the A61E, A75E and N79E mutants of GtACR1, expressed them in HEK293 cells and measured their IE curves in the standard buffer and upon partial replacement of Cl− with Asp− in the bath, as we did for natural ACR homologs. The shifts of the Er upon such replacement determined for each of these mutants was almost as large as that of natural ACRs (FIGS. 2C and D), although the A61E and A71E mutations strongly decreased the current amplitude (Table 3) Therefore, we conclude that the presence of individual conserved residues in the 2nd helix is not sufficient to predict anion or cation selectivity of channelrhodopsins. This conclusion is corroborated by an earlier observation that Mesostigma viride channelrhodopsin 1 (MvChR1) in which Glu83 and Glu97 homologs are also replaced with neutral residues as in ACRs is in fact a CCR with one of the highest Na+/H+ permeability ratios among CCRs 11.

Functional Diversity in the ACR Family.

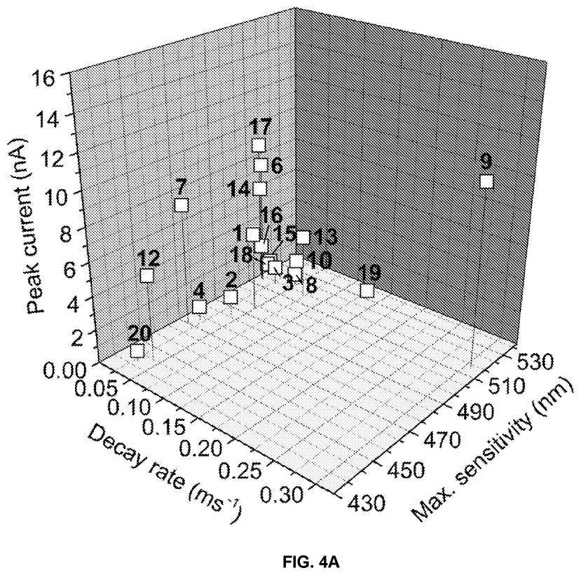

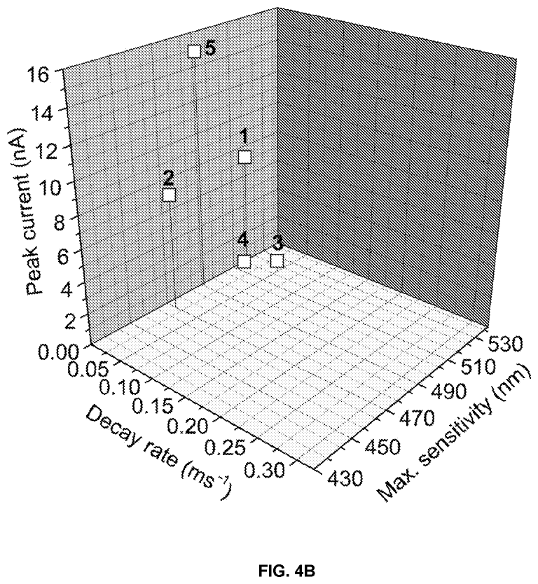

Photocurrents generated by new ACRs greatly differed in amplitude, kinetics and spectral sensitivity, demonstrating diversity of this protein family (FIGS. 4A and 4B and FIGS. 7A and 7B). PsuACR_973, R1ACR_741 and R2ACR_142 generated currents larger than those produced by the previously known ACRs. PsuACR_973 also exhibited the by far fastest current decay among all so far identified ACRs (2-4 ms current half-decay time depending on voltage), the main reason we nicknamed it “ZipACR”. The range of the wavelengths of the ACR spectral maxima extended from 440 (C1ACR_023) to 535 (C1ACR_887) nm.

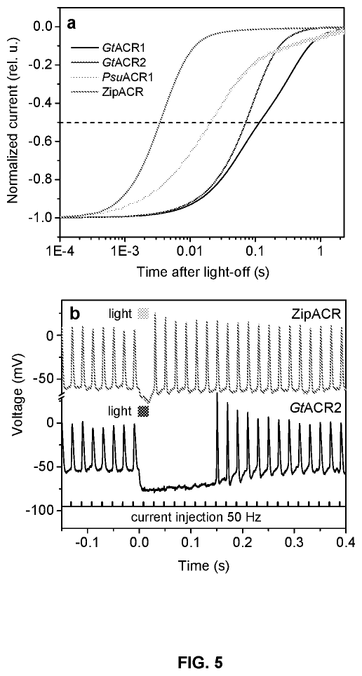

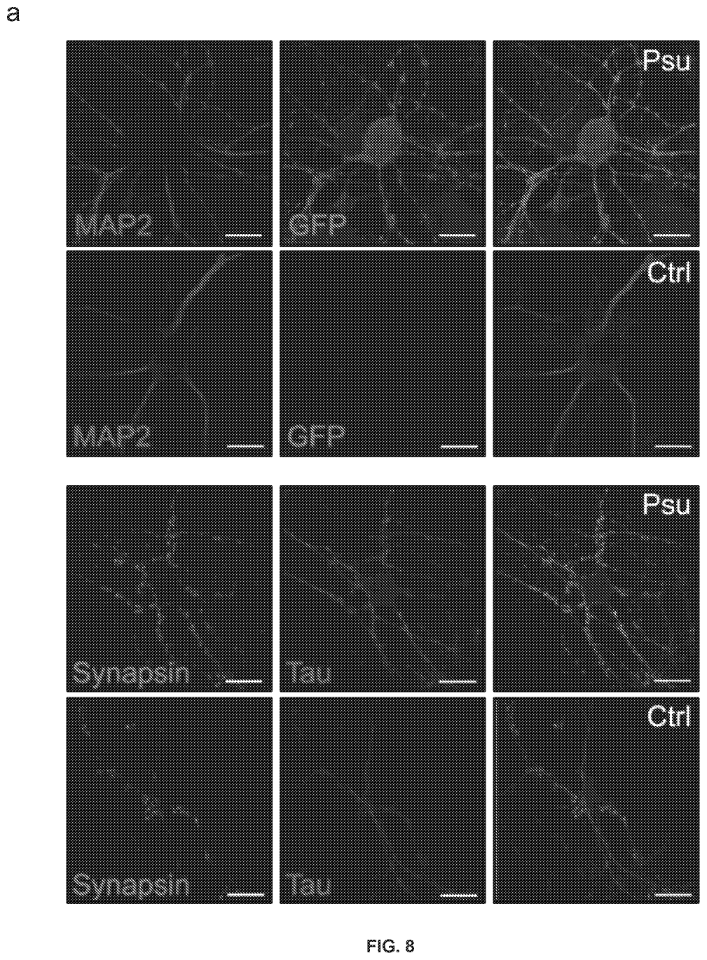

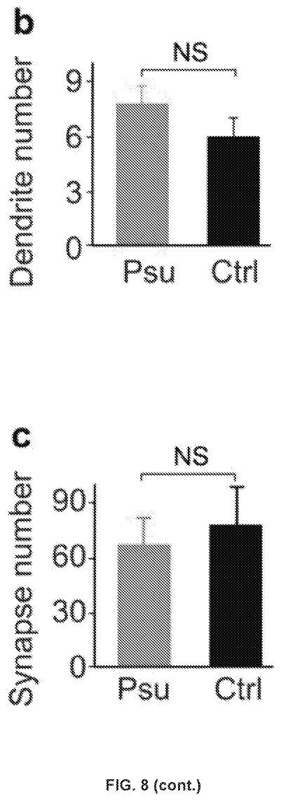

ZipACR allows optical inhibition of individual neuronal spikes at unprecedentally high frequencies. GtACR2, the fastest of the two G. theta ACRs, could be used in neurons to suppress individual spikes at firing frequencies up to 25 Hz (Govorunova, E. G., Sineshchekov, O. A., Liu, X., Janz, R. & Spudich, J. L. Natural light-gated anion channels: A family of microbial rhodopsins for advanced optogenetics. Science 349, 647-650, 2015). However, its current decay is too slow for selective deletion of individual spikes in trains of higher frequency that may be required in certain optogenetic experiments 18. Photocurrents from ZipACR exhibited ˜21 times faster decay than those from GtACR2 in HEK293 cells, their amplitude was ˜40% larger, and the wavelength of the maximal sensitivity was 45 nm red-shifted from that of GtACR2 (FIG. 5A and Table 3). ZipACR were expressed in cultured rat hippocampal neurons under the ubiquitin promoter using lentiviral delivery and tested its performance as an inhibitory optogenetic tool compared to that of GtACR2. Robust expression of ZipACR in neurons was evident from the tag fluorescence, whereas neuronal morphology was unaffected (FIG. 8). We stimulated the neurons by somatic current injection at 50 Hz, which is the mean frequency of regularly spiking pyramidal neurons in the rat hippocampus 19. Delivery of a 20-ms light pulse resulted in suppression of an individual spike in ZipACR-expressing neurons (FIG. 5B, top trace), whereas in GtACR2-expressing cells up to seven successive spikes were suppressed (FIG. 5B, middle trace). These properties make ZipACR a likely first choice for many optogenetic applications, especially in high-frequency neurons.

The sequences encoding the 7TM domains were well expressed in HEK293 cells. These constructs generated photocurrents, and functioned as light-gated anion-conducting channels.

As shown in Table 1, using standard solutions (126 mM KCl in the pipette and 150 mM NaCl in the bath, pH 7.4), GcACR had an action spectra maximum at 486 nm; C1ACR1 had an action spectra maximum at 526 nm; C1ACR2 had an action spectra maximum at 465 nm; R1ACR1 had an action spectra maximum at 519 nm, R1ACR2 had an action spectra maximum at 513 nm, R2ACR1 had an action spectra maximum at 513 nm, and R2ACR2 had an action spectra maximum at 486 nm.

TABLE 1
GenBank accession numbers, organism sources, transcript names, action
spectra maxima and protein name abbreviations of ACR homologs tested
in this study. Three previously known ACRs are also included.

| | | | | Spectral | Protein |
|---|---|---|---|---|---|
| | | | | max. | name |
| # | Accession | Organism | Transcript name | (nm) | abbreviation |
| 1 | KX879674 | Geminigera cryophila | CAMNT | 495 | GcACR_457 |
| | | (CCMP2564) | 0021181457* | | |
| 2 | KX879675 | | CAMNT | 485 | GcACR_145 |
| | | | 0021207145* | | |
| 3 | KX879676 | | CAMNT | 515 | GcACR_439 |
| | | | 0021218439* | | |
| 4 | KX879677 | Geminigera sp. | CAMNT | 475 | G1ACR_203 |
| | | (Caron Lab Isolate) | 0013945203* | | |
| 5 | KX879678 | | CAMNT | N/A | G1ACR_243 |
| | | | 0013979243* | | |
| 6 | KP171708 | Guillardia theta | known previously ⁶ | 515 | GtACR1 |
| 7 | KP171709 | (CCMP2712) | known previously ⁶ | 470 | GtACR2 |
| 8 | KF992074 | Proteomonas sulcata | known previously ⁷ | 520 | PsuACR1 |
| 9 | KX879679 | (CCMP704) | CAMNT | 515 | ZipACR/Psu |
| | | | 0026648973* | | ACR_973 |
| 10 | KX879680 | | CAMNT | 525 | PsuACR_433 |
| | | | 0026650433* | | |
| | KX879681 | Proteomonas sulcata | | | |
| | | (CCMP704) | | | |
| 12 | KX879682 | Rhodomonas lens | CAMNT | 445 | RlACR_477 |
| | | (RHODO) | 0019228477* | | |
| 13 | KX879683 | Rhodomonas salina | CAMNT | 515 | RsACR_995 |
| | | (CCMP1319) | 0012794995* | | |
| 14 | KX879684 | Rhodomonas sp. | CAMNT | 515 | R1ACR_367 |
| | | (CCMP768) | 0042060367* | | |
| 15 | KX879685 | | CAMNT | 520 | R1ACR_877 |
| | | | 0042061877* | | |
| 16 | KX879686 | | CAMNT | 515 | R1ACR_447 |
| | | | 0042066447* | | |
| 17 | KX879687 | | CAMNT | 515 | R1ACR_741 |
| | | | 0049477741* | | |
| 18 | KX879688 | | CAMNT | 520 | R1ACR_799 |
| | | | 0049533799* | | |
| | KX879689 | Rhodomonas sp. | | | |
| | | (CCAC1630) | | | |
| | KX879690 | Rhodomonas sp. | | | |
| | | (CCAC1630) | | | |
| 21 | KX879691 | Chroomonas sp. | CAMNT | 535 | C1ACR_887 |
| | | (CCMP2293) | 0022112887* | | |
| 22 | KX879692 | | CAMNT | 440 | C1ACR_023 |
| | | | 0022176023* | | |
| | | Rhodomonas sp. | IAYV-2004853 | 513 | R2ACR1 |
| | | Rhodomonas sp. | IAYV-2007142 | 486 | R2ACR2 |

*Transcripts from the MMETS project.

TABLE 2
Organism sources and transcript names of
other ACR homologs which may be used.

| Organism | Transcript name |
|---|---|
| Geminigera cryophila CMP2564 | CAMNT_0021207145* |
| Chroomonas sp. CCMP2293 | CAMNT_0022112887* |
| Chroomonas sp. CCMP2293 | CAMNT_0022176023* |
| Rhodomonas sp. CCMP768 | CAMNT_0049533799* |
| Rhodomonas sp. CCMP768 | CAMNT_0042060367* |
| Rhodomonas sp. | IAYV-2004853# |
| Rhodomonas sp. | IAYV-2007142# |
| Rhodomonas sp. CCAC1630) | IAYV-2004853# |
| Rhodomonas sp. CCAC1630 | IAYV-2007142# |
| Proteomonas sulcata CCMP704 | IRZA-2061003# |
| Rhodomonas sp. CCAC1630 | # |
| Rhodomonas sp. CCAC1630 | # |
| Rhodomonas sp. CCAC1630 | # |
| Geminigera cryophila CCMP2564 | CAMNT 0021184197* |
| Geminigera cryophila CCMP2564 | CAMNT 0021188201* |
| Hanusia phi CCMP325 | CAMNT 0009606213* |
| Hemiselmis andersenii CCMP1180 | CAMNT 0009587359* |
| Hemiselmis rufescens PCC563 | CAMNT 0014430495* |
| Proteomonas sulcata(CCMP704 | CAMNT 0026606353* |
| Rhodomonas abbreviate | CAMNT 0023412687* |
| Rhodomonas lens RHODO | CAMNT 0019250497* |
| Rhodomonas salina CCMP1319 | CAMNT 0012765665* |
| Rhodomonas sp. CCMP768 | CAMNT 0049478653* |
| Rhodomonas sp. CCMP768 | CAMNT 0049496367* |
| Rhodomonas sp. CCMP768 | CAMNT 0049531591* |
| Rhodomonas sp. CCMP768 | CAMNT 0049513053* |
| Rhodomonas sp. CCAC1630 | IAYV-2051041# |
| non described CCMP2293 | CAMNT 0022335561* |

Asterisks indicate that the sequence was derived from the MMETS project,
#symbol, from the 1KP project.

TABLE 3
The mean and sem values of the peak current, inactivation (measured as the difference between the
peak value and the value at the end of the 1-s light pulse divided by the peak value, percent) and
decay rate (measured as the reciprocal of the time of the 50% decrease in the current amplitude
after the light off) for ACR homologs tested in this study. For each protein 3-7 cells were measured.
The data for previously characterized GtACR1, GtACR2 and PsuACR1 are also included.

| | Protein | Mean peak | Sem peak | Mean | Sem | Mean decay | Sem decay |
|---|---|---|---|---|---|---|---|
| | name | current | current | inactivation | inactivation | rate | rate |
| # | abbreviation | (nA) | (nA) | (%) | (%) | (ms⁻¹) | (ms⁻¹) |
| 1 | GcACR_457 | 4.8 | 0.4 | 41.7 | 1.3 | 0.0498 | 0.0031 |
| 2 | GcACR_145 | 1.2 | 0.3 | 82.9 | 0.8 | 0.0401 | 0.0016 |
| 3 | GcACR_439 | 1.2 | 0.5 | 31.7 | 3.8 | 0.0298 | 0.0078 |
| 4 | G1ACR_203 | 0.8 | 0.4 | 76.1 | 3.0 | 0.0188 | 0.0029 |
| 5 | G1ACR_243 | no photocurrents | | | | | |
| 6 | GtACR1 | 7.5 | 0.9 | 12.0 | 1.2 | 0.0094 | 0.0014 |
| 7 | GtACR2 | 7.4 | 1.0 | 20.7 | 1.6 | 0.0146 | 0.0017 |
| 8 | PsuACR1 | 0.7 | 0.2 | 43.8 | 1.6 | 0.0485 | 0.0022 |
| 9 | PsuACR_973 | 10.6 | 1.3 | 17.0 | 2.3 | 0.3067 | 0.0591 |
| 10 | PsuACR_433 | 1.2 | 0.1 | 79.5 | 2.2 | 0.0387 | 0.0022 |
| 12 | RlACR_477 | 5.0 | 0.5 | 27.7 | 1.7 | 0.0363 | 0.0042 |
| 13 | RsACR_995 | 3.9 | 1.1 | 32.2 | 3.9 | 0.0740 | 0.0134 |
| 14 | R1ACR_367 | 6.0 | 0.4 | 74.0 | 0.5 | 0.0074 | 0.002 |
| 15 | R1ACR_877 | 1.0 | 0.1 | 12.5 | 1.1 | 0.0087 | 0.0003 |
| 16 | R1ACR_447 | 2.3 | 0.5 | 34.1 | 7.2 | 0.0078 | 0.0033 |
| 17 | R1ACR_741 | 8.8 | 0.9 | 73.6 | 1.3 | 0.0064 | 0.0004 |
| 18 | R1ACR_799 | 0.6 | 0.6 | 73.8 | 5.0 | 0.0037 | 0.002 |
| 21 | C1ACR_887 | 0.05 | 0.04 | 27.5 | 9.3 | 0.125 | 0.02 |
| 22 | C1ACR_023 | 0.6 | 0.5 | 51.5 | 8.9 | 0.029 | 0.0062 |
| 5a | GtACR1_A61E | 0.06 | 0.02 | 24.3 | 10.3 | 0.0073 | 0.0019 |
| 5b | GtACR1_A75E | 0.11 | 0.05 | 11.6 | 1.1 | 0.0030 | 0.0022 |
| 5c | GtACR1_N79E | 6.02 | 4.26 | 12.5 | 0.7 | 0.0093 | 0.0028 |

TABLE 4
The mean and sem values of the peak current, inactivation (measured as the difference between the
peak value and the value at the end of the 1-s light pulse divided by the peak value, percent) and
decay rate (measured as the reciprocal of the time of the 50% decrease in the current amplitude
after the light off) for ACR homologs tested in this study. For each protein 3-7 cells were measured.
The data for previously characterized GtACR1, GtACR2 and PsuACR1 are also included.

| | Protein | Mean peak | Sem peak | Mean | Sem | Mean decay | Sem decay |
|---|---|---|---|---|---|---|---|
| | name | current | current | inactivation | inactivation | rate | rate |
| # | abbreviation | (nA) | (nA) | (%) | (%) | (ms⁻¹) | (ms⁻¹) |
| 1 | GtACR1 | 7.5 | 0.9 | 12.0 | 1.2 | 0.0094 | 0.0014 |
| 2 | GtACR2 | 7.4 | 1.0 | 20.7 | 1.6 | 0.0146 | 0.0017 |
| 3 | PsuACR1 | 0.7 | 0.2 | 43.8 | 1.6 | 0.0485 | 0.0022 |
| 4 | PsuACR_003 | no photocurrents | | | | | |
| 5 | R2ACR_853 | 0.2 | 0.1 | 83.6 | 1.4 | 0.0049 | 0.0018 |
| 6 | R2ACR_142 | 15.0 | 1.6 | 43.5 | 2.7 | 0.0041 | 0.0008 |

TABLE 5
Protein name abbreviations, GenBank accession numbers, source
organisms, transcript names and maxima of photocurrents action
spectra of additional ACR homologs tested in this study.

| | | | | | |
|---|---|---|---|---|---|
| | Protein | | | | Spectral |
| | name | | | | max. |
| # | abbreviation | Accession | Organism | Transcript name | (nm) |

| | | | | |
|---|---|---|---|---|
| 1 | GcACR_197 | Geminigera cryophila | CAMNT 0021184197* | N/A |
| 2 | GcACR_201 | (CCMP2564) | CAMNT 0021188201* | N/A |
| 3 | HpACR_213 | Hanusia phi (CCMP325) | CAMNT 0009606213* | N/A |
| 4 | HaACR_359 | Hemiselmis andersenii | CAMNT 0009587359* | N/A |
| | | (CCMP1180) | | |
| 5 | HrACR_495 | Hemiselmis rufescens | CAMNT 0014430495* | N/A |
| | | (PCC563) | | |
| 6 | PsuACR_353 | Proteomonas sulcata | CAMNT 0026606353* | 520 |
| | | (CCMP704) | | |
| 7 | RaACR_687 | Rhodomonas abbreviata | CAMNT 0023412687* | 515 |
| 8 | RlACR_497 | Rhodomonas lens | CAMNT 0019250497* | 520 |
| | | (RHODO) | | |
| 9 | RsACR_665 | Rhodomonas salina | CAMNT 0012765665* | 520 |
| | | (CCMP1319) | | |
| 10 | R1ACR_653 | Rhodomonas sp. | CAMNT 0049478653* | 470 |
| 11 | R1ACR_6367 | (CCMP768) | CAMNT 0049496367* | 480 |
| 12 | R1ACR_591 | | CAMNT 0049531591* | 520 |
| 13 | R1ACR_053 | | CAMNT 0049513053* | 520 |
| 14 | R2ACR_041 | Rhodomonas sp. | IAYV-2051041^{#} | N/A |
| | | (CCAC1630) | | |
| 15 | C1ACR_561 | non described | CAMNT 0022335561* | N/A |
| | | (CCMP2293) | | |

*Transcripts from the MMETS project.
^{#}Transcripts from the 1KP project.

As natural anion channels, ACRs provide hyperpolarizing and/or depolorizing optogenetic tools optimized by evolution for extremely high light-sensitivity, absolute anion selectivity, and rapid kinetics.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
A. Berndt, S. Y. Lee, C. Ramakrishnan, K. Deisseroth, Science 344, 420 (2014).
A. S. Chuong et al., Nat. Neurosci. 17, 1123 (2014).
B. A. Curtis et al., Nature 492, 59 (2012).
B. Y. Chow et al., Nature 463, 98 (2010).
B. Y. Chow, E. S. Boyden, Sci. Transl. Med. 5, 177ps5 (2013).
D. Gradmann, A. Berndt, F. Schneider, P. Hegemann, Biophys. J. 101, 1057 (2011).
Daniel et al. ATGme: Open-source web application for rare codon identification and custom DNA sequence optimization. BMC Bioinformatics, 16:303, 2015.
E. G. Govorunova, 0. A. Sineshchekov, H. Li, R. Janz, J. L. Spudich, J. Biol. Chem. 288, 29911 (2013).
F. Zhang et al., Cell 147, 1446 (2011).
F. Zhang et al., Nature 446, 633 (2007).
G. Nagel et al., Proc. Natl. Acad. Sci. USA 100, 13940 (2003).
G. Nagel et al., Science 296, 2395 (2002).
Govorunova E G et al. Photochem Photobiol. 2015.
Govorunova E G et al. Science. 349(6248): 647-50, 2015.
Grote et al. JCat: a novel tool to adapt codon usage of a target gene to its potential expression host. Nucleic Acids Research, 33: W526-W531, 2005.
J. L. Spudich, O. A. Sineshchekov, E. G. Govorunova, Biochem. Biophys. Acta 1837, 546 (2014).
J. Wietek et al., Science 344, 409 (2014).
J. Y. Lin, P. M. Knutsen, A. Muller, D. Kleinfeld, R. Y. Tsien, Nat. Neurosci. 16, 1499 (2013).
K. Deisseroth, Nat. Methods 8, 26 (2011).
K. Eisenhauer et al., J. Biol. Chem. 287, 6904 (2012).
K. Ruffert et al., Biochem. Biophys. Res. Commun. 410, 737 (2011).
M. Kolbe, H. Besir, L. O. Essen, D. Oesterhelt, Science 288, 1390 (2000).
N. C. Klapoetke et al., Nat. Methods 11, 338 (2014).
O. A. Sineshchekov et al., Biophys. J. 89, 4310 (2005).
O. A. Sineshchekov, K.-H. Jung, J. L. Spudich, Proc. Natl. Acad. Sci. USA 99, 8689 (2002).
O. P. Ernst et al., Chem. Rev. 114, 126 (2014).
P. Bregestovski, T. Waseem, M. Mukhtarov, Front. Mol. Neurosci. 2, 15 (2009).
S. J. Husson et al., PLoS One 7, e40937 (2012).
Sineshchekov O A et al. Proc Natl Acad Sci USA. 112 (46):14236-41, 2015.
T. J. Jentsch, V. Stein, F. Weinreich, A. A. Zdebik, Physiol Rev. 0.82, 503 (2002).
U.S. Patent Application No. 62/261,821.
V. Gradinaru et al., Cell 141, 154 (2010).
V. Gradinaru, K. R. Thompson, K. Deisseroth, Brain Cell Biol. 36, 129 (2008).
X. Han, E. S. Boyden, PLoS One 2, e299 (2007).
1 Deisseroth, K. Optogenetics: 10 years of microbial opsins in neuroscience. Nat. Neurosci. 18, 1213-1225 (2015).
2 Zhang, F. et al. Multimodal fast optical interrogation of neural circuitry. Nature 446, 633-639 (2007).
3 Chow, B. Y. et al. High-performance genetically targetable optical neural silencing by light-driven proton pumps. Nature 463, 98-102 (2010).
4 Wietek, J. et al. An improved chloride-conducting channelrhodopsin for light-induced inhibition of neuronal activity in vivo. Sci. Rep. 5, 14807 (2015).
5 Berndt, A. et al. Structural foundations of optogenetics: Determinants of channelrhodopsin ion selectivity. Proc. Natl. Acad. Sci. USA 113, 822-829 (2015).
6 Govorunova, E. G., Sineshchekov, O. A., Liu, X., Janz, R. & Spudich, J. L. Natural light-gated anion channels: A family of microbial rhodopsins for advanced optogenetics. Science 349, 647-650 (2015).
7 Govorunova, E. G., Sineshchekov, O. A. & Spudich, J. L. Proteomonas sulcata ACR1: A fast anion channelrhodopsin. Photochem. Photobiol. 92, 257-263 (2016).
8 Wietek, J., Broser, M., Krause, B. S. & Hegemann, P. Identification of a natural green light absorbing chloride conducting channelrhodopsin from Proteomonas sulcata. J. Biol. Chem. 291, 4121-4127 (2016).
9 Keeling, P. J. et al. The Marine Microbial Eukaryote Transcriptome Sequencing Project (MMETSP): illuminating the functional diversity of eukaryotic life in the oceans through transcriptome sequencing. PLoS Biol. 12, e1001889 (2014).
10 Matasci, N. et al. Data access for the 1,000 Plants (1KP) project. Gigascience 3, 17 (2014).
11 Govorunova, E. G., Spudich, E. N., Lane, C. E., Sineshchekov, O. A. & Spudich, J. L. New channelrhodopsin with a red-shifted spectrum and rapid kinetics from Mesostigma viride. mBio 2, e00115-00111 (2011).
12 Sineshchekov, O. A., Jung, K.-H. & Spudich, J. L. Two rhodopsins mediate phototaxis to low- and high-intensity light in Chlamydomonas reinhardtii. Proc. Natl. Acad. Sci. USA 99, 8689-8694 (2002).
13 Nagel, G. et al. Channelrhodopsin-1: a light-gated proton channel in green algae. Science 296, 2395-2398 (2002).
14 Nagel, G. et al. Channelrhodopsin-2, a directly light-gated cation-selective membrane channel. Proc. Natl. Acad. Sci. USA 100, 13940-13945 (2003).
15 Sineshchekov, O. A., Govorunova, E. G., Li, H. & Spudich, J. L. Gating mechanisms of a natural anion channelrhodopsin. Proc. Natl. Acad. Sci. USA 112, 14236-14241 (2015).
16 Wietek, J. et al. Conversion of channelrhodopsin into a light-gated chloride channel. Science 344, 409-412 (2014).
17 Kato, H. E. et al. Atomistic design of microbial opsin-based blue-shifted optogenetics tools. Nat. Commun. 6, 7177 (2015).
18 Berndt, A. & Deisseroth, K. Expanding the optogenetics toolkit. Science 349, 590-591 (2015).
19 Graves, A. R. et al. Hippocampal pyramidal neurons comprise two distinct cell types that are counter modulated by metabotropic receptors. Neuron 76, 776-789 (2012).
20 Hou, S. Y. et al. Diversity of Chlamydomonas channelrhodopsins. Photochem. Photobiol. 88, 119-128 (2012).
21 Klapoetke, N. C. et al. Independent optical excitation of distinct neural populations. Nat. Methods 11, 338-346 (2014).
22 Berndt, A., Lee, S. Y., Ramakrishnan, C. & Deisseroth, K. Structure-guided transformation of channelrhodopsin into a light-activated chloride channel. Science 344, 420-424 (2014).
23 Sineshchekov, 0. A., Li, H., Govorunova, E. G. & Spudich, J. L. Photochemical reaction cycle transitions during anion channelrhodopsin gating. Proc. Natl. Acad. Sci. USA 113, E1993-2000 (2016).
24 Yi, A., Mamaeva, N. V., Li, H., Spudich, J. L. & Rothschild, K. J. Resonance Raman study of an anion channelrhodopsin: Effects of mutations near the retinylidene Schiff base. Biochemistry 55, 2371-2380 (2016).
25 Ogren, J. I. et al. Proton transfers in a Channelrhodopsin-1 studied by FTIR-difference spectroscopy and site-directed mutagenesis. J. Biol. Chem. 290, 12719-12730 (2015).
26 Verhoefen, M. K. et al. The photocycle of channelrhodopsin-2: Ultrafast reaction dynamics and subsequent reaction steps. Chemphyschem 11, 3113-3122 (2010).
27 Gunaydin, L. A. et al. Ultrafast optogenetic control. Nat. Neurosci. 13, 387-392 (2010).
28 Gradinaru, V., Thompson, K. R. & Deisseroth, K. eNpHR: a Natronomonas halorhodopsin enhanced for optogenetic applications. Brain Cell Biol. 36, 129-139 (2008).
29 Mahn, M., Prigge, M., Ron, S., Levy, R. & Yizhar, O. Biophysical constraints of optogenetic inhibition at presynaptic terminals. Nat. Neurosci. 19, 554-556 (2016).
30 Govorunova, E. G., Cunha, C. R., Sineshchekov, O. A. & Spudich, J. L. Anion channelrhodopsins for inhibitory cardiac optogenetics. Sci. Rep. 6, 33530 (2016).
31 Eddy, S. R. Accelerated profile HMM searches. PLoS Comput. Biol. 7, e1002195 (2011).
32 Larsson, A. AliView: a fast and lightweight alignment viewer and editor for large datasets. Bioinformatics 30, 3276-3278 (2014).
33 Gouy, M., Guindon, S. & Gascuel, O. SeaView version 4: A multiplatform graphical user interface for sequence alignment and phylogenetic tree building. Mol. Biol. Evol. 27, 221-224 (2010).
34 Katoh, K. & Standley, D. M. MAFFT multiple sequence alignment software version 7: improvements in performance and usability. Mol. Biol. Evol. 30, 772-780 (2013).
35 Lois, C., Hong, E. J., Pease, S., Brown, E. J. & Baltimore, D. Germline transmission and tissue-specific expression of transgenes delivered by lentiviral vectors. Science 295, 868-872 (2002).
36 Hosaka, M., Hammer, R. E. & Südhof, T. C. A phospho-switch controls the dynamic association of synapsins with synaptic vesicles. Neuron 24, 377-387 (1999).
37 Ippolito, D. M. & Eroglu, C. Quantifying synapses: an immunocytochemistry-based assay to quantify synapse number. J. Vis. Exp. (2010).

What is claimed is:

1. A recombinant nucleic acid operatively linked to a heterologous promoter sequence, said recombinant nucleic acid comprising:
(i) a sequence encoding an anion-conducting channelrhodopsin that is at least about 90% identical to the polypeptide sequence encoded by SEQ ID NO: 31, SEQ ID NO: 2, SEQ ID NO: 4, SEQ ID NO: 6, SEQ ID NO: 8, SEQ ID NO: 10-30, SEQ ID NO: 32-45 or SEQ ID NO: 70-95
(ii) a sequence encoding an anion-conducting channelrhodopsin at least about 90% identical to a sequence selected from SEQ ID NO: 1, SEQ ID NO: 3, SEQ ID NO: 5, SEQ ID NO: 7, SEQ ID NO: 9, SEQ ID NO: 56-68 or SEQ ID NO: 105-118; or
(iii) a sequence that is at least about 90% identical to a sequence selected from the group consisting of SEQ ID NO: 2, SEQ ID NO: 4, SEQ ID NO: 6, SEQ ID NO: 8, SEQ ID NO: 10-45 or SEQ ID NO: 70-95 and encodes an anion-conducting channelrhodopsin.

2. The recombinant nucleic acid of claim 1, wherein it comprises an expression vector.

3. A recombinant host cell comprising a recombinant nucleic acid of claim 1.

4. The recombinant host cell of claim 3, wherein said host cell is an isolated human cell.

5. The recombinant host cell of claim 3, wherein said host cell is a non-human mammalian cell.

6. The recombinant host cell of claim 3, wherein said host cell is a bacterial cell.

7. The recombinant host cell of claim 3, wherein said host cell is a yeast cell.

8. The recombinant host cell of claim 3, wherein said host cell is an insect cell.

9. The recombinant host cell of claim 3, wherein said host cell is a plant cell.

10. The recombinant host cell of claim 3, wherein said host cell is an isolated neuronal cell.

11. The recombinant host cell of claim 3, wherein said host cell is an isolated electrically active cell.

12. A method of membrane potential photocontrol, including hyperpolarization of a cell in a subject suffering from a neuron mediated disorder, said method comprising:
(a) delivering to the cell of said subject an expression vector encoding:
(i) a sequence encoding an anion-conducting channelrhodopsin that is at least about 90% identical to the polypeptide sequence encoded by SEQ ID NO: 31, SEQ ID NO: 2, SEQ ID NO: 4, SEQ ID NO: 6, SEQ ID NO: 8, SEQ ID NO: 10-30, SEQ ID NO: 32-45 or SEQ ID NO: 70-95
(ii) a sequence encoding an anion-conducting channelrhodopsin at least about 90% identical to a sequence selected from SEQ ID NO: 1, SEQ ID NO: 3, SEQ ID NO: 5, SEQ ID NO: 7, SEQ ID NO: 9, SEQ ID NO: 56-68 or SEQ ID NO: 105-118; or
(iii) a sequence that is at least about 90% identical to a sequence selected from the group consisting of SEQ ID NO: 2, SEQ ID NO: 4, SEQ ID NO: 6, SEQ ID NO: 8, SEQ ID NO: 10-45 or SEQ ID NO: 70-95 and encodes an anion-conducting channelrhodopsin; and
(b) expressing said vector in said cell, wherein the expressed rhodopsin silences the signal from said neuron or in the case of neurons with high cytoplasmic chloride concentration, wherein the expressed rhodopsin depolarizes the plasma membrane.

13. The method of claim 12, said method comprising:
(a) delivering to the cell of said subject an expression vector comprising a polynucleotide sequence at least about 90% identical to a sequence selected from the group consisting of SEQ ID NO: 2, SEQ ID NO: 4, SEQ ID NO: 6, SEQ ID NO: 8, SEQ ID NO: 10-45 or SEQ ID NO: 70-95 which encodes an anion-conducting rhodopsin domain expressible in said cell; and
(b) expressing said vector in said cell, wherein the expressed rhodopsin silences the signal from said neuron, or in the case of neurons with high cytoplasmic chloride concentration, wherein the expressed rhodopsin depolarizes the plasma membrane.

14. A method of neuronal silencing in a subject suffering from a neuron mediated disorder, said method comprising:
(a) delivering to a target neuron of said subject an expression vector encoding:
(i) a sequence encoding an anion-conducting channelrhodopsin that is at least about 90% identical to the polypeptide sequence encoded by SEQ ID NO: 31, SEQ ID NO: 2, SEQ ID NO: 4, SEQ ID NO: 6, SEQ ID NO: 8, SEQ ID NO: 10-30, SEQ ID NO: 32-45 or SEQ ID NO: 70-95
(ii) a sequence encoding an anion-conducting channelrhodopsin at least about 90% identical to a sequence selected from SEQ ID NO: 1, SEQ ID NO: 3, SEQ ID NO: 5, SEQ ID NO: 7, SEQ ID NO: 9, SEQ ID NO: 56-68 or SEQ ID NO: 105-118; or
(iii) a sequence that is at least about 90% identical to a sequence selected from the group consisting of SEQ ID NO: 2, SEQ ID NO: 4, SEQ ID NO: 6, SEQ ID NO: 8, SEQ ID NO: 10-45 or SEQ ID NO: 70-95 and encodes an anion-conducting channelrhodopsin; and
(b) expressing said vector in said target neuron, wherein the expressed rhodopsin results in silencing of the signal from the target neuron or in the case of neurons with high cytoplasmic chloride concentration, wherein the expressed rhodopsin depolarizes the plasma membrane.

15. The method of claim 14 comprising:
(a) delivering to a target neuron of said subject an expression vector comprising a polynucleotide that encodes a nucleic acid sequence at least about 90% identical to a sequence selected from the group consisting of SEQ ID NO: 2, SEQ ID NO: 4, SEQ ID NO: 6, SEQ ID NO: 8, SEQ ID NO: 10-45 or SEQ ID NO: 70-95, which encodes a rhodopsin domain of an anion-conducting channelrhodopsin expressible in said target neuron;
(b) expressing said vector in said target neuron, wherein the expressed rhodopsin results in silencing of the signal from the target neuron, or in the case of neurons with high cytoplasmic chloride concentration, wherein the expressed rhodopsin depolarizes the plasma membrane.

16. A method of restoring photosensitivity to a retina of a subject suffering from vision loss or blindness, said method comprising:
(a) delivering to the retina of said subject an expression vector vector encoding:
(i) a sequence encoding an anion-conducting channelrhodopsin that is at least about 90% identical to the polypeptide sequence encoded by SEQ ID NO: 31, SEQ ID NO: 2, SEQ ID NO: 4, SEQ ID NO: 6, SEQ ID NO: 8, SEQ ID NO: 10-30, SEQ ID NO: 32-45 or SEQ ID NO: 70-95
(ii) a sequence encoding an anion-conducting channelrhodopsin at least about 90% identical to a sequence selected from SEQ ID NO: 1, SEQ ID NO: 3, SEQ ID NO: 5, SEQ ID NO: 7, SEQ ID NO: 9, SEQ ID NO: 56-68 or SEQ ID NO: 105-118; or
(iii) a sequence that is at least about 90% identical to a sequence selected from the group consisting of SEQ ID NO: 2, SEQ ID NO: 4, SEQ ID NO: 6, SEQ ID NO: 8, SEQ ID NO: 10-45 or SEQ ID NO: 70-95 and encodes an anion-conducting channelrhodopsin; and
(b) expressing said vector in said retinal neuron, wherein the expressed rhodopsin renders a high level of membrane potential in said retinal neuron, or in the case of neurons with high cytoplasmic chloride concentration, wherein the expressed rhodopsin depolarizes the plasma membrane.

17. The method of claim 16, comprising:
(a) delivering to the retina of said subject an expression vector comprising a polynucleotide that encodes a nucleic acid sequence at least about 90% identical to a sequence selected from the group consisting of SEQ ID NO: 2, SEQ ID NO: 4, SEQ ID NO: 6, SEQ ID NO: 8, SEQ ID NO: 10-45 or SEQ ID NO: 70-95, which encodes a rhodopsin domain of an anion-conducting channelrhodopsin expressible in a retinal neuron; and
(b) expressing said vector in said retinal neuron, wherein the expressed rhodopsin renders a high level of membrane potential in said retinal neuron, or in the case of neurons with high cytoplasmic chloride concentration, wherein the expressed rhodopsin depolarizes the plasma membrane.

18. The method of claim 12 wherein, said method comprises:
(a) delivering to the cell of said subject an expression vector that encodes a rhodopsin domain; said vector comprising an open reading frame encoding a nucleic acid sequence at least about 90% identical to a sequence selected from the group consisting of SEQ ID NO: 2, SEQ ID NO: 4, SEQ ID NO: 6, SEQ ID NO: 8, SEQ ID NO: 10-45 or SEQ ID NO: 70-95, operatively linked to a promoter sequence; and
(b) expressing said vector in said cell, wherein the expressed rhodopsin renders a high level of membrane potential in said cell.

19. The method of claim 12 wherein, said method comprises:
(a) delivering to the cell of said subject an expression vector that encodes a rhodopsin domain; said vector comprising an open reading frame encoding the rhodopsin domain of an anion-conducting channelrhodopsin selected from SEQ ID NO: 1, SEQ ID NO: 3, SEQ ID NO: 5, SEQ ID NO: 7, SEQ ID NO: 9, SEQ ID NO: 56-68 or SEQ ID NO: 105-118, operatively linked to a promoter sequence; and
(b) expressing said vector in said cell, wherein the expressed rhodopsin renders a high level of membrane potential in said cell.

20. The method of claim 14, wherein the nucleic acid sequence at least about 90% identical to a sequence selected from the group consisting of SEQ ID NO: 2, SEQ ID NO: 4, SEQ ID NO: 6, SEQ ID NO: 8, SEQ ID NO: 10-45 or SEQ ID NO: 70-95, or the complement thereof, further comprises hybridization to filter-bound DNA in 0.5 M NaHPO₄, 7% sodium dodecyl sulfate, 1 mM EDTA at 65° C., and washing in 0.2×SSC/0.1% SDS at 42° C.

21. The method of claim 16, wherein said subject is mammalian.

22. The method of claim 16, wherein said subject is human.

23. The method of claim 16, wherein said delivering comprises a pharmaceutically acceptable carrying agent.

24. An isolated nucleic acid comprising a nucleic acid sequence that encodes a nucleic acid sequence at least about 90% identical to a sequence selected from the group consisting of SEQ ID NO: 2, SEQ ID NO: 4, SEQ ID NO: 6, SEQ ID NO: 8, SEQ ID NO: 10-45 or SEQ ID NO: 70-95.

25. A method of treating a disorder in an electrically active cell in a subject suffering from a disorder that involves electrically active cells, said method comprising:
(a) delivering to the cell of said subject an expression vector comprising a polynucleotide having a nucleic acid sequence at least about 90% identical to a sequence selected from the group consisting of SEQ ID NO: 2, SEQ ID NO: 4, SEQ ID NO: 6, SEQ ID NO: 8, SEQ ID NO: 10-45 or SEQ ID NO: 70-95, expressible in said cell; and
(b) expressing said vector in said electrically active cell, wherein the expressed rhodopsin silences the signal from said electrically active cell.

26. A method of treating a disorder in an electrically active cell in a subject suffering from a disorder that involves electrically active cells, said method comprising:
(a) delivering to said subject a transgenic cell comprising an expression vector comprising a polynucleotide that encodes a nucleic acid sequence at least about 90% identical to a sequence selected from the group consisting of SEQ ID NO: 2, SEQ ID NO: 4, SEQ ID NO: 6, SEQ ID NO: 8, SEQ ID NO: 10-45 or SEQ ID NO: 70-95, expressible in said transgenic cell; and
(b) expressing said vector in said transgenic cell, wherein the expression silences the signal from a electrically active cell.

27. A method of silencing an electrically active cell in a subject suffering from an electrically active cell mediated disorder, said method comprising:
(a) delivering to a target neuron of said subject an expression vector comprising a polynucleotide that encodes a nucleic acid sequence at least about 90% identical to a sequence selected from the group consisting of SEQ ID NO: 2, SEQ ID NO: 4, SEQ ID NO: 6, SEQ ID NO: 8, SEQ ID NO: 10-45 or SEQ ID NO: 70-95, which encodes a rhodopsin domain of an anion-conducting channelrhodopsin expressible in said target neuron; and
(b) expressing said vector in said target electrically active cell, wherein the expressed rhodopsin results in silencing of the signal from the electrically active cell.

28. An isolated nucleic acid molecule comprising a sequence encoding an anion-conducting channelrhodopsin having a sequence at least about 90% identical to a sequence according to SEQ ID NO: 2, SEQ ID NO: 4, SEQ ID NO: 6, SEQ ID NO: 8, SEQ ID NO: 10-45 or SEQ ID NO: 70-95.

29. The isolated nucleic acid molecule of claim 28, comprising a sequence having a sequence at least about 95% identical to a sequence according to SEQ ID NO: 2, SEQ ID NO: 4, SEQ ID NO: 6, SEQ ID NO: 8, SEQ ID NO: 10-45 or SEQ ID NO: 70-95.

30. The isolated nucleic acid molecule of claim 28, where the nucleic acid is a DNA.

31. The isolated nucleic acid molecule of claim 28, where the nucleic acid is a RNA.

32. The isolated nucleic acid molecule of claim 31, where the nucleic acid is a mRNA.

33. An expression vector comprising a nucleic acid molecule according to claim 28.

34. A recombinant host cell comprising a nucleic acid of claim 28.

35. The recombinant host cell of claim 34, wherein said host cell is an isolated human cell.

36. The recombinant host cell of claim 34, wherein said host cell is a non-human mammalian cell.

37. The recombinant host cell of claim 34, wherein said host cell is a bacterial cell.

38. The recombinant host cell of claim 34, wherein said host cell is a yeast cell.

39. The recombinant host cell of claim 34, wherein said host cell is an insect cell.

40. The recombinant host cell of claim 34, wherein said host cell is a plant cell.

41. The recombinant host cell of claim 34, wherein said host cell is an isolated neuronal cell.

42. The recombinant host cell of claim 34, wherein said host cell is an isolated electrically active cell.

43. A method of treating a subject suffering from a disorder that involves electrically active cells comprising expressing in the subject an effective amount of an anion-conducting channelrhodopsin at the site of the electrically active cells.

44. The method of claim 43, wherein the subject is suffering from neuropathic pain the method comprising expressing in the subject an effective amount of an anion-conducting channelrhodopsin at the site of the pain.

45. The method of claim 43, wherein said expressing comprises administering an anion-conducting channelrhodopsin to the subject.

46. The method of claim 45, wherein the anion-conducting channelrhodopsin further comprises a cell-penetrating peptide (CPP) sequence or a cellular receptor-binding sequence.

47. The method of claim 43, wherein said expressing comprises administering a vector encoding an anion-conducting channelrhodopsin to the subject.

48. The method of claim 47, wherein the vector is a RNA vector.

49. The method of claim 47, wherein the vector is a DNA vector.

50. The method of claim 49, wherein the sequence encoding the anion-conducting channelrhodopsin is operably linked to a heterologous promoter.

51. The method of claim 50, wherein the promoter is an inducible or a repressible promoter.

52. The method of claim 50, wherein the promoter is a tissue or cell type specific promoter.

53. The method of claim 52, wherein the promoter is neuronal cell specific promoter.

54. The method of claim 49, wherein the vector is a plasmid, a viral vector or an episomal vector.

55. The method of claim 49, wherein the vector further comprises an inducible expression cassette for a suicide gene.

56. The method of claim 43, wherein the subject has an amputated limb, diabetes, multiple sclerosis or has undergone a surgery.

57. The method of claim 43, wherein the anion-conducting rhodopsin domain comprises an nucleic acid sequence at least about 90% identical to a sequence of SEQ ID NO: 2, SEQ ID NO: 4, SEQ ID NO: 6, SEQ ID NO: 8, SEQ ID NO: 10-45 or SEQ ID NO: 70-95.

58. The method of claim 47, wherein the anion-conducting rhodopsin domain is encoded by a sequence at about 90% identical to sequence according to SEQ ID NO: 2, SEQ ID NO: 4, SEQ ID NO: 6, SEQ ID NO: 8, SEQ ID NO: 10-45 or SEQ ID NO: 70-95.


SEQUENCE LISTING (118 sequences)

SEQ ID NO: 1 - protein, 326 aa, Artificial Sequence
  synthetic rhodopsin (7 TM) domain (GcACR_145)
MLDAQDTGSQAVGFQPEERMVVRGKAAYIDGIGPATPYAGMYGEWEIANPDCVNKGLHATSRERTIITLWAVVCLCQALFMMTRAPKVTWEAVYLPLVECILYCCTGAGYGVLRMGDGRMLPLSRMCSWLITCPIMLSQAFGIHDMKLYGIQLRMPICAASLIRTVCGLLASTTTDTKIAKWLFFFLGFSFYTMEITTVWFIFQQGIAKFTPTTDVPENATALRRLYFMRFIFFTSWTAFPVFWVLSSTGLCVISEQTSALGYLCADALCKNLYGVIQTNTVWGLLGGTWTPKKHPNNHDEEAPANSHNHKEEDTFAAPHGRFNSA

SEQ ID NO: 2 - DNA, 978 bp, Artificial Sequence
  cDNA synthetic humanized rhodopsin (7 TM) domain (GcACR_145-1)
atgctcgacgctcaggacaccggcagccaagccgtgggattccagcctgaagagagaatggtggtcaggggaaaggccgcctacatcgatggcatcggccctgccaccccttacgctggcatgtacggcgagtgggagatcgccaaccccgattgcgtgaacaagggcctgcacgccaccagcagggagagaacaatcatcacactctgggccgtggtgtgcctctgtcaggccctgttcatgatgaccagggcccccaaggtgacctgggaagccgtgtacctgcccctggtggagtgcatcctgtactgctgcaccggcgccggatacggagtcctcaggatgggcgacggcagaatgctgcccctgtccagaatgtgtagctggctgatcacctgtcccatcatgctgagccaggctttcggcatccacgacatgaagctctacggcatccagctgaggatgcccatctgcgccgccagcctgatcaggaccgtgtgtggcctgctggccagcaccaccaccgacaccaagatcgccaagtggctgttcttcttcctgggcttcagcttctacaccatggagatcaccacagtgtggttcatcttccagcagggcatcgccaagttcacccccaccaccgacgtccctgaaaacgccacagccctgaggaggctgtacttcatgaggttcatcttcttcacctcctggaccgctttccctgtgttttgggtgctgagctccaccggcctctgtgtgatctccgaacagacatccgccctgggctacctgtgcgccgacgccctgtgcaagaacctgtatggcgtcatccagaccaacaccgtgtggggcctcctgggcggaacatggacccccaagaagcaccccaacaaccacgacgaagaggcccccgccaatagccacaaccacaaggaggaggacaccttcgccgcccctcatggcagattcaacagcgcc

SEQ ID NO: 3 - protein, 329 aa, Artificial Sequence
  synthetic rhodopsin (7 TM) domain (C1ACR_887)
MEHHSYDGAMRSLQAISGSFADIEGHWNGPTSVLDNPAVIDGQLYGAYVFMHPECHQGEYAPIVKIIQLCWAVICMCQVLFMFSRAPRVPWEAMYLPGVECVIYTLAFTGNGYVRMVDGRILTWSRLASWLVCCPVMLGQISAITLIKYQSIPLNPMMIACSIIRTVFGVSAGVVQEEPLKWVFFWLGIFFFAFELLGCYLIFAIAIADFEACKTELANKVIGRIKTMRAIIFICWPAITVTWVLSSAGTCLVDDNYTTIGFVVLDAVCKNMYGVVIWSTTWGLLNGKWDRDYARTFGQEEEQQEEAPDFDAKGGAMPGANDNVEVKMF

SEQ ID NO: 4 - DNA, 987 bp, Artificial Sequence
  cDNA synthetic humanized rhodopsin (7 TM) domain (C1ACR_887-1)
atggagcatcacagctacgatggcgccatgaggagcctgcaggctatcagcggctccttcgccgacatcgagggacactggaacggccccacctccgtgctggataaccccgccgtgatcgacggacagctgtacggagcctatgtcttcatgcaccctgagtgccaccagggcgagtacgcccccattgtgaagatcatccagctgtgctgggccgtgatctgcatgtgccaagtgctgttcatgttcagcagggcccctagggtgccctgggaggccatgtatctgcccggcgtggagtgcgtgatctacaccctggccttcacaggcaacggctacgtgaggatggtggacggcagaatcctgacctggagcagactggccagctggctggtgtgttgccctgtgatgctgggccagatctccgccatcaccctgatcaagtaccagtccatccctctgaaccccatgatgatcgcctgcagcatcattaggaccgtgtttggcgtgagcgctggcgtggtgcaagaggagcccctgaagtgggtgttcttctggctgggcatcttctttttcgccttcgagctgctgggctgctacctgattttcgctatcgccatcgccgacttcgaggcctgcaagaccgaactggccaacaaagtgatcggcagaatcaagaccatgagggccatcatcttcatctgctggcccgccatcaccgtgacatgggtgctgtcctccgccggcacctgtctggtggacgacaactacaccaccatcggcttcgtcgtgctggacgctgtgtgcaagaacatgtacggcgtcgtcatctggagcaccacctggggcctcctgaacggcaagtgggacagagactacgccagaaccttcggccaggaggaagaacagcaggaggaagcccctgactttgacgccaagggaggagccatgcctggcgccaacgacaatgtggaggtgaagatgttc

SEQ ID NO: 5 - protein, 300 aa, Artificial Sequence
  synthetic rhodopsin (7 TM) domain (C1ACR_23)
MTVLEVNEAVIIDGVMYGEYIFSHPQCIPAELVPVVKVFQICWAVVSMCQSAFMMTRMPKVPWEAIYLPMVEAIVYVMAYTGNGYLKMGSGRVLPWARMASWLVTCPVMHGQISGMTLIKYQSIPINPMVIAASIIRVVMGVTATIVVTEDLKWAFFGCGLVCLLFEYASVYTIFAVTIGDFADAKTDLGNQVVARLKSIRSIFFVAWTAFPILWLLSSTGTCVIDENISAVAYLISDAFCKNFNGILLWSTTWGLLNGKWDRDFVRAPPAAESEKDVQDAPRPKSDVEVKMFGKVIGSV

SEQ ID NO: 6 - DNA, 903 bp, Artificial Sequence
  cDNA synthetic humanized rhodopsin (7 TM) domain (C1ACR_23-1)
atgaccgtgctggaagtgaacgaagccgtgatcatcgacggagtgatgtacggcgaatacatcttctcccatccccagtgtatccctgccgagctcgtgcccgtggtgaaggtgtttcagatctgctgggccgtggtgagcatgtgccagtccgccttcatgatgaccagaatgcccaaggtgccctgggaagccatctacctgcccatggtggaggccatcgtctacgtcatggcctacacaggaaacggctacctgaagatgggctccggcagagtgctgccttgggccaggatggccagctggctggtgacctgccccgtgatgcacggacagatctccggcatgaccctgatcaagtaccagagcatccctatcaaccccatggtcatcgccgccagcatcatcagagtggtgatgggcgtgaccgctaccatcgtggtgaccgaagacctcaagtgggccttcttcggctgcggactggtctgcctgctgttcgagtacgccagcgtgtacaccatcttcgccgtgaccattggcgactttgccgacgccaagaccgatctgggcaaccaggtggtggccaggctgaagagcatcaggagcatcttcttcgtcgcctggaccgccttccccattctgtggctcctgagcagcaccggcacatgcgtgatcgacgagaacatcagcgccgtggcctacctgattagcgacgccttctgcaagaacttcaacggaatcctgctgtggtccaccacctggggcctgctgaacggcaagtgggatagggacttcgtgagggcccctcctgctgccgaatccgagaaggacgtgcaggacgcccccagacccaagagcgacgtggaggtgaagatgttcggcaaggtgatcggcagcgtgagg

SEQ ID NO: 7 - protein, 303 aa, Artificial Sequence
  synthetic rhodopsin (7 TM) domain (R1ACR_799)
MAGVQDDWSAVSVPAFGQYGTWEAANPECVEMGLSTSKNTKMIQLLFASICACQVLFMASRAPRVTWEAVYLPLVETILYGMASTGNGSMRLADGRMVPIARFCSWIITCPIMLFQVVGIHEVKVYGISCKNMVMAASLVRTVYGIGASVTIDDKMRWIQYMLSCFTFLFELWCVYQIYNAGLKKFSAVKTPLNNIVHSRIQFLRFLFYLSWSSFPIVWVLSSTGLCLINEDGSVLAYLVADILCKNTYGIINANTLFRVLDGKWTPETLEEAANTYGIINANTLFRVLNGKWEPETEEEAKN

SEQ ID NO: 8 - DNA, 909 bp, Artificial Sequence
  cDNA synthetic humanized rhodopsin (7 TM) domain (R1ACR_799-1)
atggccggcgtgcaggatgactggagcgctgtgagcgtgcctgccttcggccagtatggcacatgggaagctgccaaccctgagtgcgtcgagatgggcctgagcaccagcaagaacaccaagatgatccagctgctgttcgccagcatttgtgcctgtcaggtgctgttcatggcctccagagcccccagagtgacctgggaggccgtgtacctgcccctggtggaaaccatcctgtatggcatggcctccaccggcaacggctccatgaggctggctgacggcagaatggtgcccatcgccaggttctgcagctggatcatcacctgccccatcatgctctttcaggtggtgggcattcacgaggtgaaagtgtacggcatttcctgcaaaaacatggtgatggccgccagcctggtgaggaccgtgtacggcatcggagcctccgtcaccatcgacgacaaaatgaggtggattcaatacatgctgtcctgcttcaccttcctgttcgagctgtggtgcgtgtaccagatctacaacgccggcctgaagaagttcagcgccgtcaagacccccctgaataacatcgtgcactcccgtattcaattcctgaggttcctcttctacctgagctggagcagcttccccatcgtgtgggtgctgagcagcaccggactgtgcctgatcaacgaggacggctccgtgctggcttacctggtcgccgatatcctgtgcaagaacacctacggaatcatcaacgctaataccctgttcagggtgctggatggcaaatggacacccgagaccctggaggaggccgccaacacctacggcattatcaacgccaacaccctgtttagagtgctgaacggaaagtgggagcccgagaccgaggaggaagctaaaaac

SEQ ID NO: 9 - protein, 294 aa, Artificial Sequence
  synthetic rhodopsin (7 TM) domain (R1ACR_367)
MSYVEDPAFGFYGSWEAANQDCIERGLSTTNNTRMIQLLFASICACQVLFMASRAPRVTWEAVYLPVVETVLYGMASTGNGSMRLADGRIVPIARFCSWLITCPIMLFQVVGIHELKWRTISCKNMVMAASLIRTVFGIGASVTITPTMRWVQYFLSLSFFLFELIVVYQIFSAGLKKFGAVKTSLNNIVYSRIMFLRFLFFLSWSSFPIIWLLSSTGFCLVNEDVSVLAYLVADMICKNLYGITNANTLFRVLNGKWTPETEEEAKQPKDNEHAHVGETYFEQMLSGLRGSRH

SEQ ID NO: 10 - DNA, 882 bp, Artificial Sequence
  cDNA synthetic humanized construct R1ACR_367-1
atgagctacgtggaggaccctgccttcggcttctacggctcctgggaggccgccaaccaggactgtattgaaaggggcctcagcaccaccaacaataccaggatgatccagctgctgttcgcctccatctgcgcctgccaggtcctgtttatggccagcagggcccctagggtgacatgggaggccgtgtacctgcccgtggtggagaccgtgctgtacggcatggccagcaccggcaacggcagcatgagactggccgacggcaggatcgtgcctatcgccaggttctgtagctggctgatcacctgccccattatgctgttccaggtcgtgggcatccacgagctgaagtggaggaccatcagctgcaagaacatggtgatggccgccagcctgatcagaaccgtgtttggcatcggcgcctccgtgacaattacccccaccatgagatgggtgcagtacttcctgtccctgtccttcttcctgttcgagctgatcgtggtctaccagatcttcagcgccggcctgaagaaattcggcgccgtgaagacaagcctgaacaacatcgtctacagcaggatcatgttcctgaggttcctgttcttcctgagctggtccagcttccccatcatctggctgctgtccagcaccggcttttgtctggtgaacgaggacgtgagcgtgctggcctacctggtcgctgacatgatctgcaagaacctgtacggcatcaccaatgccaacaccctgttcagggtgctcaacggcaagtggacccctgagacagaggaggaggccaagcagcctaaggacaacgagcacgcccatgtgggcgagacctatttcgagcagatgctgagcggcctgagaggaagcaggcac

SEQ ID NO: 11 - DNA, 978 bp, Artificial Sequence
  cDNA synthetic humanized construct GcACR_145-2
atgctggacgcccaggacaccggcagccaggccgtgggcttccagcccgaggagcgcatggtggtgcgcggcaaggccgcctacatcgacggcatcggccccgccaccccctacgccggcatgtacggcgagtgggagatcgccaaccccgactgcgtgaacaagggcctgcacgccaccagccgcgagcgcaccatcatcaccctgtgggccgtggtgtgcctgtgccaggccctgttcatgatgacccgcgcccccaaggtgacctgggaggccgtgtacctgcccctggtggagtgcatcctgtactgctgcaccggcgccggctacggcgtgctgcgcatgggcgacggccgcatgctgcccctgagccgcatgtgcagctggctgatcacctgccccatcatgctgagccaggccttcggcatccacgacatgaagctgtacggcatccagctgcgcatgcccatctgcgccgccagcctgatccgcaccgtgtgcggcctgctggccagcaccaccaccgacaccaagatcgccaagtggctgttcttcttcctgggcttcagcttctacaccatggagatcaccaccgtgtggttcatcttccagcagggcatcgccaagttcacccccaccaccgacgtgcccgagaacgccaccgccctgcgccgcctgtacttcatgcgcttcatcttcttcaccagctggaccgccttccccgtgttctgggtgctgagcagcaccggcctgtgcgtgatcagcgagcagaccagcgccctgggctacctgtgcgccgacgccctgtgcaagaacctgtacggcgtgatccagaccaacaccgtgtggggcctgctgggcggcacctggacccccaagaagcaccccaacaaccacgacgaggaggcccccgccaacagccacaaccacaaggaggaggacaccttcgccgccccccacggccgcttcaacagcgcc

SEQ ID NO: 12 - DNA, 987 bp, Artificial Sequence
  cDNA synthetic humanized construct C1ACR_887-2
atggagcaccacagctacgacggcgccatgcgcagcctgcaggccatcagcggcagcttcgccgacatcgagggccactggaacggccccaccagcgtgctggacaaccccgccgtgatcgacggccagctgtacggcgcctacgtgttcatgcaccccgagtgccaccagggcgagtacgcccccatcgtgaagatcatccagctgtgctgggccgtgatctgcatgtgccaggtgctgttcatgttcagccgcgccccccgcgtgccctgggaggccatgtacctgcccggcgtggagtgcgtgatctacaccctggccttcaccggcaacggctacgtgcgcatggtggacggccgcatcctgacctggagccgcctggccagctggctggtgtgctgccccgtgatgctgggccagatcagcgccatcaccctgatcaagtaccagagcatccccctgaaccccatgatgatcgcctgcagcatcatccgcaccgtgttcggcgtgagcgccggcgtggtgcaggaggagcccctgaagtgggtgttcttctggctgggcatcttcttcttcgccttcgagctgctgggctgctacctgatcttcgccatcgccatcgccgacttcgaggcctgcaagaccgagctggccaacaaggtgatcggccgcatcaagaccatgcgcgccatcatcttcatctgctggcccgccatcaccgtgacctgggtgctgagcagcgccggcacctgcctggtggacgacaactacaccaccatcggcttcgtggtgctggacgccgtgtgcaagaacatgtacggcgtggtgatctggagcaccacctggggcctgctgaacggcaagtgggaccgcgactacgcccgcaccttcggccaggaggaggagcagcaggaggaggcccccgacttcgacgccaagggcggcgccatgcccggcgccaacgacaacgtggaggtgaagatgttc

SEQ ID NO: 13 - DNA, 903 bp, Artificial Sequence
  cDNA synthetic humanized construct C1ACR_023-2
atgaccgtgctggaggtgaacgaggccgtgatcatcgacggcgtgatgtacggcgagtacatcttcagccacccccagtgcatccccgccgagctggtgcccgtggtgaaggtgttccagatctgctgggccgtggtgagcatgtgccagagcgccttcatgatgacccgcatgcccaaggtgccctgggaggccatctacctgcccatggtggaggccatcgtgtacgtgatggcctacaccggcaacggctacctgaagatgggcagcggccgcgtgctgccctgggcccgcatggccagctggctggtgacctgccccgtgatgcacggccagatcagcggcatgaccctgatcaagtaccagagcatccccatcaaccccatggtgatcgccgccagcatcatccgcgtggtgatgggcgtgaccgccaccatcgtggtgaccgaggacctgaagtgggccttcttcggctgcggcctggtgtgcctgctgttcgagtacgccagcgtgtacaccatcttcgccgtgaccatcggcgacttcgccgacgccaagaccgacctgggcaaccaggtggtggcccgcctgaagagcatccgcagcatcttcttcgtggcctggaccgccttccccatcctgtggctgctgagcagcaccggcacctgcgtgatcgacgagaacatcagcgccgtggcctacctgatcagcgacgccttctgcaagaacttcaacggcatcctgctgtggagcaccacctggggcctgctgaacggcaagtgggaccgcgacttcgtgcgcgccccccccgccgccgagagcgagaaggacgtgcaggacgccccccgccccaagagcgacgtggaggtgaagatgttcggcaaggtgatcggcagcgtgcgc

SEQ ID NO: 14 - DNA, 909 bp, Artificial Sequence
  cDNA synthetic humanized construct R1ACR_799-2
atggccggcgtgcaggacgactggagcgccgtgagcgtgcccgccttcggccagtacggcacctgggaggccgccaaccccgagtgcgtggagatgggcctgagcaccagcaagaacaccaagatgatccagctgctgttcgccagcatctgcgcctgccaggtgctgttcatggccagccgcgccccccgcgtgacctgggaggccgtgtacctgcccctggtggagaccatcctgtacggcatggccagcaccggcaacggcagcatgcgcctggccgacggccgcatggtgcccatcgcccgcttctgcagctggatcatcacctgccccatcatgctgttccaggtggtgggcatccacgaggtgaaggtgtacggcatcagctgcaagaacatggtgatggccgccagcctggtgcgcaccgtgtacggcatcggcgccagcgtgaccatcgacgacaagatgcgctggatccagtacatgctgagctgcttcaccttcctgttcgagctgtggtgcgtgtaccagatctacaacgccggcctgaagaagttcagcgccgtgaagacccccctgaacaacatcgtgcacagccgcatccagttcctgcgcttcctgttctacctgagctggagcagcttccccatcgtgtgggtgctgagcagcaccggcctgtgcctgatcaacgaggacggcagcgtgctggcctacctggtggccgacatcctgtgcaagaacacctacggcatcatcaacgccaacaccctgttccgcgtgctggacggcaagtggacccccgagaccctggaggaggccgccaacacctacggcatcatcaacgccaacaccctgttccgcgtgctgaacggcaagtgggagcccgagaccgaggaggaggccaagaac

SEQ ID NO: 15 - DNA, 882 bp, Artificial Sequence
  cDNA synthetic humanized construct R1ACR_367-2
atgagctacgtggaggaccccgccttcggcttctacggcagctgggaggccgccaaccaggactgcatcgagcgcggcctgagcaccaccaacaacacccgcatgatccagctgctgttcgccagcatctgcgcctgccaggtgctgttcatggccagccgcgccccccgcgtgacctgggaggccgtgtacctgcccgtggtggagaccgtgctgtacggcatggccagcaccggcaacggcagcatgcgcctggccgacggccgcatcgtgcccatcgcccgcttctgcagctggctgatcacctgccccatcatgctgttccaggtggtgggcatccacgagctgaagtggcgcaccatcagctgcaagaacatggtgatggccgccagcctgatccgcaccgtgttcggcatcggcgccagcgtgaccatcacccccaccatgcgctgggtgcagtacttcctgagcctgagcttcttcctgttcgagctgatcgtggtgtaccagatcttcagcgccggcctgaagaagttcggcgccgtgaagaccagcctgaacaacatcgtgtacagccgcatcatgttcctgcgcttcctgttcttcctgagctggagcagcttccccatcatctggctgctgagcagcaccggcttctgcctggtgaacgaggacgtgagcgtgctggcctacctggtggccgacatgatctgcaagaacctgtacggcatcaccaacgccaacaccctgttccgcgtgctgaacggcaagtggacccccgagaccgaggaggaggccaagcagcccaaggacaacgagcacgcccacgtgggcgagacctacttcgagcagatgctgagcggcctgcgcggcagccgccac

SEQ ID NO: 16 - DNA, 978 bp, Artificial Sequence
  cDNA synthetic humanized construct GcACR_145-2
atgctggacgcccaggacaccggcagccaggccgtgggcttccagcccgaggagagaatggtggtgagaggcaaggccgcctacatcgatggcatcggccccgccaccccctacgccggcatgtacggcgagtgggagatcgccaaccccgattgcgtgaacaagggcctgcacgccaccagcagagagagaaccatcatcaccctgtgggccgtggtgtgcctgtgccaggccctgttcatgatgaccagagcccccaaggtgacctgggaggccgtgtacctgcccctggtggagtgcatcctgtactgctgcaccggcgccggctacggcgtgctgagaatgggcgacggcagaatgctgcccctgagcagaatgtgcagctggctgatcacctgccccatcatgctgagccaggccttcggcatccacgacatgaagctgtacggcatccagctgagaatgcccatctgcgccgccagcctgatcagaaccgtgtgcggcctgctggccagcaccaccaccgacaccaagatcgccaagtggctgttcttcttcctgggcttcagcttctacaccatggagatcaccaccgtgtggttcatcttccagcagggcatcgccaagttcacccccaccaccgacgtgcccgagaacgccaccgccctgagaagactgtacttcatgagattcatcttcttcaccagctggaccgccttccccgtgttctgggtgctgagcagcaccggcctgtgcgtgatcagcgagcagaccagcgccctgggctacctgtgcgccgacgccctgtgcaagaacctgtacggcgtgatccagaccaacaccgtgtggggcctgctgggcggcacctggacccccaagaagcaccccaacaaccacgacgaggaggcccccgccaacagccacaaccacaaggaggaggacaccttcgccgccccccacggcagattcaacagcgcc

SEQ ID NO: 17 - DNA, 987 bp, Artificial Sequence
  cDNA synthetic humanized construct C1ACR_887-3
atggagcaccacagctacgatggcgccatgagaagcctgcaggccatcagcggcagcttcgccgacatcgagggccactggaacggccccaccagcgtgctggataaccccgccgtgatcgacggccagctgtacggcgcctacgtgttcatgcaccccgagtgccaccagggcgagtacgcccccatcgtgaagatcatccagctgtgctgggccgtgatctgcatgtgccaggtgctgttcatgttcagcagagcccccagagtgccctgggaggccatgtacctgcccggcgtggagtgcgtgatctacaccctggccttcaccggcaacggctacgtgagaatggtggacggcagaatcctgacctggagcagactggccagctggctggtgtgctgccccgtgatgctgggccagatcagcgccatcaccctgatcaagtaccagagcatccccctgaaccccatgatgatcgcctgcagcatcatcagaaccgtgttcggcgtgagcgccggcgtggtgcaggaggagcccctgaagtgggtgttcttctggctgggcatcttcttcttcgccttcgagctgctgggctgctacctgatcttcgccatcgccatcgccgacttcgaggcctgcaagaccgagctggccaacaaggtgatcggcagaatcaagaccatgagagccatcatcttcatctgctggcccgccatcaccgtgacctgggtgctgagcagcgccggcacctgcctggtggacgacaactacaccaccatcggcttcgtggtgctggacgccgtgtgcaagaacatgtacggcgtggtgatctggagcaccacctggggcctgctgaacggcaagtgggacagagactacgccagaaccttcggccaggaggaggagcagcaggaggaggcccccgacttcgacgccaagggcggcgccatgcccggcgccaacgacaacgtggaggtgaagatgttc

SEQ ID NO: 18 - DNA, 903 bp, Artificial Sequence
  cDNA synthetic humanized construct C1ACR_023-3
atgaccgtgctggaggtgaacgaggccgtgatcatcgacggcgtgatgtacggcgagtacatcttcagccacccccagtgcatccccgccgagctggtgcccgtggtgaaggtgttccagatctgctgggccgtggtgagcatgtgccagagcgccttcatgatgaccagaatgcccaaggtgccctgggaggccatctacctgcccatggtggaggccatcgtgtacgtgatggcctacaccggcaacggctacctgaagatgggcagcggcagagtgctgccctgggccagaatggccagctggctggtgacctgccccgtgatgcacggccagatcagcggcatgaccctgatcaagtaccagagcatccccatcaaccccatggtgatcgccgccagcatcatcagagtggtgatgggcgtgaccgccaccatcgtggtgaccgaggacctgaagtgggccttcttcggctgcggcctggtgtgcctgctgttcgagtacgccagcgtgtacaccatcttcgccgtgaccatcggcgacttcgccgacgccaagaccgatctgggcaaccaggtggtggccagactgaagagcatcagaagcatcttcttcgtggcctggaccgccttccccatcctgtggctgctgagcagcaccggcacctgcgtgatcgacgagaacatcagcgccgtggcctacctgatcagcgacgccttctgcaagaacttcaacggcatcctgctgtggagcaccacctggggcctgctgaacggcaagtgggatagagacttcgtgagagccccccccgccgccgagagcgagaaggacgtgcaggacgcccccagacccaagagcgacgtggaggtgaagatgttcggcaaggtgatcggcagcgtgaga

SEQ ID NO: 19 - DNA, 909 bp, Artificial Sequence
  cDNA synthetic humanized construct R1ACR_799-3
atggccggcgtgcaggatgactggagcgccgtgagcgtgcccgccttcggccagtacggcacctgggaggccgccaaccccgagtgcgtggagatgggcctgagcaccagcaagaacaccaagatgatccagctgctgttcgccagcatctgcgcctgccaggtgctgttcatggccagcagagcccccagagtgacctgggaggccgtgtacctgcccctggtggagaccatcctgtacggcatggccagcaccggcaacggcagcatgagactggccgacggcagaatggtgcccatcgccagattctgcagctggatcatcacctgccccatcatgctgttccaggtggtgggcatccacgaggtgaaggtgtacggcatcagctgcaagaacatggtgatggccgccagcctggtgagaaccgtgtacggcatcggcgccagcgtgaccatcgacgacaagatgagatggatccagtacatgctgagctgcttcaccttcctgttcgagctgtggtgcgtgtaccagatctacaacgccggcctgaagaagttcagcgccgtgaagacccccctgaacaacatcgtgcacagcagaatccagttcctgagattcctgttctacctgagctggagcagcttccccatcgtgtgggtgctgagcagcaccggcctgtgcctgatcaacgaggacggcagcgtgctggcctacctggtggccgatatcctgtgcaagaacacctacggcatcatcaacgccaacaccctgttcagagtgctggatggcaagtggacccccgagaccctggaggaggccgccaacacctacggcatcatcaacgccaacaccctgttcagagtgctgaacggcaagtgggagcccgagaccgaggaggaggccaagaac

SEQ ID NO: 20 - DNA, 882 bp, Artificial Sequence
  cDNA synthetic humanized construct R1ACR_367-3
atgagctacgtggaggaccccgccttcggcttctacggcagctgggaggccgccaaccaggactgcatcgagagaggcctgagcaccaccaacaacaccagaatgatccagctgctgttcgccagcatctgcgcctgccaggtgctgttcatggccagcagagcccccagagtgacctgggaggccgtgtacctgcccgtggtggagaccgtgctgtacggcatggccagcaccggcaacggcagcatgagactggccgacggcagaatcgtgcccatcgccagattctgcagctggctgatcacctgccccatcatgctgttccaggtggtgggcatccacgagctgaagtggagaaccatcagctgcaagaacatggtgatggccgccagcctgatcagaaccgtgttcggcatcggcgccagcgtgaccatcacccccaccatgagatgggtgcagtacttcctgagcctgagcttcttcctgttcgagctgatcgtggtgtaccagatcttcagcgccggcctgaagaagttcggcgccgtgaagaccagcctgaacaacatcgtgtacagcagaatcatgttcctgagattcctgttcttcctgagctggagcagcttccccatcatctggctgctgagcagcaccggcttctgcctggtgaacgaggacgtgagcgtgctggcctacctggtggccgacatgatctgcaagaacctgtacggcatcaccaacgccaacaccctgttcagagtgctgaacggcaagtggacccccgagaccgaggaggaggccaagcagcccaaggacaacgagcacgcccacgtgggcgagacctacttcgagcagatgctgagcggcctgagaggcagcagacac

SEQ ID NO: 21 - DNA, 948 bp, Artificial Sequence
  cDNA synthetic humanized construct GcACR_457-1
atgtccgcctgcccccccaggacacccagatacggcaacatgtccgctgccgacaagagggccgagtacctgcagaaggtcatcgacctgagccctgagatctatggcagctacgccagggacaacgccgagtgcctcgccatgagcgtggtcaccatgcctttcgtgaagatcacccagctgtgtatggccgtgatgagcgcctgccaggtcctgttcatgatcagcagagcccccagagtgccctgggaggccatctatctccccgccaccgagatgatcagctacagcctggccttcttcggccagggatatgtgaggctggccaatggcaaagtgctgccctggtgcagaatggccgcctggctgtgcacctgtcccattatgctgggcctggtgtccaacatggccctcatcaagtacaagagccagcccctgaaccccatgatgatcgccgcctccatcatcaggacagtgttcggcatctccgccaccatggccgaaaccaacgacgtgatctggacccactttttcttcgccttcgtgtgcttcattttcgagatgtcctgcgccttcgccatcttcgccctgaccatcgacgacttccagcagattggcagccctctggccatgaaagtggtgaagaggctgtacctgctgaggatggtgttcttcggcacctggagctgctttcccatcctgtggatcatcagcagcacctacatgtgcgtgatcgacgagaacgctagcgctctgctctacctggtggccgatatctcctgcaagaactgctacggcctgctgctgtggagcaccacctggggcctgctgaacggcaagtgggacagagagtatgccaggaacagggacgaagccggcatgctcatggagaccgatgagaagcagggcgtgatcgagcctcccaaagagaacttcgacgtgaagctg

SEQ ID NO: 22 - DNA, 1020 bp, Artificial Sequence
  cDNA synthetic humanized construct GcACR_439-1
atggccgagaccggcgtgatcagaagccaggaagactactacaggctcgtggagaacagggagacaatcggcacatactgggattcctccctggtgtacggcgagtgggccaggaacaacggcaactgcctcagacagtccgaggtggacgtgcagttcgtgaagatcctccagctctgtatgagcgtggtcagcagctgccaggtgctgttcatggtgagcagagctcctctggtgccctgggaggccatctacctccccatgaccgagagcattacctatggcctggccttcaccggcaacggctacatcagactggctacaggcaagatcctgccctgggccaggatggccagctggctgtgcacctgccccatcatgctgggcatggtgtccaatatggccctggtcaagtacaagagccagcccctgaaccccctgatggtggccagctccatcatcagggtggtgttcggcatctccgccacaatgagcgacgacagccagcccgccaaatgggtgttcttcttcatcgccgtgattttcttcctcttcgagctgacctgcgtgtacgctatcttcggaatgaccatcgccgactttgcccaaatccagtcccccctgggcgatgccgtggtctacaggctgaacatcctgaggggcatctttttcgccagctggacatgctttcccatcctgtggctggtgtcctccaccagcgcctgcctgctggacgagaatatcagcgccgtgctgtacctgatggccgacgccctctgtaagaacagctacggcattgtcctgtggagcacaacctggggcctgctgaacggcaagtgggacagagagtatcccagaaacagggacatcgacggcctgatgatggacatcgaccccgccaaggagaagcaactggaggagatgcccaaccagaacttcgacatcaagatcatgggccagaccatcgcttccgtgaagagaagctgcagaaggcctagagccgatatggagagccaggat

SEQ ID NO: 23 - DNA, 1053 bp, Artificial Sequence
  cDNA synthetic humanized construct GcACR_203-1
atggccgctaggctggccaaggtggtgcacctggccaggctggtggtgctgttcgccgtgatgctggacgctcaggataccggaagccaggccgtgggctttcagcccgaggagagaatggtcgtgagaggcaaagccgcctacatcgacggcatcggccctgccacaccttatgccggcatgtatggagagtgggagatcgccaatcctgattgcgtgaacaagggcctgcacgccaccagcagggagagaaccatcattaccctgtgggccgtggtgtgcctctgtcaggctctcttcatgatgacaagggcccctaaggtgacctgggaggccgtgtacctgcctctggtggagtgtatcctgtactgctgcacaggagccggatacggcgtgctgaggatgggcgatggcagaatgctgcccctgagcaggatgtgctcctggctgatcacctgccccatcatgctctcccaggcctttggcatccacgacatgaagctgtacggaatccagctgaggatgcccatttgcgccgcctccctcatcaggacagtgtgcggcctgctggccagcacaaccaccgataccaagatcgccaagtggctgttcttcttcctgggctttagcttttacaccatggagatcaccaccgtgtggttcatcttccagcagggcatcgccaagtttacccccaccaccgatgtgcctgagaacgccaccgccctgagaaggctgtacttcatgaggttcattttttttaccagctggaccgccttccccgtgttttgggtgctgagcagcaccggactgtgcgtcatcagcgaacagacaagcgctctcggctacctgtgcgccgacgctctgtgcaagaacctgtacggagtgatccagaccaacaccgtgtggggcctgctgggaggaacctggacccccaagaagcaccccaacaatcacgatgaggaggctcccgccaacagccacaaccacaaggaggaagacaccttcgccgcccctcacggcaggttcaactccgccaaagagatcgaggat

SEQ ID NO: 24 - DNA, 873 bp, Artificial Sequence
  cDNA synthetic humanized construct GcACR_243-1
atgattatcaccctgtgggccgtggtgtgcatgtgccaggccctgttcatgatgaccagggcccccaaggtgacatgggaggccgtgtatctgcctctggtggagtgcatcctctactgctgcaccggcgccggatatggccagctgaggatgggcgatggcaggatgctgcccctgagcagaatgtgcagctggctgatcacctgtcctatcatgctgtcccaggcctttggaatccacgacctgaagctgtacggcatccagatgaggatgcccattctggccgccagcctcatcagaagcgtctgtggcctgctggcctccaccaccaccgacaccaaggtggtgaagtggtttttcttctttctgggcttctgcttcttctccatggagatcggctccgtgtggttcatcttccagcacggaatcgccaagttcgcccctacaaccgacgtgcccgagaatgccacagctctgaacaggctgtacatcatgagggtgatcttcttcagcagctggaccgcctttcccctgttctgggtgctgtccagcaccggcctgtgcgtgatcagcgagcagacaagcgccctgggctacctgtgcgccgatgccttctgcaagaacctgtacggcgtgatccagaccaataccgtgtggggcgtgctgggaggcacatggacccccaagaaacccccctccaaagatgacgaggaggcccccaccaactcccacaacgacaaggagatggacgccttcaccgccccccatggcaggttcaattccgccaaagagatccaggacacctacaatagcgacacctacttcatcagggccgccaaggccgtgagcgtgaggatgtccagaaagaagggc

SEQ ID NO: 25 - DNA, 933 bp, Artificial Sequence
  cDNA synthetic humanized construct ZipACR/PsuACR_973-1
atggccgccctccatcaggctctggaggagctgggacagggagccagcaggcacctcctgtacagagcccccgaggagatggtgtatggcaagtgggtggaggccaaccctaggtgcatcgaactgcacgaccactccgactacgtgtacgtgttccagctgtgcttcgccgtggtgtgcgcctgccaggtgatcttcatgttcaccagggcccccaacgtgggctgggaagccatctatctgcccctggccgaggtcgtgacctacagcattgccgccaacggagagggcgtgctgaggatggccgacggcaggtacttcaatttcgccaaactggccggatgggccgtgtgctgccccattatgctcatccagatcggaggcatggcccagatcaagtacaggaccatccccctgaacaacgtggtgctggccgctagcctgaataggatcatcttcggcatggccagcgccatcacagcctccgaccctgctagatggggcttttacttctgcgcctggatctgttacctgaccgaggtcggcatcaccctgaccatcatggccgtggccatcagcgacttcagcaagatcaagacagagctcggccagtgggtggtgggccgtattcaaaccatgaggatcatctttctggtcgcctggaccagcttccccgtcgtgtgggtgctgggctacaccggattctgcgtgatccacgaggactacatcgccctgctgtatctgttcgccgacctgctctccaagaacacctggggagtgatgatgtggcataccacctgggtgaaactcaacggcaaatgggacagggaatttgccgccgccggaggacacgaggctctgaagaaggccctggagcaggacgtggaaatcggcgccggcgagaagaaccagaaccagctgaccgccaggcag

SEQ ID NO: 26 - DNA, 903 bp, Artificial Sequence
  cDNA synthetic humanized construct PsuACR_433-1
atggctgagctggtggacgagtacggcctgtgggccctggagaaccccgagtgcatggagatgtcctacaccagccccaacaccaaaaccgcccagctgtgcttctgcatggtgtgcctgtgccagatcatctggatcatgggacagtggcccaaagtgggatgggaaagcatctacctgcccgcttgcgagatgacactgtattccctggccgcttccggcaatggcttcatccagctggccgacggcaggatcatcccttatgccaggatggccgcctggatttgtacctgtcccgctctgctgatgcagatcaacggcctggccaagtgcaagctgggccacttcaacctgaacaccttccaggtggtggccgatctgatcatgaacgtgatgggactgaacgccacactcaccaaccacacctacctgaagtggatctacttcggcattgccatggtgctcttcggatgcatcatgggcatcacccacgatatcttctcccagaccgctaggaagtggaagtccgtggacacacccctggcccagaaggtcgcctggagggtcaccttcctgaagtacctgttcagcctcagctggagcatctaccctctgctgtggattttatccccccaggccacctgtagcctgagagaagacgtgatctcctgcctgcacttcgtgtccgacttcttcgccaagaacatctttggcctggtgatgtggtggaccctctggagagacctggacggcgactggagcttcgagcaccactacccccccgatcaggagcaggctaggagctccgctagcgccacacactccctccccggcaagagcattggcctggccgagagcatcaacttcgtgaaggacacccaggactgccacgatcacgatggc

SEQ ID NO: 27 - DNA, 891 bp, Artificial Sequence
  cDNA synthetic humanized construct R1ACR_477-1
atggccctgtacacctcccagtttggcatcgcctacggcagctgggaggccgagaacgacgattgcctgagcatgggcatcgctagcggagcccccgtgaagatcatccagctgtgttgggccgtggtgagcctgtgccaggtgattttcatgctcacaagggcccccagggtggcttgggaagccatctacctgcccctgtgcgagaccatcacctacttcctggccttcagcggcaacggctacctgaggatggccgacggccagatcttcccttgggccaggatggccagctggctggtgacctgccctgtgatgctgggccaggtgagcgccatggccctgatcaagtacaaaagcctgcccctgaacaatatgatggtggccgcctccctgatcagggtggtgatgggcatctccgccaccatcacacccaacgagcagctgaagtggatgttcttctttttcggcgtgagcatgctgacctttgagtatttctgcgtgttcaccatcttcagcatcaccatcgccgacttccaggagatcgacacacccaggtccaggttcgtggtcggcaggctgaagatcctcaggattgtgttccactccgcttggacagccttccctatcctgtgggtggtgtccagcaccggcctgtgcgtgctcagcgagaatatctccagcgtgtgctacatgctcgccgacgccgcttgtaaaaatggctacggactggtcctgtggaacaccacctggggccacctgaacggcaagtgggatagagagttcgccaagaatgccgacaaggacggagagatgaaggccatcgaggaaggagctgctggacccgccgctgacgccaagaaggacgaggatatcaagctgttcggcaag

SEQ ID NO: 28 - DNA, 825 bp, Artificial Sequence
  cDNA synthetic humanized construct RsACR_995-1
atgggcatcgccagcggcgcccccgtgaagattatccagctgtgctgggccgtggtcagcctgtgccaggtgatcttcatgctgaccagggcccctagggtggcttgggaggccatctatctgcccctgtgcgagacaatcacctacttcctggccttcaccggcaacggctacctcaggatggccgacggccagatcttcccctgggccaggatggcctcctggctggtgacctgccctgtgatgctgggccaggtgtccgccatggccctgatcaagtacaagagcctgcccctgaacaacatgatggtggctgcctccctgattagggtggtgatgggcatcagcgccacaatcacccccaacgagcagctgaagtggatgtttttcttcttcggcgtctccatgctgaccttcgagtacttctgcgtcttcaccatcttctccatcaccatcgccgacttccaggagatcgacacccccaggagcaggctggtggtgggaaggctgaagatcctgagaatcgtgttccacagcgcctggaccgccttccccatcctctgggtggtgagcagcaccggcctgtgtgtgctgagcgagaacatcagctccgtgtgctacatgctggccgatgccgcctgcaagaacggctacggcctgatcctgtggaacacaacatggggccatctgaacggcaagtgggacagggagttcgccaagaacgccgacaaggatggcgagatgaaagctatcgaggagggcacagccggacctgccgccgacgccaagaaggacgaggacatcaagctgtttggcaagcccgtg

SEQ ID NO: 29 - DNA, 900 bp, Artificial Sequence
  cDNA synthetic humanized construct R1ACR_877-1
atgagcgccaccgaaaaccctgccttcggcctgtacggcacctgggagaacgaccacccccagtgtgtggagatgggcctggacaccagcagccagacaaacatgatccacctcctgttcgccagcatctgcttctgtcaggtgctctttatggcctccagagctcccagagtgacctgggaggccgtctacctgcccctggtggagaccgtgctgtacggaatggccgccagcggcaacggcaacctgaggatctccaacggcaggatggtgcccatggccaggttctgcagctggctgatcacctgtcctatcatgctgctgcaggtggtggtgatgcacgacgtggtgtggttcaacgtgagcaccaggtacatggtgctggccgccagcctgatcaggaccgtgttcggcatcggcgccagcgtgaccaccaacgataccatgagatgggtgcagttcctgctgtcctgcatctgcttcgccttcgagatggtggtggcccaccagacctacaagggagccctgaagaagtttgccgccgtgaagaccaccctgaacggcgtggtctacgccaggatcatgtttctgaggttcctgttcttcttcagctggaacgccttccctatcatctgggccctgagcagcacaggcctgtgtctgatcagcgaggacgccagcgtgctgtcctatctcgtggccgacctgatctgcaagaacatctacggcatcaccaacgccaacaccgtgttcagagtcctggacggagcctgggtccctgagaccgaggaacacgcccaggccaaggaccacctgacctacctcgagatgatgatgaaccacaacctgggcatcggcagacatcatgacgaggagctgcaaatggaagtgggccatgac

SEQ ID NO: 30 - DNA, 900 bp, Artificial Sequence
  cDNA synthetic humanized construct R1ACR_447-1
atggtgtggtcccatgtggacgagcctgccctgggaccctatggcagctgggaagccgccaaccccgattgtgtggccctgggcctgaccacctccaagaacaccaagatgatcctcctgctgttcgccagcatttgcgcctgtcaggtgctgttcatggccagcagggcccctaggattacatgggaggccgtctacctgcctctgatcgagaccatcctgtacgctatggccagcaccggcaatggccagctgaggctgggagacggcagggtgctgcctatcgccaggtactgctcctggctgatcacctgccccatcatgctgttccaggtggtgggactctacgatctgaagctctggggaatctcctgcaagcacatggtgatggccgccgccctgatcagaaccgtctttggaatcggcgcttccgtgacactggatgaccagatgaggtgggtgcagttctttctgtcctacgccttcttcttctttgagctgtactgcgcctacatgatcttcggcgctgccctgaagcagttcggcgaagtgagaaccaccctgaatagcatcgtgtactccaggatcatgctgctgaggttcattttcttcctgagctggaacagcttcggcatcatctggctgctgagcagcaccggcctgtgcatgatttccgaggacgtgtccgtgctggcctacctggccgctgacatgctgtgcaagaacacctacggcatcatcaacgccaacaccctgttcagagtcctggacggcaagtggacccccgagacactggaggaagccgcctccggcgtgaagaaggccaaccccaaccacgtgggcgagacatacctggagaggatgatggccggcattaggggcagcgctaagcccgaggag

SEQ ID NO: 31 - DNA, 891 bp, Artificial Sequence
  cDNA synthetic humanized construct R1ACR_741-1
atgtcctacgtggaagatcctgccttcggcttttacggcagctgggaggccgccaatcaggactgcatcgagagaggcctgtccaccagcaacaataccaggatgatccagctgctgttcgcctccatttgcgcctgccaggtgctgttcatggcctccagggcccctagagtgacctgggaggctgtgtacctgcctgtggtggaaaccgtcctgtacggcatggccagcaccggcaacggcaacatgaggctggctgacggcaggatcgtgcctatcgccaggttctgcagctggctgatcacctgccccatcatgctcttccaagtggtgggcatccacgagctgaagtggagaaccatcagctgcaagaacatggtgatggccgctagcctgatcaggaccgtgttcggaatcggcgccagcgtcaccatcacccccaccatgaggtgggtgcagtacttcctgtccctgagcttcttcctgttcgagctgatcgtggtgtaccagattttctccgccggcctgaagaaattcggcgctgtgaagacctccctgaacaacattgtgtactccaggatcatgttcctgagattcctgttcttcctgagctggagcagcttccccattatctggctcctgtccagcaccggattctgcctggtgaacgaggacgtcagcgtgctggcctatctggtggccgacatgatctgcaagaacctgtatggcatcaccaacgccaacaccctgttcagggtgctgaatggcaagtggacccccgaaaccgaggaagaggccatgctgcccaaagaccacgaacacgcccacgtgggcgagacctacttcgagcagatgctgagcggcctcagaggcagcaggaggagggaccac

SEQ ID NO: 32 - DNA, 840 bp, Artificial Sequence
  cDNA synthetic humanized construct GcACR_197-1
atgcaggcctcccagatcagctcctcttactttggccagtggtctgccacaaacatcgactgcgtggccaaggccgatatcagctccggcgtgcagacctttctgctgtgcttctgtatcatcagcgcctgccaggccctgtacttttcctatcactgtctgccacaggcaggagcacggagaaggagcacatgggagttcatcgccgtgcctgcagtggaggccctgtgctacggcctggcatctagctcctctcaagtgggctacttccggctgttcgacggaaggaccgtggtgtggctgaggacagtgctgtggattctgaccgtgccagtgctgctgctgcagatcggcaagatgagctacgtggtcatccagggcgtggaagtgaattccctgatcatctttgtgggcctgctgatggtggtgtttggcttctctgccagcctgtccaccgtgatgggcatcaagtggatgttctttttctttggcctggtgtgcatggccgtggtgttcctgggcgtgtataagatcatggtggacgccggctcctactatatctccctgaactctcaggatggcctgcagatcgcctctcggatcagaatcctgatgatcatcttctttttctcttggaccgccacacccctgttctgggtgctgagcatcgagggagcatgcgtggtgtctgagagcatcacagtggtgtgctttgccgtgatcgactccctggccaagaatatcttcggcatcatcctgtgggataccgtgtggaacagctccctggagggccggtggacatctaatctgcagatcatggccaccgagatgagcatgctg

SEQ ID NO: 33 - DNA, 864 bp, Artificial Sequence
  cDNA synthetic humanized construct GcACR_201-1
atggaggcagcagacgaggaggatgtgggaggaggcgacctgtggaacgtgaataccggctgcctgtatggaaacctggcatccgcctctccactggtgaggcacttcgagctggcatgggccgtgtgctgtctgacccaggccttctttaagtttacacagtggccaagggcagcatgggagggcgtgtacatccctctgtgcgagacagtgcagtattctctgctgttctttaagctgggctacatccaagtgggagtgaaccagggagcatacctgccatgggtgcggtatagcttctggatgatcaccacacccgtgctgctgaatcagatccagcagatctgcgaggtgaagatctggggcgccagaatgggccctgtgcagacctgggccaacatcgccatggtgacactgggcatctccggcgccctgatccagaatccaaccatgaagtggctgtgcttcgtggcaggcacactgctgggcgccctgatctttctggtggccttcctgatctttcacaaggcctctgagcagttcaaggagatcggccaccccaaggccgacagggtggtgaagtacctgtggtatctgcaggtgtgcttctttagctcctggagcgtgtttcctctgctgtggatcatctccgcccagggcacctgtatcgccaacgaggacatcgtgtctagcatccacatcccagccgatatcctggccaagaacgtgttcggcctgatcatctggcacatccggtttcacctgtgcgacggctactgggatatcgagagattcaagcagaagctgctgcaggacaagctgctggccctgtttgataatcctgaggagctgagacaggatcac

SEQ ID NO: 34 - DNA, 855 bp, Artificial Sequence
  cDNA synthetic humanized construct HpACR_213-1
atgaccgtgatctacggacagtgggcagcacagaacccaggatgcgtgtctatgaccaatatcgacctgggcgtgaggacattcgagctgtgctggggcatcatgtgcgcctgtcaggccgtgttctttgcactgaggtacccaagggcaacctgggagttcgtgatggtgcctctgacagagtgctttatctacggcctgggctattccgagatcggctatatcctgctgtgggacggccggcaggtgctgtggaccagaacagtgctgtggctggccaccgtgcccatcctgctgaaccagatcaatgccatggccgccgtgcacatctacggcgtggatctgaacatcatgcagatgtatttctccacactgatgatctgcctgggacacaccgccgccctgacacacaatcagtctctgaagtggctgttctttatcatggccatggccatcttcggcgtgatctgttttatcaactactataccttcgccgccgccgagaagcactacaaggagagcggctccgagaatgatctgaagatcgccaagcggatcagactgctggccatcatcttctttacctcttggacaatgtatcccctgttctttgtgctgagcgtggagggagcatgcgtggcatctgagagcgtgatcctggtgtgctttgccctggccgacctgctgagcaagaacgtgttcggcgtgctgttttgggacaccctgtggaatatgcaggatggcaagtggacatcctacggcaccgtgacattctttaagaccacagatagcgtgaccaagacagccatctccacagccatggaggaggagtattccaagcagaaggtgtctaga

SEQ ID NO: 35 - DNA, 873 bp, Artificial Sequence
  cDNA synthetic humanized construct HaACR_359-1
atggcaaccccagcacctgcaggagcaccactgaccacaaccccagtgccatacggacagtgggaggcaaacaatcctgcctgcgtggccctgttcacaatgccagcccaggtgcagaccctgatgctgtgctggtgtatcatctgctcttgtcaggccatctttctgatgagcaggtggccaagagtgggatgggagctggtggcagcaccagcaatcgagtccatcgtgtacggcctgggctatcagggcatcggctacgtgaagatggcctctggccagcctctggcctatgccaggttctttatgatgatcacaatcaccccaatgatcctgctggacatcaacaagctggcaagcgtgcgcctgctgggcctgaacatgaattccatgcagctgggcgccaatgtgctgctgtggatgttctggcaggccagcacaatgtccaccctggtgggcctgaagtgggtgttctttgtgctgggcctgctgtgcctgggcctggtgctgggcacatctttcatgatctttgctgccgcccggcagagctttctgctgaagggaacaggagcaggcgactgggtggcacagagaatcacctacctgcagatggccttcctgcccacatggaccgcctatatggtgctgtttgtgctgagcggagagggagcatgtatcgcaggagagggcgtgatcctgcctatcgccatcgtgatggatctgttccagaagaacgtgcacggcctgatcatctggtccaccttctggaactttctgggaggctcttggaatccagagcggctggcagaggagagggcaccttccaggatcgcaggcctgaaagtgatcgagaacaatgcccaccctgtggat

SEQ ID NO: 36 - DNA, 900 bp, Artificial Sequence
  cDNA synthetic humanized construct HrACR_495-1
atggcaacaccaggacctgcagcagtgccatccatgaccacaaccccagcaccatacggaacctgggaggcaaacaatccatcttgcgtggccctgttcaacagctccccccagatccagacactgatgctgtgctggtgtatcatctgcacctgtcagtctatctttctgatgagcaggtggcctagagtgggatgggagctggtggcagcaccagcagtggagtgtatcgtgtacggcctgggctatcagggcatcggctacgtgaagatggcctctggccagcagctggcctatgccaggttctttatgatgatcacagtgaccctgatgatcctgatcgacgtgaacaagctggcctctgtgcgcctgctgggcctgaacatgaatagcatgcagctggccgccaatctgctgctgtggatgttctggcaggcctctacaatgagcaccctgtccggcctgaagtgggtgctgtttgtgctgggcctgatctgcctgggcatcgtgctgggcacatccttcatgatcttttctgccgcccggcagggcttcctggacaagaataccggagcaggcgattgggtggcacagagaatcgtgtttctggaggtggtgttctttcctacatgggccggctacatggtgctgtatgtgatcagcggagagggagcatgtatcgcacctgagacagtgatcatcccagtggccatcgtgctggatctgttccagaagaacgtgcacggcctgatcatctggagcacattctggaactttctgggcggctcctggaatccaggccggctggcagaggagagggcacctagccgcctggccggcctgaaagtgatcgagaataccgcccacggcttcgacgatgaggccctgtccgagggcttt

SEQ ID NO: 37 - DNA, 900 bp, Artificial Sequence
  cDNA synthetic humanized construct PsuACR_353-1
atggaggtggagatggagatggagatggagggcctgggcaacagcacatccatgttcgaggaggtggtgggatgcgatctgggagcaagcccagtgggcgagacagagatgcacgtggtgcagtggatcggcagcttcatctgcatctttcagtgtctgtggttcctgtccaagtggcctaactttacctgggacacaccatacatccctgcagtggaggcagtgggatatggcctgatggccaatggcttcggctacgccgagctgtttgagggcgagcggcacgtgccttggctgagatattccatctgggtgctgtctacaccagtgctgctgtgggacatccaggatatcctggacgccttctacaggggaaccagactggcacctgtgcagtctctggccaatctgtttctgctgtgcttcggctttatcggcgccatcgagcacgtgctggccgtgaagtacctgtgctatagcatcggcgtgatgatgctgggcgtggtgttcttttgtgcctataacatcgtgggccaggccgtggccaatttcgaggccaccgatacagcacagggcaggctggtggcctttcgcctgaagctgatcgcctgcatcttcatgacatcttttagcggctacccaatcctgtggattctgtccaaccagggcctgtgcctggtgccagagatgatcatcgaggtggtgcacatccccctggacctgatcgccaagaacctgtttaccctgctgacatggcacacccactatatcctgctgaatggcgattggtcttgcgaggcaatggtggcaaggctgcaggagcagaatgacatcgagatggcacagcagccaagggaggcaggacagctgcgcaaggatctgatgcagaaggcccagggc

SEQ ID NO: 38 - DNA, 918 bp, Artificial Sequence
  cDNA synthetic humanized construct RaACR_687-1
atgggcgacatgtcccacatcgagacagtgtactatgtgaacggaaccgaggtggacccctggacacacaggtgccagctgggagtgcctggaggctcccagggcatccacaccttccagctgacatggagcctgatctgtaccgcccaggccatctttctgatgacaaagtggccaaaagtgggctgggaggccatctacatgccctgctgtgaggccctgtcttatggcctgctgagcgccggacacggatacctgaagctgcacggcgagtggcactacatcccttggatcagatatgccgcctggatggtgaccacaccagtgctgctgaatcaggtgcagggcgtgctggacgtgaaagtgggccacctgagactggccccaatccagagcctggccaacctgatcatgatcgtgatgggcgtgtccgcccacctgcaggagaatatcgcctggaagtgggtgttctttctgatcggctgcgtgatggccctggtcatctactgggtggcctggctggtgttctctacccagatcaagtactatgccgagacaaagacagccgtgggcgatagggtggtgtttcagctgaagctgatcgccgtggccttctacgtgggctggtctgcctttcccctgatctggatgctgagctccgagggcctgtgcgcaatcgaggagcacatcgctgccgccgcccacatccctgcagacgtgctgagcaagaacatcttcggcctgctgatgtggcacacaaggtggatcgtgctgggcggctattgggatgtgaataagtttaaggagacaatcgcccagcagaagctggaggaggccaagggcgaggagacaccccaggagaagaggaaggccctgatgggacaggcagaggcctgcttcaagatggccgacgcctgt

SEQ ID NO: 39 - DNA, 936 bp, Artificial Sequence
  cDNA synthetic humanized construct R1ACR_497-1
atggagtaccagagcctggacgattttccactggagacactgtacttccggaacggcacagaggtggacccctatgatcacagatgcaagctgggcatccctggaggctcccagggcatccacacctttcagctggtgtgggcactgctgtgcgcagcacaggccctgtttctgttctccaagtggccaaaagtgggctgggaggccatgtacatgccctgctgtgaggccctgtcttatggcctgctgagctccggctacggctatatgaagctgcacggcgagttccactacctgccttggatcaggtacgccgcctggatcgtgaccacaccagtgctgctgaaccaggtgcagggcatgatcgacatcaagatcggctctctgaggctggcaccaatccagagcctggcaaacctgatcatgatcatcatgggcgtgagcgcccacctgcaggagaatcacgcctggaagtggctgttctttttcgtgggctgcgtgatgtgcgccgtgatctactatatcgcctggctgcagtttgccaaagtgaatgagctgtacgccgccaccaagacagagatcggcgataaggtgtgctttcagctgaagctgatcatgttttgtttctatgtgggctggtccatgttctctgtgatctggctgctgtctagcgagggcctgtgcctgatcgaggagcacatcagcgccgccgcccacatcccaggcgacatcctgagcaagaactcctttggcctgctgatgtggtataccaggtggatcacactgggaggagcatgggatatcaataagttcctggagaagcaggccaaggagaaggccgaggccgtgctgacacctgccgagaagaggaaggccatcatgaagcaggcagaggcatgcttctccatggcagacgcctgtctgcagcaggccaat

SEQ ID NO: 40 - DNA, 921 bp, Artificial Sequence
  cDNA synthetic humanized construct RsACR_665-1
atggactacaccagcctggacgattttccactggagacactgtacttccggaacggcacagaggtggacccctatgatcacagatgcaagctgggcatccctggaggctcccagggcatccacacatttcagctggtgtgggcactgctgtgcgcagcacaggccctgtttctgttctccaagtggccaaaagtgggctgggaggccatgtacatgccctgctgtgaggccctgtcttatggcctgctgagctccggctacggctatatgaagctgcacggcgagttccactacctgccttggatcaggtacgccgcctggctggtgaccacaccagtgctgctgaaccaggtgcagggcatgatcgacatcaagatcggctctctgaggctggcaccaatccagagcctggcaaacctgatcatgatcatcatgggcgtgagcgcccacctgcaggagaatcacgcctggaagtggctgttctttttcgtgggctgcgtgatgtgcgccgtgatctactatatcgcctggctgcagtttgccaaagtgaatgagctgtacgccgccaccaagacagagatcggcgataaggtgtgctttcagctgaagctgatcatgttttgtttctatgtgggctggtccatgttctctgtgatctggctgctgtctagcgagggcctgtgcctgatcgaggagcacatcagcgccgccgcccacatcccaggcgacatcctgagcaagaactcctttggcctgctgatgtggtataccaggtggatcacactgggaggagcatgggatatcaataagttcctggagaagcaggccaaggagaaggcagaggccgtgctgacccctgccgagaagaggaaggccatcatgaagcaggccgaggcctgcttctccatggccgatgcctgt

SEQ ID NO: 41 - DNA, 855 bp, Artificial Sequence
  cDNA synthetic humanized construct R1ACR_653-1
atgccatacgataactatggctggtgggagcgggagaatgccgactgcatctctaagatggatgccaagctggaccagatgcacctggtgcagctggccatgtccatgctgtgcttttgtcagctggtgttcatgacctctagatttccaaagcaggcatgggaggcaatctacctgccagcagcagagtgcgtgctgtatgcaatggcagcaagcggaaacggatacctgaggctggcaaatggccgcctgctgccatgggcaaggatggcatcttggctggccacctgtcctgtgatgctgaaccagatcaatacaatcagcccactgaagctgtatggcatcaacctgaacaatgtgcagatcgccctgtccgtggtgcgcgtggtgaatggcgtgtctgccagctccgtggacgatgacagcctgaagtggggcttctttttcatgggcgtgttctgcctggtcatcgagttttccatcgcctacaacatcttcgccctgggcatggccaagtttaaggaggccggcaaggattctgacatctgcgcccgggcctgtgatagactgcaggtgctgcggggcatctttttcagcgcctgggccatgttccccatcatctggctgttttcccctcagggaacctgtctgctgggagaggatgtgatccctctgctgtatctgttcgccgacctggtgtgcaagaattgttttggcatcctgtggtacgataccacatggaggctgctggacggcaagggatggaaccccgagctgcagctgacagacaatgaggtggaggagctggtgcacgccaaggaagaggtggaggatcaccaccaccctcacgacgcc

SEQ ID NO: 42 - DNA, 891 bp, Artificial Sequence
  cDNA synthetic humanized construct R1ACR_6367-1
atgtctgccatcacagaccccgccctgggcctgtacggaacctgggaggccgccaacaaggagtgcgtggataagggcgtggagtttagctccaatacccggatgatcgtgctgctgtttgccagcgtgtgcttctgtcagacagtgtatatgaccagcagggcaccaagggtgaactgggagggctgctacctgcctctgtgcgagacatttctgtatggcctggcagcaagcggaaacggaatgatccggctgtccaatggcagaatggtgccaatggccaggttctgctcctggctggtgacctgtcccatcatgctgatgtctgtgctggccatccacgacgtgcgctggatgggcgtgcctgtgaagaatatggtcatcgcagccacactgatcaggaccgtgctgggaatcggagcatctagcaccatcacagagacactgagatgggtgcagtactgcatcgcctgtgccgtgttcgtgttcgagctgacaatggtgaaccagctgtttatcgccggcctgcagaagttcggcgtggtgaagaccaagctgaatgacatggtgtatatgaggatcatgatcctgcgcgccctgttctttttcagctggacagcctttcctgtgatctggattctgtcctctaccggcttctgcgtgatcggagaggacggctccgtgctggcatacctggtggccgacttcctgtgcaagaacgtgtatggcatcatcaacacaaataccctgttccaggtgctggatggcgagtggaagccagagacaagggacaccgatggcaagatcgccacaaatcgccacgcccacatcggcgagacatacctggagatgctgctggcccggatcagacggcacagagagggcgagctg

SEQ ID NO: 43 - DNA, 915 bp, Artificial Sequence
  cDNA synthetic humanized construct R1ACR_591-1
atggtgtggtcccacgtggacgagcccgccctgggaccttacggctcttgggaggcagcaaaccctgattgcgtggccctgggcctgaccacaagcaagaataccaagatgatcctgctgctgttcgcctctatctgcgcctgtcaggtgctgtttatggccagccgggcccctagaatcacatgggaggccgtgtacctgccactgatcgagacaatcctgtatgccatggcctctacaggcaacggccagctgaggctgggcgacggacgcgtgctgccaatcgcacggtactgcagctggctgatcacctgtcctatcatgctgttccaggtggtgggcctgtatgatctgaagctgtggggcatctcctgcaagcacatggtcatggccgccgccctgatcaggaccgtgtttggcatcggcgccagcgtgacactggacgatcagatgagatgggtgcagttctttctgtcctacgccttctttttctttgagctgtactgtgcctatatgatcttcggcgccgccctgaagcagtttggcgaggtgaggaccacactgaactccatcgtgtattctagaatcctgctgctgagattcatcttctttctgtcttggaatagctttggcatcatctggctgctgagctccaccggcctgtgcatgatcagcgaggacgtgtccgtgctggcatacctggcagccgatatgctgtgcaagaatacctatggcatcatcaacgccaatacactgttccgcgtgctggacggcaagtggaccccagagacactggaggaggcagcatccggagtgaagaaggcaaaccccaatcacgtgggcgagacatatctggagaggatgatggcaggcatccgcggctctgccaagccagaggaggaccccgtggccgcc

SEQ ID NO: 44 - DNA, 819 bp, Artificial Sequence
  cDNA synthetic humanized construct R1ACR_053-1
atgggcctggacaccagctcccagacaaacatgatccagctgctgtttgcctccatctgcttctgtcaggtgctgtttatggcctctagggcaccaagagtgacctgggaggccgtgtacctgccactggtggagacagtgctgtatggcatggccgccagcggcaacggcaatctgaggatctccgacggccgcatggtgcctatggccaggttctgcagctggctgatcacctgtccaatcatgctgctgcaggtggtggtcatgcacgatgtggtgtggtttaacgtgtctaccaggtacatggtgctggccgccagcctgatccgcacagtgttcggcatcggcgccagcgtgaccacaaatgataccatgcggtgggtgcagtttctgctgtcctgcatctgttttgccttcgagatggtggtggcccaccagacatacaagggcgccctgaagaagttcgccgccgtgaagaccacactgaacggcgtggtgtatgcccggatcatgtttctgagattcctgttctttttctcttggaatgcctttcccatcatctgggccctgtctagcaccggcctgtgcctgatctccgaggacgcctccgtgctgtcttacctggtggccgatctgatctgtaagaatatctatggcatcaccaacgccaatacagtgttccgggtgctggacggagcatgggtgccagagacagaggagcacgcacagcctaaggatcacctgacatatctggagatgatgatgaaccacaatctgggcatcggcagacaccacgacgaggagctgcagatggaggtg

SEQ ID NO: 45 - DNA, 906 bp, Artificial Sequence
  cDNA synthetic humanized construct C1ACR_561-1
atggagatcaacaccacaagcccatacggatggtgggctgccgccaatccagagtgcgtggcaaaggcagcagtgaccgtggacatccagatctttcagctgtgctggtgtatcatctgcgcctgtcaggccttctttttcgccatccggtaccctaaggtgacctgggagttcgtgctggtgccactgacagaggccgtgatctacggcctgggctatagcggcatcggctatgtggtgctgcagtccggccggaccgtggcatggatgagaatctgccactggctgatcacagtgccagtgctgctgctgcagatcagcgacgtggcacacgtgcggtttcacggagtgaacctgagctccctgcaggtgcccatctccgtgctgatgatcgtgtttggcttctgtgccgaggtggccgatgaggagggcgtgaagtggatgtttttcgtgatcggcctggtgctgatgtttcaggtgctgttcgtgggctaccactctctgagcaggggcatcacccgcttctctgaggcaggaacagaggagggagcaatggtggcaaacaggctgagagccatgctggtgtttttcctgctgtcttggacactgcaccccctgtttttcgtgctgagcatcgagggcgcctgcgtgatgaccgagcctgtgacagtggtgctgtatatcgtgctggacctgtttgccaagaatatcttccagatcgtgctgtgggattccctgtgggtgaggctgaccggcaagtggaaggtggaggagctggtggtgcacgacgcatccgccgagctgggctacgacgaggattataagcacgatgagatcaacctgtctatcggcaatgtgcccgtgagcacagagtctcccgtgcctcgcggcgccggcgagggcgcc

SEQ ID NO: 46 - DNA, 888 bp, Artificial Sequence
  cDNA synthetic humanized construct R2ACR_853-1
atgagcgccgtggccgatccccagtttggcctgtacggcacctgggaggcctccaacatggattgcgccgccatgggccccaccattacctccaccaccaagatcttccagctgctgttcgcctgcatctgcgcctgtcaggtggtgttcatggccagcagggcccccagagtcacatgggaggccgtgtacctgcccctggtggagaccattctgtacagcgtggccgccaccggcaacggcaacatgagactcagcgacggcagggtggtccccatcgccagattctgcagctggctgatcacctgtcccatcatgctgttccaggtggtgggcatccatgagctgaagtggatgggcgtgagctgcaagaatatggtgatggccgccaccctgatccagaccgtgtacggcattggcgccagcgtgatcagcaacaacttcatgaagtggttcctgttcttcctgggctgcatctgcctggccttcgacttctttctggtcagcagcattttcgccgccggcctgaagaagtttggcgccttcaggtccaagctgaacaacatcgtgtacaacaggctgatgttcatgaggagcctgttcttcttcgcctgggtgagcttccccatcatctggacactggcccccaccggcatctgcatgatcaatgagaacgccaccgtgctggcctacctgatcgctgatatgctgtgcaagaacatgtacggcatcatcaacgccaataccctgttccaggtcctcaacggcagctggacccccgaaaccctggagtccagcaagctgcctcagaaaaacgaagtgcacgagacctacatcgatatggccctggatagctggaggggcaaggccagcgaggagacagaggct

SEQ ID NO: 47 - protein, 296 aa, Artificial Sequence
  synthetic rhodopsin (7 TM) domain (R1ACR_853)
MSAVADPQFGLYGTWEASNMDCAAMGPTITSTTKIFQLLFACICACQVVFMASRAPRVTWEAVYLPLVETILYSVAATGNGNMRLSDGRVVPIARFCSWLITCPIMLFQVVGIHELKWMGVSCKNMVMAATLIQTVYGIGASVISNNFMKWFLFFLGCICLAFDFFLVSSIFAAGLKKFGAFRSKLNNIVYNRLMFMRSLFFFAWVSFPIIWTLAPTGICMINENATVLAYLIADMLCKNMYGIINANTLFQVLNGSWTPETLESSKLPQKNEVHETYIDMALDSWRGKASEETEA

SEQ ID NO: 48 - protein, 885 aa, Artificial Sequence
  cDNA synthetic humanized construct R2ACR_142-1
ATGGCCACCTTTAACTCCACCTGGACCACCTACGGCGCCTGGGAAGCTGCTAACCCCGAGTGCGCCGAGAAGATGCACAGGTCCTCCGACGCTGTGCACGTGGTGCAGCTCACCTGGGCCATCGTGAGCTTCCTGCAGAGCATGTTCATGCTGTCCAGGTTCCCCAGGAACGCCTGGGAGGCCGTGTTCCTGCCCCTGGCCGAGACATGCCTCTATGGCCTGGCCTCCACCGGCAACGGCTACGTGACCATGGCCAACGGCAGAACACTGCCCTGGGGCAGGATGGCCAGCTGGCTGGCTACCTGCCCCATTATCATGAACCAGATCAACGCCGTGGCCACCGTGAACTTCCACGGCATCAGCCTGAACCGTATTCAAGTGTGCGCCAGCATCCTCACCATCGTGATGGGCGTGACCGCCTCCGTGACCGATGAACCCCCCATGCAGTGGGCTTTTTTCGGCCTGGGCACCCTGTGCCTGATCGTGGAGTACGTGATTACCACCATCATCATCTCCGCCGGCGTGGCCAAATTTCAGCCCTACGTGGAGGAGAGCGAGACCTGTAGGAAGGCCATCGGCAGGCTGTACCTGGTCAGGAACATGATCTTCAGCACCTGGGCCGCCTTCCCTATCGTGTGGCTGCTGTCCCCCCAAGGAGCCTGTGTGTTCGGCGAGGACGTGGTGCCCCTGCTGTACCTGATCGCCGACGCCCTGTGCAAGAACGTGTACGGCCTGATCTGGTATAACACCACCTGGGTGCTGCTGGGCGATGGAGTGTGGGACCCTACCAAGCACGTGGCCAAGCAAGCCCCTGCCCTGCAGCAGGAGATGACCGCCAACACCTCCGTGATTATGGCCCCTGCCGTGATCCAGGAGACCCCCTGT

SEQ ID NO: 49 - protein, 295 aa, Artificial Sequence
  synthetic rhodopsin (7 TM) domain (R2ACR_142-1)
MATFNSTWTTYGAWEAANPECAEKMHRSSDAVHVVQLTWAIVSFLQSMFMLSRFPRNAWEAVFLPLAETCLYGLASTGNGYVTMANGRTLPWGRMASWLATCPIIMNQINAVATVNFHGISLNRIQVCASILTIVMGVTASVTDEPPMQWAFFGLGTLCLIVEYVITTIIISAGVAKFQPYVEESETCRKAIGRLYLVRNMIFSTWAAFPIVWLLSPQGACVFGEDVVPLLYLIADALCKNVYGLIWYNTTWVLLGDGVWDPTKHVAKQAPALQQEMTANTSVIMAPAVIQETPC

SEQ ID NO: 50 - DNA, 915 bp, Artificial Sequence
  cDNA synthetic humanized construct (PsuACR_003-1)
atggaagccggagccaccatcgagcaaggcggcctggagtcctacgaccacaagatcacctacggcgtctgggaaatgcagcaccccgagtgctttgagaggggcatcgagagcggcgacgccgtgaagatcctgcagctgtgcttcgccgtgatctccatctgccaggtgatcttcatgttcaccagggcccccaaggtggcttgggagtccatctacctgcccctggctgagtgcatcacatacttcctggccttcaccggctacggagtgctgaggatgagcgacggcagaatcttcccctgggccaggatggccagctgggtgtgcacatgccctgtgatgctgggccaggtgagcgccatgggcctgatcaagtggaagagcgtgcccctgaacaatgtgatgatcgccgccagcctggtgagggtgtgcatgggcatcaccgccaccgtgacccccaatgacaacctgaagtggatgttcttcttcttcggcgtgacctgtctgctcttcgagtacacctgcgtcttcaccatcttccagctgaccatcaacgacttcgaggagatcggctcccccctgggcaatttcgtggccggaagggcccgtattttaaggatcatcttccacaccgcctggacctgcttccctgtgctgtgggtgctgagctccaccggcacctgcatgctggatgaaaacacctccgccacactgttcatgctggccgacgccgcctgcaagaacatgtacggcctcatcctgtggaacaccacctggggccatctgaacggcaagtgggacagggattacgtgaggagccagatcgaggccaaggaagagaagcctgccgacaaggagatgtcccaggtgcctcccgccgctcaggatgatggcgtgaaggtcttcggcagg

SEQ ID NO: 51 - DNA, 888 bp, Artificial Sequence
  cDNA synthetic humanized construct R2ACR_853-2
atgagcgccgtggccgatccccagttcggcctgtacggcacctgggaggccagcaacatggattgcgccgccatgggccccaccatcaccagcaccaccaagatcttccagctgctgttcgcctgcatctgcgcctgccaggtggtgttcatggccagcagagcccccagagtgacctgggaggccgtgtacctgcccctggtggagaccatcctgtacagcgtggccgccaccggcaacggcaacatgagactgagcgacggcagagtggtgcccatcgccagattctgcagctggctgatcacctgccccatcatgctgttccaggtggtgggcatccacgagctgaagtggatgggcgtgagctgcaagaacatggtgatggccgccaccctgatccagaccgtgtacggcatcggcgccagcgtgatcagcaacaacttcatgaagtggttcctgttcttcctgggctgcatctgcctggccttcgacttcttcctggtgagcagcatcttcgccgccggcctgaagaagttcggcgccttcagaagcaagctgaacaacatcgtgtacaacagactgatgttcatgagaagcctgttcttcttcgcctgggtgagcttccccatcatctggaccctggcccccaccggcatctgcatgatcaacgagaacgccaccgtgctggcctacctgatcgccgatatgctgtgcaagaacatgtacggcatcatcaacgccaacaccctgttccaggtgctgaacggcagctggacccccgagaccctggagagcagcaagctgccccagaagaacgaggtgcacgagacctacatcgatatggccctggatagctggagaggcaaggccagcgaggagaccgaggcc

SEQ ID NO: 52 - DNA, 885 bp, Artificial Sequence
  cDNA synthetic humanized construct R2ACR_142-2
atggccacctttaactccacctggaccacctacggcgcctgggaagctgctaaccccgagtgcgccgagaagatgcacaggtcctccgacgctgtgcacgtggtgcagctcacctgggccatcgtgagcttcctgcagagcatgttcatgctgtccaggttccccaggaacgcctgggaggccgtgttcctgcccctggccgagacatgcctctatggcctggcctccaccggcaacggctacgtgaccatggccaacggcagaacactgccctggggcaggatggccagctggctggctacctgccccattatcatgaaccagatcaacgccgtggccaccgtgaacttccacggcatcagcctgaaccgtattcaagtgtgcgccagcatcctcaccatcgtgatgggcgtgaccgcctccgtgaccgatgaaccccccatgcagtgggcttttttcggcctgggcaccctgtgcctgatcgtggagtacgtgattaccaccatcatcatctccgccggcgtggccaaatttcagccctacgtggaggagagcgagacctgtaggaaggccatcggcaggctgtacctggtcaggaacatgatcttcagcacctgggccgccttccctatcgtgtggctgctgtccccccaaggagcctgtgtgttcggcgaggacgtggtgcccctgctgtacctgatcgccgacgccctgtgcaagaacgtgtacggcctgatctggtataacaccacctgggtgctgctgggcgatggagtgtgggaccctaccaagcacgtggccaagcaagcccctgccctgcagcaggagatgaccgccaacacctccgtgattatggcccctgccgtgatccaggagaccccctgt

SEQ ID NO: 53 - DNA, 861 bp, Artificial Sequence
  cDNA synthetic humanized construct R2ACR_041-1
atgggcctgaccgtgacatctaacacaaaggtgttccagctgctgtttgcctgcgtgtgcgcctgccaggtgctgttcatggcaagcagggcaccaagagtgacatgggaggccgtgtacctgcccctggtggagacaatcctgtatgccatggccgccagcggcaatggctccatgaggctggcagacggacgcgtgatccctatcggccggttctgttcctggctgatcacatgcccaatcatgctgtttcaggtggtgggcatccacgagctgaagtggtctggcgtgagctgtaagaacatggtcatggccgccagcctgatcagaaccgtgtacggaatcggagcatccgtggtggccaatgacatcatgaagtgggtgctgtatgtgctgggctgcgtgtgcctggccttcgagtttctggtggtgcactccatcttttctgccggcctgaagaagttcgccgcctttaggtctaccctgaacgatggcgtgtacaatcggctgctgttcatgagagccctgttctttaccgcctggacatcttttccagtgatctgggtgctggccagcacaggcatctgtctgatcgacgagaacgtgaccgtgctggcctacctgatcgccgatatgctgtgcaagaatatgtatggcatcatcaacgccaatacactgttccgggtgctggacggctcctggaccccagagacactggagacaagcaagaacccccctaagcacgaggtgcacgagacatatatcgatatggccatgcaccacatgaggcccagcaagtccaagacaagggaggcagcaatcgagctggaggagcacaccgccgatccttttgccgagggc

SEQ ID NO: 54 - DNA, 888 bp, Artificial Sequence
  cDNA synthetic humanized R2ACR1
atgagcgccgtggccgacccccagttcggcctgtacggcacctgggaggccagcaacatggactgcgccgccatgggccccaccatcaccagcaccaccaagatcttccagctgctgttcgcctgcatctgcgcctgccaggtggtgttcatggccagccgcgccccccgcgtgacctgggaggccgtgtacctgcccctggtggagaccatcctgtacagcgtggccgccaccggcaacggcaacatgcgcctgagcgacggccgcgtggtgcccatcgcccgcttctgcagctggctgatcacctgccccatcatgctgttccaggtggtgggcatccacgagctgaagtggatgggcgtgagctgcaagaacatggtgatggccgccaccctgatccagaccgtgtacggcatcggcgccagcgtgatcagcaacaacttcatgaagtggttcctgttcttcctgggctgcatctgcctggccttcgacttcttcctggtgagcagcatcttcgccgccggcctgaagaagttcggcgccttccgcagcaagctgaacaacatcgtgtacaaccgcctgatgttcatgcgcagcctgttcttcttcgcctgggtgagcttccccatcatctggaccctggcccccaccggcatctgcatgatcaacgagaacgccaccgtgctggcctacctgatcgccgacatgctgtgcaagaacatgtacggcatcatcaacgccaacaccctgttccaggtgctgaacggcagctggacccccgagaccctggagagcagcaagctgccccagaagaacgaggtgcacgagacctacatcgacatggccctggacagctggcgcggcaaggccagcgaggagaccgaggcc

SEQ ID NO: 55 - DNA, 885 bp, Artificial Sequence
  cDNA synthetic humanized R2ACR2
atggccaccttcaacagcacctggaccacctacggcgcctgggaggccgccaaccccgagtgcgccgagaagatgcaccgcagcagcgacgccgtgcacgtggtgcagctgacctgggccatcgtgagcttcctgcagagcatgttcatgctgagccgcttcccccgcaacgcctgggaggccgtgttcctgcccctggccgagacctgcctgtacggcctggccagcaccggcaacggctacgtgaccatggccaacggccgcaccctgccctggggccgcatggccagctggctggccacctgccccatcatcatgaaccagatcaacgccgtggccaccgtgaacttccacggcatcagcctgaaccgcatccaggtgtgcgccagcatcctgaccatcgtgatgggcgtgaccgccagcgtgaccgacgagccccccatgcagtgggccttcttcggcctgggcaccctgtgcctgatcgtggagtacgtgatcaccaccatcatcatcagcgccggcgtggccaagttccagccctacgtggaggagagcgagacctgccgcaaggccatcggccgcctgtacctggtgcgcaacatgatcttcagcacctgggccgccttccccatcgtgtggctgctgagcccccagggcgcctgcgtgttcggcgaggacgtggtgcccctgctgtacctgatcgccgacgccctgtgcaagaacgtgtacggcctgatctggtacaacaccacctgggtgctgctgggcgacggcgtgtgggaccccaccaagcacgtggccaagcaggcccccgccctgcagcaggagatgaccgccaacaccagcgtgatcatggcccccgccgtgatccaggagaccccctgc

SEQ ID NO: 56 - protein, 288 aa, Artificial Sequence
  synthetic humanized rhodopsin (7 TM) domain (GcACR_201)
MEAADEEDVGGGDLWNVNTGCLYGNLASASPLVRHFELAWAVCCLTQAFFKFTQWPRAAWEGVYIPLCETVQYSLLFFKLGYIQVGVNQGAYLPWVRYSFWMITTPVLLNQIQQICEVKIWGARMGPVQTWANIAMVTLGISGALIQNPTMKWLCFVAGTLLGALIFLVAFLIFHKASEQFKEIGHPKADRVVKYLWYLQVCFFSSWSVFPLLWIISAQGTCIANEDIVSSIHIPADILAKNVFGLIIWHIRFHLCDGYWDIERFKQKLLQDKLLALFDNPEELRQDH

SEQ ID NO: 57 - protein, 285 aa, Artificial Sequence
  synthetic humanized rhodopsin (7 TM) domain (HpACR_213)
MTVIYGQWAAQNPGCVSMTNIDLGVRTFELCWGIMCACQAVFFALRYPRATWEFVMVPLTECFIYGLGYSEIGYILLWDGRQVLWTRTVLWLATVPILLNQINAMAAVHIYGVDLNIMQMYFSTLMICLGHTAALTHNQSLKWLFFIMAMAIFGVICFINYYTFAAAEKHYKESGSENDLKIAKRIRLLAIIFFTSWTMYPLFFVLSVEGACVASESVILVCFALADLLSKNVFGVLFWDTLWNMQDGKWTSYGTVTFFKTTDSVTKTAISTAMEEEYSKQKVSR

SEQ ID NO: 58 - protein, 291 aa, Artificial Sequence
  synthetic humanized rhodopsin (7 TM) domain (HaACR_359)
MATPAPAGAPLTTTPVPYGQWEANNPACVALFTMPAQVQTLMLCWCIICSCQAIFLMSRWPRVGWELVAAPAIESIVYGLGYQGIGYVKMASGQPLAYARFFMMITITPMILLDINKLASVRLLGLNMNSMQLGANVLLWMFWQASTMSTLVGLKWVFFVLGLLCLGLVLGTSFMIFAAARQSFLLKGTGAGDWVAQRITYLQMAFLPTWTAYMVLFVLSGEGACIAGEGVILPIAIVMDLFQKNVHGLIIWSTFWNFLGGSWNPERLAEERAPSRIAGLKVIENNAHPVD

SEQ ID NO: 59 - protein, 300 aa, Artificial Sequence
  synthetic humanized rhodopsin (7 TM) domain (HrACR_495)
MATPGPAAVPSMTTTPAPYGTWEANNPSCVALFNSSPQIQTLMLCWCIICTCQSIFLMSRWPRVGWELVAAPAVECIVYGLGYQGIGYVKMASGQQLAYARFFMMITVTLMILIDVNKLASVRLLGLNMNSMQLAANLLLWMFWQASTMSTLSGLKWVLFVLGLICLGIVLGTSFMIFSAARQGFLDKNTGAGDWVAQRIVFLEVVFFPTWAGYMVLYVISGEGACIAPETVIIPVAIVLDLFQKNVHGLIIWSTFWNFLGGSWNPGRLAEERAPSRLAGLKVIENTAHGFDDEALSEGF

SEQ ID NO: 60 - protein, 300 aa, Artificial Sequence
  synthetic humanized rhodopsin (7 TM) domain (PsuACR_353)
MEVEMEMEMEGLGNSTSMFEEVVGCDLGASPVGETEMHVVQWIGSFICIFQCLWFLSKWPNFTWDTPYIPAVEAVGYGLMANGFGYAELFEGERHVPWLRYSIWVLSTPVLLWDIQDILDAFYRGTRLAPVQSLANLFLLCFGFIGAIEHVLAVKYLCYSIGVMMLGVVFFCAYNIVGQAVANFEATDTAQGRLVAFRLKLIACIFMTSFSGYPILWILSNQGLCLVPEMIIEVVHIPLDLIAKNLFTLLTWHTHYILLNGDWSCEAMVARLQEQNDIEMAQQPREAGQLRKDLMQKAQG

SEQ ID NO: 61 - protein, 306 aa, Artificial Sequence
  synthetic humanized rhodopsin (7 TM) domain (RaACR_687)
MGDMSHIETVYYVNGTEVDPWTHRCQLGVPGGSQGIHTFQLTWSLICTAQAIFLMTKWPKVGWEAIYMPCCEALSYGLLSAGHGYLKLHGEWHYIPWIRYAAWMVTTPVLLNQVQGVLDVKVGHLRLAPIQSLANLIMIVMGVSAHLQENIAWKWVFFLIGCVMALVIYWVAWLVFSTQIKYYAETKTAVGDRVVFQLKLIAVAFYVGWSAFPLIWMLSSEGLCAIEEHIAAAAHIPADVLSKNIFGLLMWHTRWIVLGGYWDVNKFKETIAQQKLEEAKGEETPQEKRKALMGQAEACFKMADAC

SEQ ID NO: 62 - protein, 312 aa, Artificial Sequence
  synthetic humanized rhodopsin (7 TM) domain (RlACR_497)
MEYQSLDDFPLETLYFRNGTEVDPYDHRCKLGIPGGSQGIHTFQLVWALLCAAQALFLFSKWPKVGWEAMYMPCCEALSYGLLSSGYGYMKLHGEFHYLPWIRYAAWIVTTPVLLNQVQGMIDIKIGSLRLAPIQSLANLIMIIMGVSAHLQENHAWKWLFFFVGCVMCAVIYYIAWLQFAKVNELYAATKTEIGDKVCFQLKLIMFCFYVGWSMFSVIWLLSSEGLCLIEEHISAAAHIPGDILSKNSFGLLMWYTRWITLGGAWDINKFLEKQAKEKAEAVLTPAEKRKAIMKQAEACFSMADACLQQAN

SEQ ID NO: 63 - protein, 307 aa, Artificial Sequence
  synthetic humanized rhodopsin (7 TM) domain (RsACR_665)
MDYTSLDDFPLETLYFRNGTEVDPYDHRCKLGIPGGSQGIHTFQLVWALLCAAQALFLFSKWPKVGWEAMYMPCCEALSYGLLSSGYGYMKLHGEFHYLPWIRYAAWLVTTPVLLNQVQGMIDIKIGSLRLAPIQSLANLIMIIMGVSAHLQENHAWKWLFFFVGCVMCAVIYYIAWLQFAKVNELYAATKTEIGDKVCFQLKLIMFCFYVGWSMFSVIWLLSSEGLCLIEEHISAAAHIPGDILSKNSFGLLMWYTRWITLGGAWDINKFLEKQAKEKAEAVLTPAEKRKAIMKQAEACFSMADAC

SEQ ID NO: 64 - protein, 285 aa, Artificial Sequence
  synthetic humanized rhodopsin (7 TM) domain (R1ACR_653)
MPYDNYGWWERENADCISKMDAKLDQMHLVQLAMSMLCFCQLVFMTSRFPKQAWEAIYLPAAECVLYAMAASGNGYLRLANGRLLPWARMASWLATCPVMLNQINTISPLKLYGINLNNVQIALSVVRVVNGVSASSVDDDSLKWGFFFMGVFCLVIEFSIAYNIFALGMAKFKEAGKDSDICARACDRLQVLRGIFFSAWAMFPIIWLFSPQGTCLLGEDVIPLLYLFADLVCKNCFGILWYDTTWRLLDGKGWNPELQLTDNEVEELVHAKEEVEDHHHPHDA

SEQ ID NO: 65 - protein, 297 aa, Artificial Sequence
  synthetic humanized rhodopsin (7 TM) domain (R1ACR_6367)
MSAITDPALGLYGTWEAANKECVDKGVEFSSNTRMIVLLFASVCFCQTVYMTSRAPRVNWEGCYLPLCETFLYGLAASGNGMIRLSNGRMVPMARFCSWLVTCPIMLMSVLAIHDVRWMGVPVKNMVIAATLIRTVLGIGASSTITETLRWVQYCIACAVFVFELTMVNQLFIAGLQKFGVVKTKLNDMVYMRIMILRALFFFSWTAFPVIWILSSTGFCVIGEDGSVLAYLVADFLCKNVYGIINTNTLFQVLDGEWKPETRDTDGKIATNRHAHIGETYLEMLLARIRRHREGEL

SEQ ID NO: 66 - protein, 305 aa, Artificial Sequence
  synthetic rhodopsin R1ACR_591
MVWSHVDEPALGPYGSWEAANPDCVALGLTTSKNTKMILLLFASICACQVLFMASRAPRITWEAVYLPLIETILYAMASTGNGQLRLGDGRVLPIARYCSWLITCPIMLFQVVGLYDLKLWGISCKHMVMAAALIRTVFGIGASVTLDDQMRWVQFFLSYAFFFFELYCAYMIFGAALKQFGEVRTTLNSIVYSRILLLRFIFFLSWNSFGIIWLLSSTGLCMISEDVSVLAYLAADMLCKNTYGIINANTLFRVLDGKWTPETLEEAASGVKKANPNHVGETYLERMMAGIRGSAKPEEDPVAA

SEQ ID NO: 67 - protein, 240 aa, Artificial Sequence
  synthetic humanized rhodopsin (7 TM) domain (R1ACR_053)
MGLDTSSQTNMIQLLFASICFCQVLFMASRAPRVTWEAVYLPLVETVLYGMAASGNGNLRISDGRMVPMARFCSWLITCPIMLLQVVVMHDVVWFNVSTRYMVLAASLIRTVFGIGASVTTNDTMRWVQFLLSCICFAFEMVVAHQTYKGALKKFAAVKTTLNGVVYARIMFLRFLFFFSWNAFPIIWALSSTGLCLISEDASVLSYLVADLICKNIYGITNANTVFRVLDGAWVPETEE

SEQ ID NO: 68 - protein, 302 aa, Artificial Sequence
  synthetic humanized rhodopsin (7 TM) domain (C1ACR_561)
MEINTTSPYGWWAAANPECVAKAAVTVDIQIFQLCWCIICACQAFFFAIRYPKVTWEFVLVPLTEAVIYGLGYSGIGYVVLQSGRTVAWMRICHWLITVPVLLLQISDVAHVRFHGVNLSSLQVPISVLMIVFGFCAEVADEEGVKWMFFVIGLVLMFQVLFVGYHSLSRGITRFSEAGTEEGAMVANRLRAMLVFFLLSWTLHPLFFVLSIEGACVMTEPVTVVLYIVLDLFAKNIFQIVLWDSLWVRLTGKWKVEELVVHDASAELGYDEDYKHDEINLSIGNVPVSTESPVPRGAGEGA

SEQ ID NO: 69 - protein, 287 aa, Artificial Sequence
  synthetic humanized rhodopsin (7 TM) domain (R2ACR_041)
MGLTVTSNTKVFQLLFACVCACQVLFMASRAPRVTWEAVYLPLVETILYAMAASGNGSMRLADGRVIPIGRFCSWLITCPIMLFQVVGIHELKWSGVSCKNMVMAASLIRTVYGIGASVVANDIMKWVLYVLGCVCLAFEFLVVHSIFSAGLKKFAAFRSTLNDGVYNRLLFMRALFFTAWTSFPVIWVLASTGICLIDENVTVLAYLIADMLCKNMYGIINANTLFRVLDGSWTPETLETSKNPPKHEVHETYIDMAMHHMRPSKSKTREAAIELEEHTADPFAEG

SEQ ID NO: 70 - protein, 864 aa, Artificial Sequence
  synthetic humanized rhodopsin (7 TM) domain (GcACR_201)
ATGGAGGCCGCCGACGAGGAGGACGTGGGCGGCGGCGACCTGTGGAACGTGAACACCGGCTGCCTGTACGGCAACCTGGCCAGCGCCAGCCCCCTGGTGCGCCACTTCGAGCTGGCCTGGGCCGTGTGCTGCCTGACCCAGGCCTTCTTCAAGTTCACCCAGTGGCCCCGCGCCGCCTGGGAGGGCGTGTACATCCCCCTGTGCGAGACCGTGCAGTACAGCCTGCTGTTCTTCAAGCTGGGCTACATCCAGGTGGGCGTGAACCAGGGCGCCTACCTGCCCTGGGTGCGCTACAGCTTCTGGATGATCACCACCCCCGTGCTGCTGAACCAGATCCAGCAGATCTGCGAGGTGAAGATCTGGGGCGCCCGCATGGGCCCCGTGCAGACCTGGGCCAACATCGCCATGGTGACCCTGGGCATCAGCGGCGCCCTGATCCAGAACCCCACCATGAAGTGGCTGTGCTTCGTGGCCGGCACCCTGCTGGGCGCCCTGATCTTCCTGGTGGCCTTCCTGATCTTCCACAAGGCCAGCGAGCAGTTCAAGGAGATCGGCCACCCCAAGGCCGACCGCGTGGTGAAGTACCTGTGGTACCTGCAGGTGTGCTTCTTCAGCAGCTGGAGCGTGTTCCCCCTGCTGTGGATCATCAGCGCCCAGGGCACCTGCATCGCCAACGAGGACATCGTGAGCAGCATCCACATCCCCGCCGACATCCTGGCCAAGAACGTGTTCGGCCTGATCATCTGGCACATCCGCTTCCACCTGTGCGACGGCTACTGGGACATCGAGCGCTTCAAGCAGAAGCTGCTGCAGGACAAGCTGCTGGCCCTGTTCGACAACCCCGAGGAGCTGCGCCAGGACCAC

SEQ ID NO: 71 - DNA, 864 bp, Artificial Sequence
  synthetic humanized rhodopsin (7 TM) domain (GcACR_201)
atggaggccgccgacgaggaggacgtgggcggcggcgacctgtggaacgtgaacaccggctgcctgtacggcaacctggccagcgccagccccctggtgagacacttcgagctggcctgggccgtgtgctgcctgacccaggccttcttcaagttcacccagtggcccagagccgcctgggagggcgtgtacatccccctgtgcgagaccgtgcagtacagcctgctgttcttcaagctgggctacatccaggtgggcgtgaaccagggcgcctacctgccctgggtgagatacagcttctggatgatcaccacccccgtgctgctgaaccagatccagcagatctgcgaggtgaagatctggggcgccagaatgggccccgtgcagacctgggccaacatcgccatggtgaccctgggcatcagcggcgccctgatccagaaccccaccatgaagtggctgtgcttcgtggccggcaccctgctgggcgccctgatcttcctggtggccttcctgatcttccacaaggccagcgagcagttcaaggagatcggccaccccaaggccgacagagtggtgaagtacctgtggtacctgcaggtgtgcttcttcagcagctggagcgtgttccccctgctgtggatcatcagcgcccagggcacctgcatcgccaacgaggacatcgtgagcagcatccacatccccgccgacatcctggccaagaacgtgttcggcctgatcatctggcacatcagattccacctgtgcgacggctactgggacatcgagagattcaagcagaagctgctgcaggacaagctgctggccctgttcgacaaccccgaggagctgagacaggaccac

SEQ ID NO: 72 - DNA, 1710 bp, Artificial Sequence
  synthetic humanized rhodopsin (7 TM) domain (HpACR_213)
atgaccgtgatctacggccagtgggccgcccagaaccccggctgcgtgagcatgaccaacatcgacctgggcgtgcgcaccttcgagctgtgctggggcatcatgtgcgcctgccaggccgtgttcttcgccctgcgctacccccgcgccacctgggagttcgtgatggtgcccctgaccgagtgcttcatctacggcctgggctacagcgagatcggctacatcctgctgtgggacggccgccaggtgctgtggacccgcaccgtgctgtggctggccaccgtgcccatcctgctgaaccagatcaacgccatggccgccgtgcacatctacggcgtggacctgaacatcatgcagatgtacttcagcaccctgatgatctgcctgggccacaccgccgccctgacccacaaccagagcctgaagtggctgttcttcatcatggccatggccatcttcggcgtgatctgcttcatcaactactacaccttcgccgccgccgagaagcactacaaggagagcggcagcgagaacgacctgaagatcgccaagcgcatccgcctgctggccatcatcttcttcaccagctggaccatgtaccccctgttcttcgtgctgagcgtggagggcgcctgcgtggccagcgagagcgtgatcctggtgtgcttcgccctggccgacctgctgagcaagaacgtgttcggcgtgctgttctgggacaccctgtggaacatgcaggacggcaagtggaccagctacggcaccgtgaccttcttcaagaccaccgacagcgtgaccaagaccgccatcagcaccgccatggaggaggagtacagcaagcagaaggtgagccgcatgaccgtgatctacggccagtgggccgcccagaaccccggctgcgtgagcatgaccaacatcgacctgggcgtgcgcaccttcgagctgtgctggggcatcatgtgcgcctgccaggccgtgttcttcgccctgcgctacccccgcgccacctgggagttcgtgatggtgcccctgaccgagtgcttcatctacggcctgggctacagcgagatcggctacatcctgctgtgggacggccgccaggtgctgtggacccgcaccgtgctgtggctggccaccgtgcccatcctgctgaaccagatcaacgccatggccgccgtgcacatctacggcgtggacctgaacatcatgcagatgtacttcagcaccctgatgatctgcctgggccacaccgccgccctgacccacaaccagagcctgaagtggctgttcttcatcatggccatggccatcttcggcgtgatctgcttcatcaactactacaccttcgccgccgccgagaagcactacaaggagagcggcagcgagaacgacctgaagatcgccaagcgcatccgcctgctggccatcatcttcttcaccagctggaccatgtaccccctgttcttcgtgctgagcgtggagggcgcctgcgtggccagcgagagcgtgatcctggtgtgcttcgccctggccgacctgctgagcaagaacgtgttcggcgtgctgttctgggacaccctgtggaacatgcaggacggcaagtggaccagctacggcaccgtgaccttcttcaagaccaccgacagcgtgaccaagaccgccatcagcaccgccatggaggaggagtacagcaagcagaaggtgagccgc

SEQ ID NO: 73 - DNA, 855 bp, Artificial Sequence
  synthetic humanized rhodopsin (7 TM) domain (HpACR_213)
atgaccgtgatctacggccagtgggccgcccagaaccccggctgcgtgagcatgaccaacatcgacctgggcgtgagaaccttcgagctgtgctggggcatcatgtgcgcctgccaggccgtgttcttcgccctgagataccccagagccacctgggagttcgtgatggtgcccctgaccgagtgcttcatctacggcctgggctacagcgagatcggctacatcctgctgtgggacggcagacaggtgctgtggaccagaaccgtgctgtggctggccaccgtgcccatcctgctgaaccagatcaacgccatggccgccgtgcacatctacggcgtggacctgaacatcatgcagatgtacttcagcaccctgatgatctgcctgggccacaccgccgccctgacccacaaccagagcctgaagtggctgttcttcatcatggccatggccatcttcggcgtgatctgcttcatcaactactacaccttcgccgccgccgagaagcactacaaggagagcggcagcgagaacgacctgaagatcgccaagagaatcagactgctggccatcatcttcttcaccagctggaccatgtaccccctgttcttcgtgctgagcgtggagggcgcctgcgtggccagcgagagcgtgatcctggtgtgcttcgccctggccgacctgctgagcaagaacgtgttcggcgtgctgttctgggacaccctgtggaacatgcaggacggcaagtggaccagctacggcaccgtgaccttcttcaagaccaccgacagcgtgaccaagaccgccatcagcaccgccatggaggaggagtacagcaagcagaaggtgagcaga

SEQ ID NO: 74 - DNA, 873 bp, Artificial Sequence
  synthetic humanized rhodopsin (7 TM) domain (HaACR_359)
atggccacccccgcccccgccggcgcccccctgaccaccacccccgtgccctacggccagtgggaggccaacaaccccgcctgcgtggccctgttcaccatgcccgcccaggtgcagaccctgatgctgtgctggtgcatcatctgcagctgccaggccatcttcctgatgagccgctggccccgcgtgggctgggagctggtggccgcccccgccatcgagagcatcgtgtacggcctgggctaccagggcatcggctacgtgaagatggccagcggccagcccctggcctacgcccgcttcttcatgatgatcaccatcacccccatgatcctgctggacatcaacaagctggccagcgtgcgcctgctgggcctgaacatgaacagcatgcagctgggcgccaacgtgctgctgtggatgttctggcaggccagcaccatgagcaccctggtgggcctgaagtgggtgttcttcgtgctgggcctgctgtgcctgggcctggtgctgggcaccagcttcatgatcttcgccgccgcccgccagagcttcctgctgaagggcaccggcgccggcgactgggtggcccagcgcatcacctacctgcagatggccttcctgcccacctggaccgcctacatggtgctgttcgtgctgagcggcgagggcgcctgcatcgccggcgagggcgtgatcctgcccatcgccatcgtgatggacctgttccagaagaacgtgcacggcctgatcatctggagcaccttctggaacttcctgggcggcagctggaaccccgagcgcctggccgaggagcgcgcccccagccgcatcgccggcctgaaggtgatcgagaacaacgcccaccccgtggac

SEQ ID NO: 75 - DNA, 873 bp, Artificial Sequence
  synthetic humanized rhodopsin (7 TM) domain (HaACR_359)
atggccacccccgcccccgccggcgcccccctgaccaccacccccgtgccctacggccagtgggaggccaacaaccccgcctgcgtggccctgttcaccatgcccgcccaggtgcagaccctgatgctgtgctggtgcatcatctgcagctgccaggccatcttcctgatgagcagatggcccagagtgggctgggagctggtggccgcccccgccatcgagagcatcgtgtacggcctgggctaccagggcatcggctacgtgaagatggccagcggccagcccctggcctacgccagattcttcatgatgatcaccatcacccccatgatcctgctggacatcaacaagctggccagcgtgagactgctgggcctgaacatgaacagcatgcagctgggcgccaacgtgctgctgtggatgttctggcaggccagcaccatgagcaccctggtgggcctgaagtgggtgttcttcgtgctgggcctgctgtgcctgggcctggtgctgggcaccagcttcatgatcttcgccgccgccagacagagcttcctgctgaagggcaccggcgccggcgactgggtggcccagagaatcacctacctgcagatggccttcctgcccacctggaccgcctacatggtgctgttcgtgctgagcggcgagggcgcctgcatcgccggcgagggcgtgatcctgcccatcgccatcgtgatggacctgttccagaagaacgtgcacggcctgatcatctggagcaccttctggaacttcctgggcggcagctggaaccccgagagactggccgaggagagagcccccagcagaatcgccggcctgaaggtgatcgagaacaacgcccaccccgtggac

SEQ ID NO: 76 - DNA, 900 bp, Artificial Sequence
  synthetic humanized rhodopsin (7 TM) domain (HrACR_495)
atggccacccccggccccgccgccgtgcccagcatgaccaccacccccgccccctacggcacctgggaggccaacaaccccagctgcgtggccctgttcaacagcagcccccagatccagaccctgatgctgtgctggtgcatcatctgcacctgccagagcatcttcctgatgagccgctggccccgcgtgggctgggagctggtggccgcccccgccgtggagtgcatcgtgtacggcctgggctaccagggcatcggctacgtgaagatggccagcggccagcagctggcctacgcccgcttcttcatgatgatcaccgtgaccctgatgatcctgatcgacgtgaacaagctggccagcgtgcgcctgctgggcctgaacatgaacagcatgcagctggccgccaacctgctgctgtggatgttctggcaggccagcaccatgagcaccctgagcggcctgaagtgggtgctgttcgtgctgggcctgatctgcctgggcatcgtgctgggcaccagcttcatgatcttcagcgccgcccgccagggcttcctggacaagaacaccggcgccggcgactgggtggcccagcgcatcgtgttcctggaggtggtgttcttccccacctgggccggctacatggtgctgtacgtgatcagcggcgagggcgcctgcatcgcccccgagaccgtgatcatccccgtggccatcgtgctggacctgttccagaagaacgtgcacggcctgatcatctggagcaccttctggaacttcctgggcggcagctggaaccccggccgcctggccgaggagcgcgcccccagccgcctggccggcctgaaggtgatcgagaacaccgcccacggcttcgacgacgaggccctgagcgagggcttc

SEQ ID NO: 77 - DNA, 900 bp, Artificial Sequence
  synthetic humanized rhodopsin (7 TM) domain (HrACR_495)
atggccacccccggccccgccgccgtgcccagcatgaccaccacccccgccccctacggcacctgggaggccaacaaccccagctgcgtggccctgttcaacagcagcccccagatccagaccctgatgctgtgctggtgcatcatctgcacctgccagagcatcttcctgatgagcagatggcccagagtgggctgggagctggtggccgcccccgccgtggagtgcatcgtgtacggcctgggctaccagggcatcggctacgtgaagatggccagcggccagcagctggcctacgccagattcttcatgatgatcaccgtgaccctgatgatcctgatcgacgtgaacaagctggccagcgtgagactgctgggcctgaacatgaacagcatgcagctggccgccaacctgctgctgtggatgttctggcaggccagcaccatgagcaccctgagcggcctgaagtgggtgctgttcgtgctgggcctgatctgcctgggcatcgtgctgggcaccagcttcatgatcttcagcgccgccagacagggcttcctggacaagaacaccggcgccggcgactgggtggcccagagaatcgtgttcctggaggtggtgttcttccccacctgggccggctacatggtgctgtacgtgatcagcggcgagggcgcctgcatcgcccccgagaccgtgatcatccccgtggccatcgtgctggacctgttccagaagaacgtgcacggcctgatcatctggagcaccttctggaacttcctgggcggcagctggaaccccggcagactggccgaggagagagcccccagcagactggccggcctgaaggtgatcgagaacaccgcccacggcttcgacgacgaggccctgagcgagggcttc

SEQ ID NO: 78 - DNA, 900 bp, Artificial Sequence
  synthetic humanized rhodopsin (7 TM) domain (PsuACR_353)
atggaggtggagatggagatggagatggagggcctgggcaacagcaccagcatgttcgaggaggtggtgggctgcgacctgggcgccagccccgtgggcgagaccgagatgcacgtggtgcagtggatcggcagcttcatctgcatcttccagtgcctgtggttcctgagcaagtggcccaacttcacctgggacaccccctacatccccgccgtggaggccgtgggctacggcctgatggccaacggcttcggctacgccgagctgttcgagggcgagcgccacgtgccctggctgcgctacagcatctgggtgctgagcacccccgtgctgctgtgggacatccaggacatcctggacgccttctaccgcggcacccgcctggcccccgtgcagagcctggccaacctgttcctgctgtgcttcggcttcatcggcgccatcgagcacgtgctggccgtgaagtacctgtgctacagcatcggcgtgatgatgctgggcgtggtgttcttctgcgcctacaacatcgtgggccaggccgtggccaacttcgaggccaccgacaccgcccagggccgcctggtggccttccgcctgaagctgatcgcctgcatcttcatgaccagcttcagcggctaccccatcctgtggatcctgagcaaccagggcctgtgcctggtgcccgagatgatcatcgaggtggtgcacatccccctggacctgatcgccaagaacctgttcaccctgctgacctggcacacccactacatcctgctgaacggcgactggagctgcgaggccatggtggcccgcctgcaggagcagaacgacatcgagatggcccagcagccccgcgaggccggccagctgcgcaaggacctgatgcagaaggcccagggc

SEQ ID NO: 79 - DNA, 900 bp, Artificial Sequence
  synthetic humanized rhodopsin (7 TM) domain (PsuACR_353)
atggaggtggagatggagatggagatggagggcctgggcaacagcaccagcatgttcgaggaggtggtgggctgcgacctgggcgccagccccgtgggcgagaccgagatgcacgtggtgcagtggatcggcagcttcatctgcatcttccagtgcctgtggttcctgagcaagtggcccaacttcacctgggacaccccctacatccccgccgtggaggccgtgggctacggcctgatggccaacggcttcggctacgccgagctgttcgagggcgagagacacgtgccctggctgagatacagcatctgggtgctgagcacccccgtgctgctgtgggacatccaggacatcctggacgccttctacagaggcaccagactggcccccgtgcagagcctggccaacctgttcctgctgtgcttcggcttcatcggcgccatcgagcacgtgctggccgtgaagtacctgtgctacagcatcggcgtgatgatgctgggcgtggtgttcttctgcgcctacaacatcgtgggccaggccgtggccaacttcgaggccaccgacaccgcccagggcagactggtggccttcagactgaagctgatcgcctgcatcttcatgaccagcttcagcggctaccccatcctgtggatcctgagcaaccagggcctgtgcctggtgcccgagatgatcatcgaggtggtgcacatccccctggacctgatcgccaagaacctgttcaccctgctgacctggcacacccactacatcctgctgaacggcgactggagctgcgaggccatggtggccagactgcaggagcagaacgacatcgagatggcccagcagcccagagaggccggccagctgagaaaggacctgatgcagaaggcccagggc

SEQ ID NO: 80 - DNA, 918 bp, Artificial Sequence
  synthetic humanized rhodopsin (7 TM) domain (RaACR_687)
atgggcgacatgagccacatcgagaccgtgtactacgtgaacggcaccgaggtggacccctggacccaccgctgccagctgggcgtgcccggcggcagccagggcatccacaccttccagctgacctggagcctgatctgcaccgcccaggccatcttcctgatgaccaagtggcccaaggtgggctgggaggccatctacatgccctgctgcgaggccctgagctacggcctgctgagcgccggccacggctacctgaagctgcacggcgagtggcactacatcccctggatccgctacgccgcctggatggtgaccacccccgtgctgctgaaccaggtgcagggcgtgctggacgtgaaggtgggccacctgcgcctggcccccatccagagcctggccaacctgatcatgatcgtgatgggcgtgagcgcccacctgcaggagaacatcgcctggaagtgggtgttcttcctgatcggctgcgtgatggccctggtgatctactgggtggcctggctggtgttcagcacccagatcaagtactacgccgagaccaagaccgccgtgggcgaccgcgtggtgttccagctgaagctgatcgccgtggccttctacgtgggctggagcgccttccccctgatctggatgctgagcagcgagggcctgtgcgccatcgaggagcacatcgccgccgccgcccacatccccgccgacgtgctgagcaagaacatcttcggcctgctgatgtggcacacccgctggatcgtgctgggcggctactgggacgtgaacaagttcaaggagaccatcgcccagcagaagctggaggaggccaagggcgaggagaccccccaggagaagcgcaaggccctgatgggccaggccgaggcctgcttcaagatggccgacgcctgc

SEQ ID NO: 81 - DNA, 918 bp, Artificial Sequence
  synthetic humanized rhodopsin (7 TM) domain (RaACR_687)
atgggcgacatgagccacatcgagaccgtgtactacgtgaacggcaccgaggtggacccctggacccacagatgccagctgggcgtgcccggcggcagccagggcatccacaccttccagctgacctggagcctgatctgcaccgcccaggccatcttcctgatgaccaagtggcccaaggtgggctgggaggccatctacatgccctgctgcgaggccctgagctacggcctgctgagcgccggccacggctacctgaagctgcacggcgagtggcactacatcccctggatcagatacgccgcctggatggtgaccacccccgtgctgctgaaccaggtgcagggcgtgctggacgtgaaggtgggccacctgagactggcccccatccagagcctggccaacctgatcatgatcgtgatgggcgtgagcgcccacctgcaggagaacatcgcctggaagtgggtgttcttcctgatcggctgcgtgatggccctggtgatctactgggtggcctggctggtgttcagcacccagatcaagtactacgccgagaccaagaccgccgtgggcgacagagtggtgttccagctgaagctgatcgccgtggccttctacgtgggctggagcgccttccccctgatctggatgctgagcagcgagggcctgtgcgccatcgaggagcacatcgccgccgccgcccacatccccgccgacgtgctgagcaagaacatcttcggcctgctgatgtggcacaccagatggatcgtgctgggcggctactgggacgtgaacaagttcaaggagaccatcgcccagcagaagctggaggaggccaagggcgaggagaccccccaggagaagagaaaggccctgatgggccaggccgaggcctgcttcaagatggccgacgcctgc

SEQ ID NO: 82 - DNA, 936 bp, Artificial Sequence
  synthetic humanized rhodopsin (7 TM) domain (RlACR_497)
atggagtaccagagcctggacgacttccccctggagaccctgtacttccgcaacggcaccgaggtggacccctacgaccaccgctgcaagctgggcatccccggcggcagccagggcatccacaccttccagctggtgtgggccctgctgtgcgccgcccaggccctgttcctgttcagcaagtggcccaaggtgggctgggaggccatgtacatgccctgctgcgaggccctgagctacggcctgctgagcagcggctacggctacatgaagctgcacggcgagttccactacctgccctggatccgctacgccgcctggatcgtgaccacccccgtgctgctgaaccaggtgcagggcatgatcgacatcaagatcggcagcctgcgcctggcccccatccagagcctggccaacctgatcatgatcatcatgggcgtgagcgcccacctgcaggagaaccacgcctggaagtggctgttcttcttcgtgggctgcgtgatgtgcgccgtgatctactacatcgcctggctgcagttcgccaaggtgaacgagctgtacgccgccaccaagaccgagatcggcgacaaggtgtgcttccagctgaagctgatcatgttctgcttctacgtgggctggagcatgttcagcgtgatctggctgctgagcagcgagggcctgtgcctgatcgaggagcacatcagcgccgccgcccacatccccggcgacatcctgagcaagaacagcttcggcctgctgatgtggtacacccgctggatcaccctgggcggcgcctgggacatcaacaagttcctggagaagcaggccaaggagaaggccgaggccgtgctgacccccgccgagaagcgcaaggccatcatgaagcaggccgaggcctgcttcagcatggccgacgcctgcctgcagcaggccaac

SEQ ID NO: 83 - DNA, 936 bp, Artificial Sequence
  synthetic humanized rhodopsin (7 TM) domain (RlACR_497)
atggagtaccagagcctggacgacttccccctggagaccctgtacttcagaaacggcaccgaggtggacccctacgaccacagatgcaagctgggcatccccggcggcagccagggcatccacaccttccagctggtgtgggccctgctgtgcgccgcccaggccctgttcctgttcagcaagtggcccaaggtgggctgggaggccatgtacatgccctgctgcgaggccctgagctacggcctgctgagcagcggctacggctacatgaagctgcacggcgagttccactacctgccctggatcagatacgccgcctggatcgtgaccacccccgtgctgctgaaccaggtgcagggcatgatcgacatcaagatcggcagcctgagactggcccccatccagagcctggccaacctgatcatgatcatcatgggcgtgagcgcccacctgcaggagaaccacgcctggaagtggctgttcttcttcgtgggctgcgtgatgtgcgccgtgatctactacatcgcctggctgcagttcgccaaggtgaacgagctgtacgccgccaccaagaccgagatcggcgacaaggtgtgcttccagctgaagctgatcatgttctgcttctacgtgggctggagcatgttcagcgtgatctggctgctgagcagcgagggcctgtgcctgatcgaggagcacatcagcgccgccgcccacatccccggcgacatcctgagcaagaacagcttcggcctgctgatgtggtacaccagatggatcaccctgggcggcgcctgggacatcaacaagttcctggagaagcaggccaaggagaaggccgaggccgtgctgacccccgccgagaagagaaaggccatcatgaagcaggccgaggcctgcttcagcatggccgacgcctgcctgcagcaggccaac

SEQ ID NO: 84 - DNA, 921 bp, Artificial Sequence
  synthetic humanized rhodopsin (7 TM) domain (RsACR_665)
atggactacaccagcctggacgacttccccctggagaccctgtacttccgcaacggcaccgaggtggacccctacgaccaccgctgcaagctgggcatccccggcggcagccagggcatccacaccttccagctggtgtgggccctgctgtgcgccgcccaggccctgttcctgttcagcaagtggcccaaggtgggctgggaggccatgtacatgccctgctgcgaggccctgagctacggcctgctgagcagcggctacggctacatgaagctgcacggcgagttccactacctgccctggatccgctacgccgcctggctggtgaccacccccgtgctgctgaaccaggtgcagggcatgatcgacatcaagatcggcagcctgcgcctggcccccatccagagcctggccaacctgatcatgatcatcatgggcgtgagcgcccacctgcaggagaaccacgcctggaagtggctgttcttcttcgtgggctgcgtgatgtgcgccgtgatctactacatcgcctggctgcagttcgccaaggtgaacgagctgtacgccgccaccaagaccgagatcggcgacaaggtgtgcttccagctgaagctgatcatgttctgcttctacgtgggctggagcatgttcagcgtgatctggctgctgagcagcgagggcctgtgcctgatcgaggagcacatcagcgccgccgcccacatccccggcgacatcctgagcaagaacagcttcggcctgctgatgtggtacacccgctggatcaccctgggcggcgcctgggacatcaacaagttcctggagaagcaggccaaggagaaggccgaggccgtgctgacccccgccgagaagcgcaaggccatcatgaagcaggccgaggcctgcttcagcatggccgacgcctgc

SEQ ID NO: 85 - DNA, 921 bp, Artificial Sequence
  synthetic humanized rhodopsin (7 TM) domain (RsACR_665)
atggactacaccagcctggacgacttccccctggagaccctgtacttcagaaacggcaccgaggtggacccctacgaccacagatgcaagctgggcatccccggcggcagccagggcatccacaccttccagctggtgtgggccctgctgtgcgccgcccaggccctgttcctgttcagcaagtggcccaaggtgggctgggaggccatgtacatgccctgctgcgaggccctgagctacggcctgctgagcagcggctacggctacatgaagctgcacggcgagttccactacctgccctggatcagatacgccgcctggctggtgaccacccccgtgctgctgaaccaggtgcagggcatgatcgacatcaagatcggcagcctgagactggcccccatccagagcctggccaacctgatcatgatcatcatgggcgtgagcgcccacctgcaggagaaccacgcctggaagtggctgttcttcttcgtgggctgcgtgatgtgcgccgtgatctactacatcgcctggctgcagttcgccaaggtgaacgagctgtacgccgccaccaagaccgagatcggcgacaaggtgtgcttccagctgaagctgatcatgttctgcttctacgtgggctggagcatgttcagcgtgatctggctgctgagcagcgagggcctgtgcctgatcgaggagcacatcagcgccgccgcccacatccccggcgacatcctgagcaagaacagcttcggcctgctgatgtggtacaccagatggatcaccctgggcggcgcctgggacatcaacaagttcctggagaagcaggccaaggagaaggccgaggccgtgctgacccccgccgagaagagaaaggccatcatgaagcaggccgaggcctgcttcagcatggccgacgcctgc

SEQ ID NO: 86 - DNA, 855 bp, Artificial Sequence
  synthetic humanized rhodopsin (7 TM) domain (R1ACR_653)
atgccctacgacaactacggctggtgggagcgcgagaacgccgactgcatcagcaagatggacgccaagctggaccagatgcacctggtgcagctggccatgagcatgctgtgcttctgccagctggtgttcatgaccagccgcttccccaagcaggcctgggaggccatctacctgcccgccgccgagtgcgtgctgtacgccatggccgccagcggcaacggctacctgcgcctggccaacggccgcctgctgccctgggcccgcatggccagctggctggccacctgccccgtgatgctgaaccagatcaacaccatcagccccctgaagctgtacggcatcaacctgaacaacgtgcagatcgccctgagcgtggtgcgcgtggtgaacggcgtgagcgccagcagcgtggacgacgacagcctgaagtggggcttcttcttcatgggcgtgttctgcctggtgatcgagttcagcatcgcctacaacatcttcgccctgggcatggccaagttcaaggaggccggcaaggacagcgacatctgcgcccgcgcctgcgaccgcctgcaggtgctgcgcggcatcttcttcagcgcctgggccatgttccccatcatctggctgttcagcccccagggcacctgcctgctgggcgaggacgtgatccccctgctgtacctgttcgccgacctggtgtgcaagaactgcttcggcatcctgtggtacgacaccacctggcgcctgctggacggcaagggctggaaccccgagctgcagctgaccgacaacgaggtggaggagctggtgcacgccaaggaggaggtggaggaccaccaccacccccacgacgcc

SEQ ID NO: 87 - DNA, 855 bp, Artificial Sequence
  synthetic humanized rhodopsin (7 TM) domain (R1ACR_653)
atgccctacgacaactacggctggtgggagagagagaacgccgactgcatcagcaagatggacgccaagctggaccagatgcacctggtgcagctggccatgagcatgctgtgcttctgccagctggtgttcatgaccagcagattccccaagcaggcctgggaggccatctacctgcccgccgccgagtgcgtgctgtacgccatggccgccagcggcaacggctacctgagactggccaacggcagactgctgccctgggccagaatggccagctggctggccacctgccccgtgatgctgaaccagatcaacaccatcagccccctgaagctgtacggcatcaacctgaacaacgtgcagatcgccctgagcgtggtgagagtggtgaacggcgtgagcgccagcagcgtggacgacgacagcctgaagtggggcttcttcttcatgggcgtgttctgcctggtgatcgagttcagcatcgcctacaacatcttcgccctgggcatggccaagttcaaggaggccggcaaggacagcgacatctgcgccagagcctgcgacagactgcaggtgctgagaggcatcttcttcagcgcctgggccatgttccccatcatctggctgttcagcccccagggcacctgcctgctgggcgaggacgtgatccccctgctgtacctgttcgccgacctggtgtgcaagaactgcttcggcatcctgtggtacgacaccacctggagactgctggacggcaagggctggaaccccgagctgcagctgaccgacaacgaggtggaggagctggtgcacgccaaggaggaggtggaggaccaccaccacccccacgacgcc

SEQ ID NO: 88 - DNA, 891 bp, Artificial Sequence
  synthetic rhodopsin R1ACR_6367
atgagcgccatcaccgaccccgccctgggcctgtacggcacctgggaggccgccaacaaggagtgcgtggacaagggcgtggagttcagcagcaacacccgcatgatcgtgctgctgttcgccagcgtgtgcttctgccagaccgtgtacatgaccagccgcgccccccgcgtgaactgggagggctgctacctgcccctgtgcgagaccttcctgtacggcctggccgccagcggcaacggcatgatccgcctgagcaacggccgcatggtgcccatggcccgcttctgcagctggctggtgacctgccccatcatgctgatgagcgtgctggccatccacgacgtgcgctggatgggcgtgcccgtgaagaacatggtgatcgccgccaccctgatccgcaccgtgctgggcatcggcgccagcagcaccatcaccgagaccctgcgctgggtgcagtactgcatcgcctgcgccgtgttcgtgttcgagctgaccatggtgaaccagctgttcatcgccggcctgcagaagttcggcgtggtgaagaccaagctgaacgacatggtgtacatgcgcatcatgatcctgcgcgccctgttcttcttcagctggaccgccttccccgtgatctggatcctgagcagcaccggcttctgcgtgatcggcgaggacggcagcgtgctggcctacctggtggccgacttcctgtgcaagaacgtgtacggcatcatcaacaccaacaccctgttccaggtgctggacggcgagtggaagcccgagacccgcgacaccgacggcaagatcgccaccaaccgccacgcccacatcggcgagacctacctggagatgctgctggcccgcatccgccgccaccgcgagggcgagctg

SEQ ID NO: 89 - DNA, 891 bp, Artificial Sequence
  synthetic humanized rhodopsin (7 TM) domain (R1ACR_6367)
atgagcgccatcaccgaccccgccctgggcctgtacggcacctgggaggccgccaacaaggagtgcgtggacaagggcgtggagttcagcagcaacaccagaatgatcgtgctgctgttcgccagcgtgtgcttctgccagaccgtgtacatgaccagcagagcccccagagtgaactgggagggctgctacctgcccctgtgcgagaccttcctgtacggcctggccgccagcggcaacggcatgatcagactgagcaacggcagaatggtgcccatggccagattctgcagctggctggtgacctgccccatcatgctgatgagcgtgctggccatccacgacgtgagatggatgggcgtgcccgtgaagaacatggtgatcgccgccaccctgatcagaaccgtgctgggcatcggcgccagcagcaccatcaccgagaccctgagatgggtgcagtactgcatcgcctgcgccgtgttcgtgttcgagctgaccatggtgaaccagctgttcatcgccggcctgcagaagttcggcgtggtgaagaccaagctgaacgacatggtgtacatgagaatcatgatcctgagagccctgttcttcttcagctggaccgccttccccgtgatctggatcctgagcagcaccggcttctgcgtgatcggcgaggacggcagcgtgctggcctacctggtggccgacttcctgtgcaagaacgtgtacggcatcatcaacaccaacaccctgttccaggtgctggacggcgagtggaagcccgagaccagagacaccgacggcaagatcgccaccaacagacacgcccacatcggcgagacctacctggagatgctgctggccagaatcagaagacacagagagggcgagctg

SEQ ID NO: 90 - DNA, 915 bp, Artificial Sequence
  synthetic humanized rhodopsin (7 TM) domain (R1ACR_591)
atggtgtggagccacgtggacgagcccgccctgggcccctacggcagctgggaggccgccaaccccgactgcgtggccctgggcctgaccaccagcaagaacaccaagatgatcctgctgctgttcgccagcatctgcgcctgccaggtgctgttcatggccagccgcgccccccgcatcacctgggaggccgtgtacctgcccctgatcgagaccatcctgtacgccatggccagcaccggcaacggccagctgcgcctgggcgacggccgcgtgctgcccatcgcccgctactgcagctggctgatcacctgccccatcatgctgttccaggtggtgggcctgtacgacctgaagctgtggggcatcagctgcaagcacatggtgatggccgccgccctgatccgcaccgtgttcggcatcggcgccagcgtgaccctggacgaccagatgcgctgggtgcagttcttcctgagctacgccttcttcttcttcgagctgtactgcgcctacatgatcttcggcgccgccctgaagcagttcggcgaggtgcgcaccaccctgaacagcatcgtgtacagccgcatcctgctgctgcgcttcatcttcttcctgagctggaacagcttcggcatcatctggctgctgagcagcaccggcctgtgcatgatcagcgaggacgtgagcgtgctggcctacctggccgccgacatgctgtgcaagaacacctacggcatcatcaacgccaacaccctgttccgcgtgctggacggcaagtggacccccgagaccctggaggaggccgccagcggcgtgaagaaggccaaccccaaccacgtgggcgagacctacctggagcgcatgatggccggcatccgcggcagcgccaagcccgaggaggaccccgtggccgcc

SEQ ID NO: 91 - DNA, 915 bp, Artificial Sequence
  synthetic humanized rhodopsin (7 TM) domain (R1ACR_591)
atggtgtggagccacgtggacgagcccgccctgggcccctacggcagctgggaggccgccaaccccgactgcgtggccctgggcctgaccaccagcaagaacaccaagatgatcctgctgctgttcgccagcatctgcgcctgccaggtgctgttcatggccagcagagcccccagaatcacctgggaggccgtgtacctgcccctgatcgagaccatcctgtacgccatggccagcaccggcaacggccagctgagactgggcgacggcagagtgctgcccatcgccagatactgcagctggctgatcacctgccccatcatgctgttccaggtggtgggcctgtacgacctgaagctgtggggcatcagctgcaagcacatggtgatggccgccgccctgatcagaaccgtgttcggcatcggcgccagcgtgaccctggacgaccagatgagatgggtgcagttcttcctgagctacgccttcttcttcttcgagctgtactgcgcctacatgatcttcggcgccgccctgaagcagttcggcgaggtgagaaccaccctgaacagcatcgtgtacagcagaatcctgctgctgagattcatcttcttcctgagctggaacagcttcggcatcatctggctgctgagcagcaccggcctgtgcatgatcagcgaggacgtgagcgtgctggcctacctggccgccgacatgctgtgcaagaacacctacggcatcatcaacgccaacaccctgttcagagtgctggacggcaagtggacccccgagaccctggaggaggccgccagcggcgtgaagaaggccaaccccaaccacgtgggcgagacctacctggagagaatgatggccggcatcagaggcagcgccaagcccgaggaggaccccgtggccgcc

SEQ ID NO: 92 - DNA, 720 bp, Artificial Sequence
  synthetic humanized rhodopsin (7 TM) domain (R1ACR_053)
atgggcctggacaccagcagccagaccaacatgatccagctgctgttcgccagcatctgcttctgccaggtgctgttcatggccagccgcgccccccgcgtgacctgggaggccgtgtacctgcccctggtggagaccgtgctgtacggcatggccgccagcggcaacggcaacctgcgcatcagcgacggccgcatggtgcccatggcccgcttctgcagctggctgatcacctgccccatcatgctgctgcaggtggtggtgatgcacgacgtggtgtggttcaacgtgagcacccgctacatggtgctggccgccagcctgatccgcaccgtgttcggcatcggcgccagcgtgaccaccaacgacaccatgcgctgggtgcagttcctgctgagctgcatctgcttcgccttcgagatggtggtggcccaccagacctacaagggcgccctgaagaagttcgccgccgtgaagaccaccctgaacggcgtggtgtacgcccgcatcatgttcctgcgcttcctgttcttcttcagctggaacgccttccccatcatctgggccctgagcagcaccggcctgtgcctgatcagcgaggacgccagcgtgctgagctacctggtggccgacctgatctgcaagaacatctacggcatcaccaacgccaacaccgtgttccgcgtgctggacggcgcctgggtgcccgagaccgaggag

SEQ ID NO: 93 - DNA, 720 bp, Artificial Sequence
  synthetic humanized rhodopsin (7 TM) domain (R1ACR_053)
atgggcctggacaccagcagccagaccaacatgatccagctgctgttcgccagcatctgcttctgccaggtgctgttcatggccagcagagcccccagagtgacctgggaggccgtgtacctgcccctggtggagaccgtgctgtacggcatggccgccagcggcaacggcaacctgagaatcagcgacggcagaatggtgcccatggccagattctgcagctggctgatcacctgccccatcatgctgctgcaggtggtggtgatgcacgacgtggtgtggttcaacgtgagcaccagatacatggtgctggccgccagcctgatcagaaccgtgttcggcatcggcgccagcgtgaccaccaacgacaccatgagatgggtgcagttcctgctgagctgcatctgcttcgccttcgagatggtggtggcccaccagacctacaagggcgccctgaagaagttcgccgccgtgaagaccaccctgaacggcgtggtgtacgccagaatcatgttcctgagattcctgttcttcttcagctggaacgccttccccatcatctgggccctgagcagcaccggcctgtgcctgatcagcgaggacgccagcgtgctgagctacctggtggccgacctgatctgcaagaacatctacggcatcaccaacgccaacaccgtgttcagagtgctggacggcgcctgggtgcccgagaccgaggag

SEQ ID NO: 94 - DNA, 906 bp, Artificial Sequence
  synthetic humanized rhodopsin (7 TM) domain (C1ACR_561)
atggagatcaacaccaccagcccctacggctggtgggccgccgccaaccccgagtgcgtggccaaggccgccgtgaccgtggacatccagatcttccagctgtgctggtgcatcatctgcgcctgccaggccttcttcttcgccatccgctaccccaaggtgacctgggagttcgtgctggtgcccctgaccgaggccgtgatctacggcctgggctacagcggcatcggctacgtggtgctgcagagcggccgcaccgtggcctggatgcgcatctgccactggctgatcaccgtgcccgtgctgctgctgcagatcagcgacgtggcccacgtgcgcttccacggcgtgaacctgagcagcctgcaggtgcccatcagcgtgctgatgatcgtgttcggcttctgcgccgaggtggccgacgaggagggcgtgaagtggatgttcttcgtgatcggcctggtgctgatgttccaggtgctgttcgtgggctaccacagcctgagccgcggcatcacccgcttcagcgaggccggcaccgaggagggcgccatggtggccaaccgcctgcgcgccatgctggtgttcttcctgctgagctggaccctgcaccccctgttcttcgtgctgagcatcgagggcgcctgcgtgatgaccgagcccgtgaccgtggtgctgtacatcgtgctggacctgttcgccaagaacatcttccagatcgtgctgtgggacagcctgtgggtgcgcctgaccggcaagtggaaggtggaggagctggtggtgcacgacgccagcgccgagctgggctacgacgaggactacaagcacgacgagatcaacctgagcatcggcaacgtgcccgtgagcaccgagagccccgtgccccgcggcgccggcgagggcgcc

SEQ ID NO: 95 - DNA, 906 bp, Artificial Sequence; identical to SEQ ID NO: 94
  synthetic humanized rhodopsin (7 TM) domain (C1ACR_561)

SEQ ID NO: 96 - DNA, 861 bp, Artificial Sequence
  synthetic humanized rhodopsin (7 TM) domain (R2ACR_041)
atgggcctgaccgtgaccagcaacaccaaggtgttccagctgctgttcgcctgcgtgtgcgcctgccaggtgctgttcatggccagccgcgccccccgcgtgacctgggaggccgtgtacctgcccctggtggagaccatcctgtacgccatggccgccagcggcaacggcagcatgcgcctggccgacggccgcgtgatccccatcggccgcttctgcagctggctgatcacctgccccatcatgctgttccaggtggtgggcatccacgagctgaagtggagcggcgtgagctgcaagaacatggtgatggccgccagcctgatccgcaccgtgtacggcatcggcgccagcgtggtggccaacgacatcatgaagtgggtgctgtacgtgctgggctgcgtgtgcctggccttcgagttcctggtggtgcacagcatcttcagcgccggcctgaagaagttcgccgccttccgcagcaccctgaacgacggcgtgtacaaccgcctgctgttcatgcgcgccctgttcttcaccgcctggaccagcttccccgtgatctgggtgctggccagcaccggcatctgcctgatcgacgagaacgtgaccgtgctggcctacctgatcgccgacatgctgtgcaagaacatgtacggcatcatcaacgccaacaccctgttccgcgtgctggacggcagctggacccccgagaccctggagaccagcaagaacccccccaagcacgaggtgcacgagacctacatcgacatggccatgcaccacatgcgccccagcaagagcaagacccgcgaggccgccatcgagctggaggagcacaccgccgaccccttcgccgagggc

SEQ ID NO: 97 - DNA, 861 bp, Artificial Sequence
  synthetic humanized rhodopsin (7 TM) domain (R2ACR_041)
atgggcctgaccgtgaccagcaacaccaaggtgttccagctgctgttcgcctgcgtgtgcgcctgccaggtgctgttcatggccagcagagcccccagagtgacctgggaggccgtgtacctgcccctggtggagaccatcctgtacgccatggccgccagcggcaacggcagcatgagactggccgacggcagagtgatccccatcggcagattctgcagctggctgatcacctgccccatcatgctgttccaggtggtgggcatccacgagctgaagtggagcggcgtgagctgcaagaacatggtgatggccgccagcctgatcagaaccgtgtacggcatcggcgccagcgtggtggccaacgacatcatgaagtgggtgctgtacgtgctgggctgcgtgtgcctggccttcgagttcctggtggtgcacagcatcttcagcgccggcctgaagaagttcgccgccttcagaagcaccctgaacgacggcgtgtacaacagactgctgttcatgagagccctgttcttcaccgcctggaccagcttccccgtgatctgggtgctggccagcaccggcatctgcctgatcgacgagaacgtgaccgtgctggcctacctgatcgccgacatgctgtgcaagaacatgtacggcatcatcaacgccaacaccctgttcagagtgctggacggcagctggacccccgagaccctggagaccagcaagaacccccccaagcacgaggtgcacgagacctacatcgacatggccatgcaccacatgagacccagcaagagcaagaccagagaggccgccatcgagctggaggagcacaccgccgaccccttcgccgagggc

SEQ ID NO: 98 - protein, 13 aa, Artificial Sequence
  Synthetic peptide
GRKKRRQRRRPPQ

SEQ ID NO: 99 - protein, 16 aa, Artificial Sequence
  Synthetic peptide
RQIKIWFQNRRMKWKK

SEQ ID NO: 100 - protein, 26 aa, Artificial Sequence
  Synthetic peptide
GIGAVLKVLTTGLPALISWIKRKRQQ

SEQ ID NO: 101 - protein, 10 aa, Artificial Sequence
  Synthetic peptide
TKIESLKEHG

SEQ ID NO: 102 - protein, 10 aa, Artificial Sequence
  Synthetic peptide
TQIENLKEKG

SEQ ID NO: 103 - protein, 26 aa, Artificial Sequence
  Synthetic peptide
AALEALAEALEALAEALEALAEAAAA

SEQ ID NO: 104 - protein, 25 aa, Artificial Sequence
  Synthetic peptide
GLFEAIEGFIENGWEGMIEGWYGCG

SEQ ID NO: 105 - protein, 301 aa, Artificial Sequence
  synthetic rhodopsin C1ACR_023
MTVLEVNEAVIIDGVMYGEYIFSHPQCIPAELVPVVKVFQICWAVVSMCQSAFMMTRMPKVPWEAIYLPMVEAIVYVMAYTGNGYLKMGSGRVLPWARMASWLVTCPVMHGQISGMTLIKYQSIPINPMVIAASIIRVVMGVTATIVVTEDLKWAFFGCGLVCLLFEYASVYTIFAVTIGDFADAKTDLGNQVVARLKSIRSIFFVAWTAFPILWLLSSTGTCVIDENISAVAYLISDAFCKNFNGILLWSTTWGLLNGKWDRDFVRAPPAAESEKDVQDAPRPKSDVEVKMFGKVIGSVR

SEQ ID NO: 106 - protein, 294 aa, Artificial Sequence; identical to SEQ ID NO: 9
  synthetic rhodopsin R1ACR_367

SEQ ID NO: 107 - protein, 316 aa, Artificial Sequence
  synthetic rhodopsin GcACR_457
MSACPPRTPRYGNMSAADKRAEYLQKVIDLSPEIYGSYARDNAECLAMSVVTMPFVKITQLCMAVMSACQVLFMISRAPRVPWEAIYLPATEMISYSLAFFGQGYVRLANGKVLPWCRMAAWLCTCPIMLGLVSNMALIKYKSQPLNPMMIAASIIRTVFGISATMAETNDVIWTHFFFAFVCFIFEMSCAFAIFALTIDDFQQIGSPLAMKVVKRLYLLRMVFFGTWSCFPILWIISSTYMCVIDENASALLYLVADISCKNCYGLLLWSTTWGLLNGKWDREYARNRDEAGMLMETDEKQGVIEPPKENFDVKL

SEQ ID NO: 108 - protein, 351 aa, Artificial Sequence
  synthetic rhodopsin GcACR_203
MAARLAKVVHLARLVVLFAVMLDAQDTGSQAVGFQPEERMVVRGKAAYIDGIGPATPYAGMYGEWEIANPDCVNKGLHATSRERTIITLWAVVCLCQALFMMTRAPKVTWEAVYLPLVECILYCCTGAGYGVLRMGDGRMLPLSRMCSWLITCPIMLSQAFGIHDMKLYGIQLRMPICAASLIRTVCGLLASTTTDTKIAKWLFFFLGFSFYTMEITTVWFIFQQGIAKFTPTTDVPENATALRRLYFMRFIFFTSWTAFPVFWVLSSTGLCVISEQTSALGYLCADALCKNLYGVIQTNTVWGLLGGTWTPKKHPNNHDEEAPANSHNHKEEDTFAAPHGRFNSAKEIED

SEQ ID NO: 109 - protein, 280 aa, Artificial Sequence
  synthetic rhodopsin C1ACR_197
MQASQISSSYFGQWSATNIDCVAKADISSGVQTFLLCFCIISACQALYFSYHCLPQAGARRRSTWEFIAVPAVEALCYGLASSSSQVGYFRLFDGRTVVWLRTVLWILTVPVLLLQIGKMSYVVIQGVEVNSLIIFVGLLMVVFGFSASLSTVMGIKWMFFFFGLVCMAVVFLGVYKIMVDAGSYYISLNSQDGLQIASRIRILMIIFFFSWTATPLFWVLSIEGACVVSESITVVCFAVIDSLAKNIFGIILWDTVWNSSLEGRWTSNLQIMATEMSML

SEQ ID NO: 110 - protein, 297 aa, Artificial Sequence
  synthetic construct R1ACR_741
MSYVEDPAFGFYGSWEAANQDCIERGLSTSNNTRMIQLLFASICACQVLFMASRAPRVTWEAVYLPVVETVLYGMASTGNGNMRLADGRIVPIARFCSWLITCPIMLFQVVGIHELKWRTISCKNMVMAASLIRTVFGIGASVTITPTMRWVQYFLSLSFFLFELIVVYQIFSAGLKKFGAVKTSLNNIVYSRIMFLRFLFFLSWSSFPIIWLLSSTGFCLVNEDVSVLAYLVADMICKNLYGITNANTLFRVLNGKWTPETEEEAMLPKDHEHAHVGETYFEQMLSGLRGSRRRDH

SEQ ID NO: 111 - protein, 300 aa, Artificial Sequence
  synthetic construct R1ACR_447
MVWSHVDEPALGPYGSWEAANPDCVALGLTTSKNTKMILLLFASICACQVLFMASRAPRITWEAVYLPLIETILYAMASTGNGQLRLGDGRVLPIARYCSWLITCPIMLFQVVGLYDLKLWGISCKHMVMAAALIRTVFGIGASVTLDDQMRWVQFFLSYAFFFFELYCAYMIFGAALKQFGEVRTTLNSIVYSRIMLLRFIFFLSWNSFGIIWLLSSTGLCMISEDVSVLAYLAADMLCKNTYGIINANTLFRVLDGKWTPETLEEAASGVKKANPNHVGETYLERMMAGIRGSAKPEE

SEQ ID NO: 112 - protein, 300 aa, Artificial Sequence
  synthetic construct R1ACR_877
MSATENPAFGLYGTWENDHPQCVEMGLDTSSQTNMIHLLFASICFCQVLFMASRAPRVTWEAVYLPLVETVLYGMAASGNGNLRISNGRMVPMARFCSWLITCPIMLLQVVVMHDVVWFNVSTRYMVLAASLIRTVFGIGASVTTNDTMRWVQFLLSCICFAFEMVVAHQTYKGALKKFAAVKTTLNGVVYARIMFLRFLFFFSWNAFPIIWALSSTGLCLISEDASVLSYLVADLICKNIYGITNANTVFRVLDGAWVPETEEHAQAKDHLTYLEMMMNHNLGIGRHHDEELQMEVGHD

SEQ ID NO: 113 - protein, 275 aa, Artificial Sequence
  synthetic construct R1ACR_995
MGIASGAPVKIIQLCWAVVSLCQVIFMLTRAPRVAWEAIYLPLCETITYFLAFTGNGYLRMADGQIFPWARMASWLVTCPVMLGQVSAMALIKYKSLPLNNMMVAASLIRVVMGISATITPNEQLKWMFFFFGVSMLTFEYFCVFTIFSITIADFQEIDTPRSRLVVGRLKILRIVFHSAWTAFPILWVVSSTGLCVLSENISSVCYMLADAACKNGYGLILWNTTWGHLNGKWDREFAKNADKDGEMKAIEEGTAGPAADAKKDEDIKLFGKPV

SEQ ID NO: 114 - protein, 297 aa, Artificial Sequence
  synthetic construct R1ACR_477
MALYTSQFGIAYGSWEAENDDCLSMGIASGAPVKIIQLCWAVVSLCQVIFMLTRAPRVAWEAIYLPLCETITYFLAFSGNGYLRMADGQIFPWARMASWLVTCPVMLGQVSAMALIKYKSLPLNNMMVAASLIRVVMGISATITPNEQLKWMFFFFGVSMLTFEYFCVFTIFSITIADFQEIDTPRSRFVVGRLKILRIVFHSAWTAFPILWVVSSTGLCVLSENISSVCYMLADAACKNGYGLVLWNTTWGHLNGKWDREFAKNADKDGEMKAIEEGAAGPAADAKKDEDIKLFGK

SEQ ID NO: 115 - protein, 301 aa, Artificial Sequence
  synthetic construct R1ACR_433
MAELVDEYGLWALENPECMEMSYTSPNTKTAQLCFCMVCLCQIIWIMGQWPKVGWESIYLPACEMTLYSLAASGNGFIQLADGRIIPYARMAAWICTCPALLMQINGLAKCKLGHFNLNTFQVVADLIMNVMGLNATLTNHTYLKWIYFGIAMVLFGCIMGITHDIFSQTARKWKSVDTPLAQKVAWRVTFLKYLFSLSWSIYPLLWILSPQATCSLREDVISCLHFVSDFFAKNIFGLVMWWTLWRDLDGDWSFEHHYPPDQEQARSSASATHSLPGKSIGLAESINFVKDTQDCHDHDG

SEQ ID NO: 116 - protein, 311 aa, Artificial Sequence
  synthetic construct R1ACR_973
MAALHQALEELGQGASRHLLYRAPEEMVYGKWVEANPRCIELHDHSDYVYVFQLCFAVVCACQVIFMFTRAPNVGWEAIYLPLAEVVTYSIAANGEGVLRMADGRYFNFAKLAGWAVCCPIMLIQIGGMAQIKYRTIPLNNVVLAASLNRIIFGMASAITASDPARWGFYFCAWICYLTEVGITLTIMAVAISDFSKIKTELGQWVVGRIQTMRIIFLVAWTSFPVVWVLGYTGFCVIHEDYIALLYLFADLLSKNTWGVMMWHTTWVKLNGKWDREFAAAGGHEALKKALEQDVEIGAGEKNQNQLTARQ

SEQ ID NO: 117 - protein, 291 aa, Artificial Sequence
  synthetic construct R1ACR_243
MIITLWAVVCMCQALFMMTRAPKVTWEAVYLPLVECILYCCTGAGYGQLRMGDGRMLPLSRMCSWLITCPIMLSQAFGIHDLKLYGIQMRMPILAASLIRSVCGLLASTTTDTKVVKWFFFFLGFCFFSMEIGSVWFIFQHGIAKFAPTTDVPENATALNRLYIMRVIFFSSWTAFPLFWVLSSTGLCVISEQTSALGYLCADAFCKNLYGVIQTNTVWGVLGGTWTPKKPPSKDDEEAPTNSHNDKEMDAFTAPHGRFNSAKEIQDTYNSDTYFIRAAKAVSVRMSRKKG

SEQ ID NO: 118 - protein, 340 aa, Artificial Sequence
  synthetic construct R1ACR_439
MAETGVIRSQEDYYRLVENRETIGTYWDSSLVYGEWARNNGNCLRQSEVDVQFVKILQLCMSVVSSCQVLFMVSRAPLVPWEAIYLPMTESITYGLAFTGNGYIRLATGKILPWARMASWLCTCPIMLGMVSNMALVKYKSQPLNPLMVASSIIRVVFGISATMSDDSQPAKWVFFFIAVIFFLFELTCVYAIFGMTIADFAQIQSPLGDAVVYRLNILRGIFFASWTCFPILWLVSSTSACLLDENISAVLYLMADALCKNSYGIVLWSTTWGLLNGKWDREYPRNRDIDGLMMDIDPAKEKQLEEMPNQNFDIKIMGQTIASVKRSCRRPRADMESQD